(19) United States
(12) Patent Application Publication
(10) Pub. No.: US 2015/0175651 A1
(43) Pub. Date: Jun. 25, 2015

(54) NEUROACTIVE STEROIDS, COMPOSITIONS, AND USES THEREOF

(71) Applicant: SAGE THERAPEUTICS, INC., Cambridge, MA (US)

(72) Inventors: Francesco Gerald Salituro, Marlborough, MA (US); Albert Jean Robichaud, Cambridge, MA (US)

(73) Assignee: SAGE THERAPEUTICS, INC., Cambridge, MA (US)

(21) Appl. No.: 14/408,045

(22) PCT Filed: Jun. 14, 2013
(86) PCT No.: PCT/US13/45933

§ 371 Date: Dec. 15, 2014

Related U.S. Application Data
(60) Provisional application No. 61/660,519, filed on Jun. 15, 2012.

(51) Int. Cl. C07J 41/00; C07J 1/00; C07J 7/00

(52) U.S. Cl. CPC C07J 41/0094; C07J 7/002; C07J 1/0029

(57) ABSTRACT

Described herein are neuroactive steroids of the Formula (1) or a pharmaceutically acceptable salt, solvate, stereoisomer, tautomer, and/or isotopic variant thereof. Such compounds are envisioned, in certain embodiments, to behave as soft drugs and, in certain embodiments, as GABA modulators. The present invention also provides pharmaceutical compositions comprising a compound of the present invention and methods of use and treatment, e.g., such for inducing sedation and/or anesthesia.

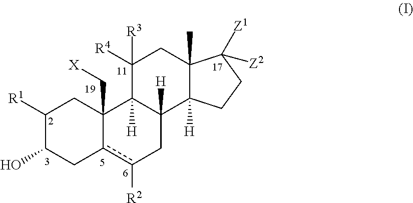

### CROSS-REFERENCE AND CLAIM OF PRIORITY

This application claims priority to U.S. provisional patent application Ser. No. 61/660,519 filed Jun. 15, 2012, the contents of which are incorporated herein by reference.

### BACKGROUND OF THE INVENTION

Brain excitability is defined as the level of arousal of an animal, a continuum that ranges from coma to convulsions, and is regulated by various neurotransmitters. In general, neurotransmitters are responsible for regulating the conductance of ions across neuronal membranes. At rest, the neuronal membrane possesses a potential (or membrane voltage) of approximately −70 mV, the cell interior being negative with respect to the cell exterior. The potential (voltage) is the result of ion (K⁺, Na⁺, Cl⁻, organic anions) balance across the neuronal semipermeable membrane. Neurotransmitters are stored in presynaptic vesicles and are released under the influence of neuronal action potentials. When released into the synaptic cleft, an excitatory chemical transmitter such as acetylcholine will cause membrane depolarization, e.g., a change of potential from −70 mV to −50 mV. This effect is mediated by postsynaptic nicotinic receptors which are stimulated by acetylcholine to increase membrane permeability to Na⁺ ions. The reduced membrane potential stimulates neuronal excitability in the form of a postsynaptic action potential.

In the case of the GABA receptor complex (GRC), the effect on brain excitability is mediated by GABA, a neurotransmitter. GABA has a profound influence on overall brain excitability because up to 40% of the neurons in the brain utilize GABA as a neurotransmitter. GABA regulates the excitability of individual neurons by regulating the conductance of chloride ions across the neuronal membrane. GABA interacts with its recognition site on the GRC to facilitate the flow of chloride ions down an electrochemical gradient of the GRC into the cell. An intracellular increase in the levels of this anion causes hyperpolarization of the transmembrane potential, rendering the neuron less susceptible to excitatory inputs, i.e., reduced neuron excitability. In other words, the higher the chloride ion concentration in the neuron, the lower the brain excitability and level of arousal.

It is well-documented that the GRC is responsible for the mediation of anxiety, seizure activity, and sedation. Thus, GABA and drugs that act like GABA or facilitate the effects of GABA (e.g., the therapeutically useful barbiturates and benzodiazepines (BZs), such as Valium®) produce their therapeutically useful effects by interacting with specific regulatory sites on the GRC. Accumulated evidence has now indicated that in addition to the benzodiazepine and barbiturate binding site, the GRC contains a distinct site for neuroactive steroids. See, e.g., Lan, N. C. et al., Neurochem. Res. (1991) 16:347-356.

### SUMMARY OF THE INVENTION

Provided herein are neuroactive steroids comprising at least one ester or carbonate group at one or more positions 2, 6, 11, and/or 19 on the steroid scaffold, and designed, for example, to act as GABA modulators. In certain embodiments, such compounds are envisioned to be useful as therapeutic agents for the inducement of anesthesia and/or sedation in a subject. In certain embodiments, such compounds are further envisioned to behave as soft drugs.

In one aspect, provided is a compound of Formula (I):

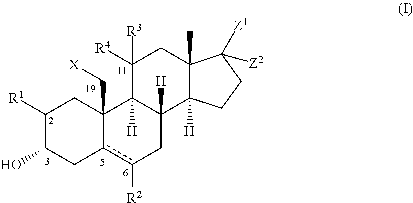

or a pharmaceutically acceptable salt, solvate, stereoisomer, tautomer, and/or isotopic variant thereof; wherein [symbol] , X, Z¹, Z², R¹, R², R³, and R⁴ are as defined herein;

provided at least one of R¹, R², R³, and X is an ester or carbonate group of the formula —OC(═O)R^{E1}; wherein R^{E1} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or —OR^{E2}, wherein R^{E2} is as defined herein.

Compounds of the present invention may, in certain embodiments, behave as “soft drugs.” A soft drug, as used herein, refers to a pharmacologically active compound which, after exerting a therapeutic effect, undergoes metabolism to a less active or inactive metabolite. See, e.g., Boder et al., Med. Res. Rev. (2000) 20:58-101. Soft drugs are considered the opposite of a pro-drug, since pro-drugs are hydrolyzed in vivo to more active compounds. It is contemplated herein that certain compounds of Formula (I), designed to comprise an alpha C₃—OH group, desirable for potent GABA modulation, and further designed to comprise at least one ester or carbonate group at certain positions known to play a lesser pharmacological role, e.g., at positions 2, 6, 11, and/or 19, are expected, upon administration, to exert a therapeutic effect in vivo prior to being metabolized to a less active or inactive alcohol. The inventors contemplate that appropriately designed compounds of the present invention should behave as potent anesthetics, that, when metabolized in vivo by non-specific esterases, for example, peripheral esterases, will become less active or inactive and thus allow for a faster recovery from sedation.

In certain embodiments, the compound of Formula (I) is selected from any one of the formulae:

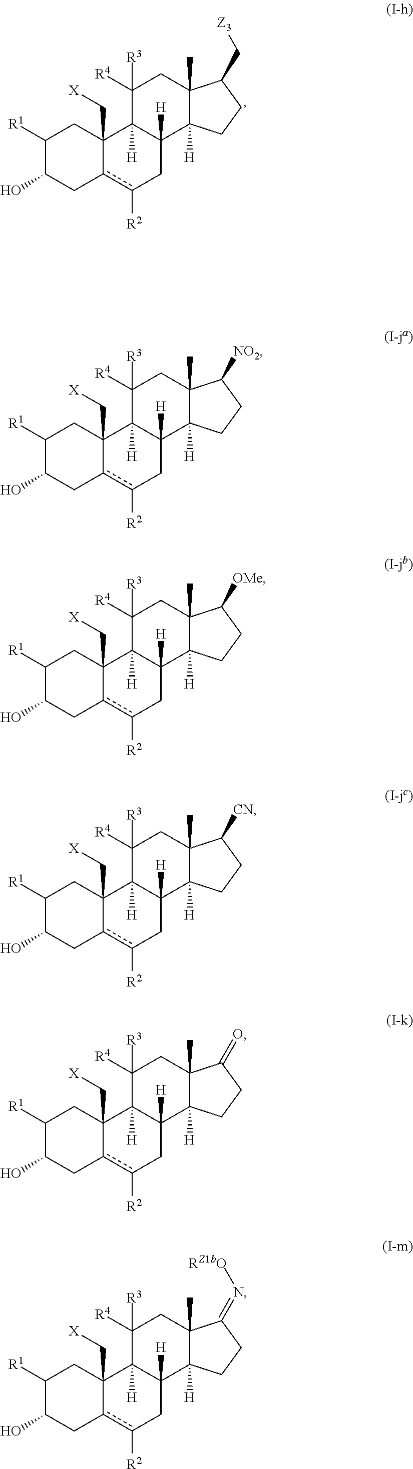

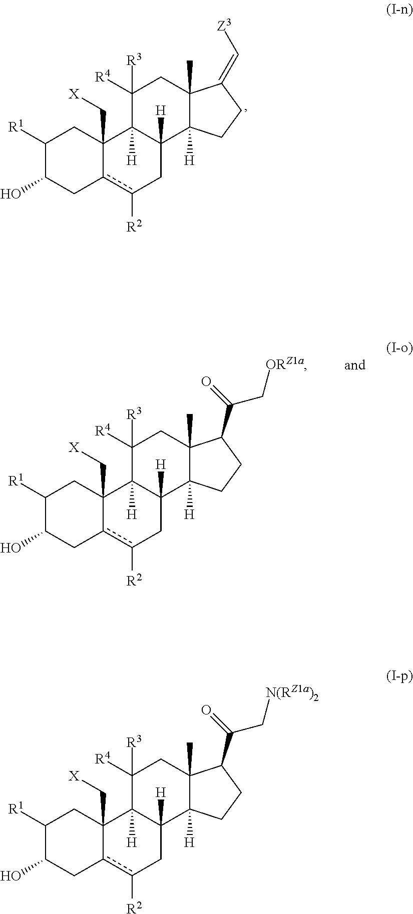

or a pharmaceutically acceptable salt, solvate, stereoisomer, tautomer, and/or isotopic variant thereof, wherein [symbol] , Z³, R^{Z1a}, R^{Z1b}, X, R¹, R², R³, and R⁴ are as defined herein.

Compounds of the present invention comprise at least one group of the formula —OC(═O)R^{E1} present on the scaffold at positions 2, 6, 11, and/or 19. In certain embodiments, only one of R¹, R², R³, and X is a group of the formula —OC(═O)R^{E1}. In certain embodiments, at least two of R¹, R², R³, and X is, independently, a group of the formula —OC(═O)R^{E1}. In certain embodiments, at least three of R¹, R², R³, and X is, independently, a group of the formula —OC(═O)R^{E1}. In certain embodiments, the compound of Formula (I) is selected from any one of the Formula:

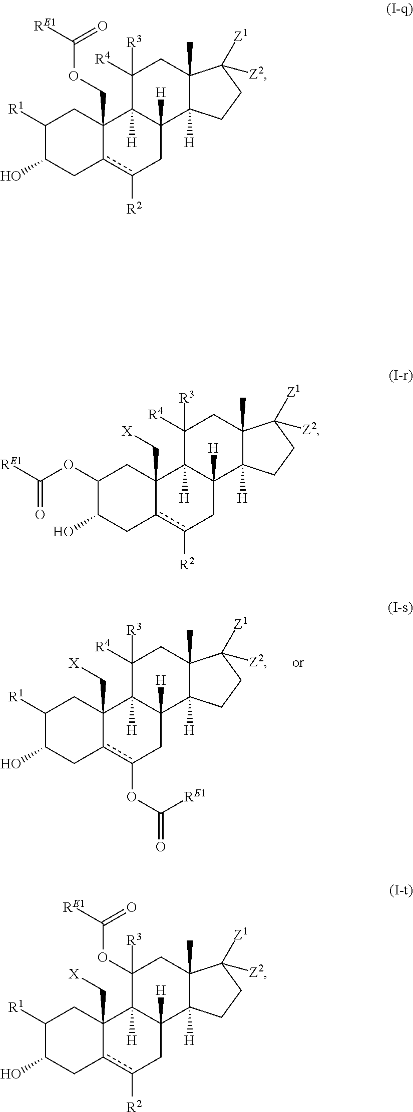

or a pharmaceutically acceptable salt, solvate, stereoisomer, tautomer, and/or isotopic variant thereof, wherein [symbol] , R^{E1}, Z¹, Z², X, R¹, R², R³, and R⁴ are as defined herein.

In another aspect, provided are pharmaceutical compositions comprising a compound of the present invention and a pharmaceutically acceptable excipient.

In yet another aspect, provided are methods of inducing sedation and/or anesthesia in a subject comprising administering to the subject an effective amount of a compound of the present invention, or a pharmaceutical composition thereof. In certain embodiments, the compound is administered by intravenous administration. In certain embodiments, the compound is metabolized in vivo to a less active or inactive compound.

Other objects and advantages will become apparent to those skilled in the art from a consideration of the ensuing Detailed Description, Examples, Figures and Claims.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 80% by weight R-compound and at most about 20% by weight S-compound, at least about 90% by weight R-compound and at most about 10% by weight S-compound, at least about 95% by weight R-compound and at most about 5% by weight S-compound, at least about 99% by weight R-compound and at most about 1% by weight S-compound, at least about 99.9% by weight R-compound or at most about 0.1% by weight S-compound. In certain embodiments, the weights are based upon total weight of compound.

As used herein and unless otherwise indicated, the term “enantiomerically pure S-compound” or “S-compound” refers to at least about 80% by weight S-compound and at most about 20% by weight R-compound, at least about 90% by weight S-compound and at most about 10% by weight R-compound, at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group substituted with one or more of groups selected from halo, C₁-C₈alkyl, C₁-C₈haloalkyl, cyano, hydroxy, C₁-C₈alkoxy, and amino.

Examples of representative substituted aryls include the following

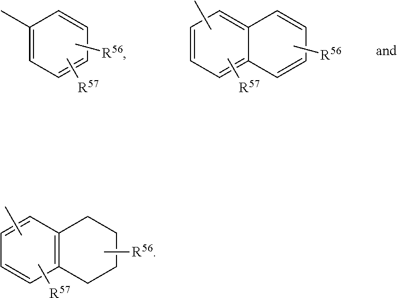

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁-C₈ alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

Other representative aryl groups having a fused heterocyclyl group include the following:

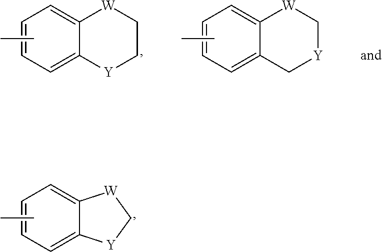

wherein each W is selected from C(R⁶⁶)₂, NR⁶⁶, O, and S; and each Y is selected from carbonyl, NR⁶⁶, O and S; and R⁶⁶ is independently hydrogen, C₁-C₈ alkyl, C₃-C₁₀ cycloalkyl, 4-10 membered heterocyclyl, C₆-C₁₀ aryl, and 5-10 membered heteroaryl.

“Fused aryl” refers to an aryl having two of its ring carbon in common with a second aryl or heteroaryl ring or with a carbocyclyl or heterocyclyl ring.

“Aralkyl” is a subset of alkyl and aryl, as defined herein, and refers to an optionally substituted alkyl group substituted by an optionally substituted aryl group.

“Heteroaryl” refers to a radical of a 5-10 membered monocyclic or bicyclic 4n+2 aromatic ring system (e.g., having 6 or 10 it electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following formulae:

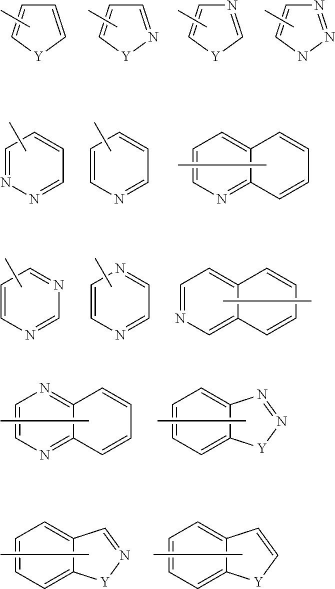

wherein each Y is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

“Heteroaralkyl” is a subset of alkyl and heteroaryl, as defined herein, and refers to an optionally substituted alkyl group substituted by an optionally substituted heteroaryl group.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Particular examples of heterocyclyl groups are shown in the following illustrative examples:

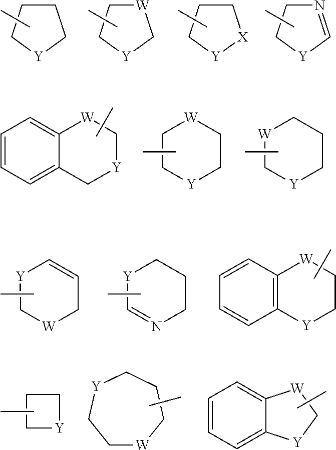

wherein each W is selected from CR⁶⁷, C(R⁶⁷)₂, NR⁶⁷, O, and S; and each Y is selected from NR⁶⁷, O, and S; and R⁶⁷ is independently hydrogen, C₁-C₈alkyl, C₃-C₁₀cycloalkyl, 4-10 membered heterocyclyl, C₆-C₁₀aryl, and 5-10-membered heteroaryl. These heterocyclyl rings may be optionally substituted with one or more groups selected from the group consisting of acyl, acylamino, acyloxy, alkoxy, alkoxycarbonyl, alkoxycarbonylamino, amino, substituted amino, aminocarbonyl (carbamoyl or amido), aminocarbonylamino, aminosulfonyl, sulfonylamino, aryl, aryloxy, azido, carboxyl, cyano, cycloalkyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.

“Acyl” refers to a radical —C(O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Acylamino” refers to a radical —NR²²C(O)R²³, where each instance of R²² and R²³ is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein, or R²² is an amino protecting group. Exemplary “acylamino” groups include, but are not limited to, formylamino, acetylamino, cyclohexylcarbonylamino, cyclohexylmethyl-carbonylamino, benzoylamino and benzylcarbonylamino. Particular exemplary “acylamino” groups are —NR²⁴C(O)—C₁-C₈ alkyl, —NR²⁴C(O)—(CH₂)ₜ(C₆-C₁₀ aryl), —NR²⁴C(O)—(CH₂)ₜ(5-10 membered heteroaryl), —NR²⁴C(O)—(CH₂)ₜ(C₃-C₁₀ cycloalkyl), and —NR²⁴C(O)—(CH₂)ₜ(4-10 membered heterocyclyl), wherein t is an integer from 0 to 4, and each R²⁴ independently represents H or C₁-C₈ alkyl. In certain embodiments, R²⁵ is H, C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; and R²⁶ is H, C₁-C₈ alkyl, substituted with halo or hydroxy; C₃-C₁₀ cycloalkyl, 4-10-membered heterocyclyl, C₆-C₁₀ aryl, arylalkyl, 5-10-membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxyl; provided at least one of R²⁵ and R²⁶ is other than H.

“Acyloxy” refers to a radical —OC(O)R²⁷, where R²⁷ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Representative examples include, but are not limited to, formyl, acetyl, cyclohexylcarbonyl, cyclohexylmethylcarbonyl, benzoyl, and benzylcarbonyl. In certain embodiments, R²⁸ is C₁-C₈ alkyl, substituted with halo or hydroxy; C₃-C₁₀ cycloalkyl, 4-10-membered heterocyclyl, C₆-C₁₀ aryl, arylalkyl, 5-10-membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e., with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary “substituted alkoxy” groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Amino” refers to the radical —NH₂.

“Substituted amino” refers to an amino group of the formula —N(R³⁸)₂ wherein R³⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, C₁-C₈ alkyl, C₃-C₈ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

“Azido” refers to the radical —N₃.

“Carbamoyl” or “amido” refers to the radical —C(O)NH₂.

“Substituted carbamoyl” or “substituted amido” refers to the radical —C(O)N(R⁶²)₂ wherein each R⁶² is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R⁶² is not a hydrogen. In certain embodiments, R⁶² is selected from H, C₁-C₈ alkyl, C₃-C₁₀ cycloalkyl, 4-10 membered heterocyclyl, C₆-C₁₀ aryl, aralkyl, 5-10 membered heteroaryl, and heteroaralkyl; or C₁-C₈ alkyl substituted with halo or hydroxy; or C₃-C₁₀ cycloalkyl, 4-10 membered heterocyclyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; provided that at least one R⁶² is other than H.

Exemplary “substituted carbamoyl” groups include, but are not limited to, —C(O) NR⁶⁴—C₁-C₈ alkyl, —C(O)NR⁶⁴—(CH₂)ₜ(C₆-C₁₀ aryl), —C(O)N⁶⁴—(CH₂)ₜ(5-10 membered heteroaryl), —C(O)NR⁶⁴—(CH₂)ₜ(C₃-C₁₀ cycloalkyl), and —C(O)NR⁶⁴—(CH₂)ₜ(4-10 membered heterocyclyl), wherein t is an integer from 0 to 4, each R⁶⁴ independently represents H or C₁-C₈ alkyl and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Carboxy” refers to the radical —C(O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Hydroxy” refers to the radical —OH.

“Nitro” refers to the radical —NO₂.

“Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Cycloalkenyl” refers to substituted or unsubstituted carbocyclyl group having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Fused cycloalkenyl” refers to a cycloalkenyl having two of its ring carbon atoms in common with a second aliphatic or aromatic ring and having its olefinic unsaturation located to impart aromaticity to the cycloalkenyl ring.

“Ethenyl” refers to substituted or unsubstituted —(C═C)—. “Ethylene” refers to substituted or unsubstituted —(C—C)—. “Ethynyl” refers to —(C≡C)—.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃+X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; wherein X is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14-membered heteroaryl, or two R^{cc} groups attached to a nitrogen atom are joined to form a 3-14-membered heterocyclyl or 5-14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is an amino protecting group (also referred to herein as a nitrogen protecting group). Amino protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14-membered heterocyclyl, C₆₋₁₄ aryl, and 5-14-membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Amino protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, amino protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Amino protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Amino protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other amino protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxyl)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on an sulfur atom is an sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

These and other exemplary substituents are described in more detail in the Detailed Description, Examples, and Claims. The invention is not intended to be limited in any manner by the above exemplary listing of substituents.

### OTHER DEFINITIONS

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

“Solvate” refers to forms of the compound that are associated with a solvent or water (also referred to as “hydrate”), usually by a solvolysis reaction. This physical association includes hydrogen bonding. Conventional solvents include water, ethanol, acetic acid, and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., ³H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O, and ¹³N, and would be useful in Positron Emission Topography (PET) studies for examining substrate receptor occupancy. All isotopic variants of the compounds provided herein, radioactive or not, are intended to be encompassed within the scope of the invention.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

### DETAILED DESCRIPTION OF CERTAIN EMBODIMENTS OF THE INVENTION

As generally described herein, the present invention provides neuroactive steroids comprising at least one ester or carbonate group at one or more positions 2, 6, 11, and/or 19 on the steroid scaffold, and designed, for example, to act as GABA modulators. In certain embodiments, such compounds are further envisioned to be useful as therapeutic agents for the mediation of anesthesia and/or sedation. The compounds of the present invention may, in certain additional embodiments, behave as “soft drugs,” e.g., compounds which are designed, upon administration, to exert a therapeutic effect in vivo prior to metabolism to less active or inactive compounds.

In one aspect, provided is a compound of Formula (I):

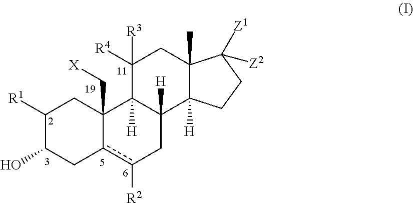

or a pharmaceutically acceptable salt, solvate, stereoisomer, tautomer, and/or isotopic variant thereof;
wherein:

X is hydrogen, halo, —CF₃, —CHF₂, —CH₂F, —NO₂, —CN, —SCN, —OR^{X}, —OC(═O)N(R^{X})₂, —SR^{X}, —SC(═O)N(R^{X})₂, —N(R^{X})₂, —NR^{X}C(═O)R^{X}, —NR^{X}C(═O)OR^{X}, —NR^{X}C(═O)N(R^{X})₂, —NR^{X}SO₂R^{X}, —C(═O)R^{X}, —C(═O)N(R^{X})₂, —S(═O)R^{X}, —S(═O)₂R^{X}, —SO₂N(R^{X})₂, or —OC(═O)R^{E1};

wherein R^{X} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{X} groups are joined to form a substituted or unsubstituted heterocyclic ring;

Z¹ is halo, —CN, —CH₂CN, —CH₂CF₃, —NO₂, —CH₂NO₂, —OR^{Z1b}, —CH₂OR^{Z1b}, —OC(═O)N(R^{Z1b})₂, —SR^{Z1b}, —N(R^{Z1b})₂, —N(OR^{Z1b})(R^{Z1b}), —NR^{Z1b}C(═O)R^{Z1b}, —NR^{Z1b}C(═O)OR^{Z1b}, —NR^{Z1b}C(═O)N(R^{Z1b})₂, —NR^{Z1b}SO₂R^{Z1b}, —C(═O)R^{Z1b}, —CH₂C(═O)R^{Z1b}, —C(═O)OR^{Z1b}, —CH₂C(═O)OR^{Z1b}, —C(═O)N(R^{Z1b})₂, —CH₂C(═O)N(R^{Z1b})₂, —S(═O)R^{Z1b}, —S(═O)₂R^{Z1b}, —SO₂N(R^{Z1b})₂, —P(═O)₂R^{Z1b}, —P(═O)₂OR^{Z1b}, —P(═O)(OR^{Z1b})₂, —P(═O)(R^{Z1b})₂, or —P(═O)(R^{Z1b})(OR^{Z1b}), —C(═O)CH₂OR^{Z1a}, or —C(═O)CH₂N(R^{Z1a})₂; and Z² is hydrogen or —OR^{Z2};

or Z¹ and Z² are joined to form a 3- to 6-membered substituted or unsubstituted heterocyclic ring; an oxo (═O); an oxime ═N(OR^{Z1b}); or an alkenyl group ═CH(Z³), wherein Z³ is —CF₃, —NO₂, —OR^{Z1b}, —C(═O)R^{Z1b}, —C(═O)OR^{Z1b}, or —C(═O)N(R^{Z1b})₂;

or Z¹ and Z² are joined to form an alkenyl group ═CH(CN), wherein CN is in the Z configuration;

wherein each instance of R^{Z1a} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a nitrogen protecting group when attached to a nitrogen atom, —C(═O)R^{Z1b}, —C(═O)OR^{Z1b}, —C(═O)N(R^{Z1b})₂, —C(═O)N(OR^{Z1b})(R^{Z1b}), —S(═O)₂R^{Z1b}, —S(═O)₂OR^{Z1b}, —P(═O)₂R^{Z1b}, —P(═O)₂OR^{Z1b}, —P(═O)(OR^{Z1b})₂, —P(═O)(R^{Z1b})₂, or —P(═O)(R^{Z1b})(OR^{Z1b}), or two R^{Z1a} groups are joined to form a substituted or unsubstituted heterocyclic ring or substituted or unsubstituted heteroaryl ring, and

wherein each instance of R^{Z1b} and R^{Z2} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{Z1b} groups are joined to form a substituted or unsubstituted heterocyclic or substituted or unsubstituted heteroaryl ring, or R^{Z1b} and R^{Z2} are joined to form a substituted or unsubstituted heterocyclic ring;

R¹, R², and R³ are independently selected from the group consisting of hydrogen or —OC(═O)R^{E1};

R⁴ is hydrogen, or R³ and R⁴ are joined to form an oxo (═O) group or an alkenyl group ═C(R^{A3})₂, wherein each instance of R^{A3} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or two R^{A3} groups are joined to form a substituted or unsubstituted carbocyclic ring or substituted or unsubstituted heterocyclic ring;

and

[symbol] represents a single or double bond, wherein if [symbol] is a single bond, then the C5 hydrogen is in the alpha or beta configuration;

provided at least one of R¹, R², R³, and Xa group of the formula —OC(═O)R^{E1};

wherein R^{E1} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or —OR^{E2}, and wherein R^{E2} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group.

In certain embodiments, [symbol] represents a double bond.

Alternatively, in certain embodiments, [symbol] represents a single bond, and the C5 hydrogen is in the alpha (down) or beta (up) configuration. In certain embodiments, [symbol] represents a single bond, and the C5 hydrogen is in the alpha (down) configuration. In certain embodiments, [symbol] represents a single bond, and the C5 hydrogen is in the beta (up) configuration.

### Group —OC(═O)R^{E1}

As generally described herein, compounds of the present invention comprise at least one ester or carbonate group of the formula —OC(═O)R^{E1} at positions 2, 6, 11, and/or 19 of the steroid scaffold, corresponding to groups R¹, R², R³, and X, respectively,

wherein R^{E1} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or —OR^{E2},

and wherein R^{E2} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group.

In certain embodiments, only one of R¹, R², R³, and X is a group of the formula —OC(═O)R^{E1}. For example, in certain embodiments, only R¹ is a group of the formula —OC(═O)R^{E1}. In certain embodiments, only R² is a group of the formula —OC(═O)R^{E1}. In certain embodiments, only R³ is a group of the formula —OC(═O)R^{E1}. In certain embodiments, only X is a group of the formula —OC(═O)R^{E1}.

In certain embodiments, at least two of R¹, R², R³, and X is a group of the formula —OC(═O)R^{E1}. For example, in certain embodiments, R¹ and R² are each independently a group of the formula —OC(═O)R^{E1}. In certain embodiments, R¹ and R³ are each independently a group of the formula —OC(═O)R^{E1}. In certain embodiments, R¹ and X are each independently a group of the formula —OC(═O)R^{E1}. In certain embodiments, R² and R³ are each independently a group of the formula —OC(═O)R^{E1}. In certain embodiments, R² and X are each independently a group of the formula —OC(═O)R^{E1}. In certain embodiments, R³ and X are each independently a group of the formula —OC(═O)R^{E1}.

In certain embodiments, at least three of R¹, R², R³, and X is a group of the formula —OC(═O)R^{E1}. For example, in certain embodiments R¹, R², and R³, are each independently a group of the formula —OC(═O)R^{E1}. In certain embodiments R¹, R², and X, are each independently a group of the formula —OC(═O)R^{E1}. In certain embodiments R¹, R³, and X, are each independently a group of the formula —OC(═O)R^{E1}. In certain embodiments R², R³, and X, are each independently a group of the formula —OC(═O)R^{E1}.

In certain embodiments, each of R¹, R², R³, and X are a group of the formula —OC(═O)R^{E1}.

In certain embodiments, each group of the formula —OC(═O)R^{E1} attached to the steroid scaffold is the same. In certain embodiments, at least two groups of the formula —OC(═O)R^{E1} attached to the steroid scaffold are the same, i.e., at least three or all groups of the formula —OC(═O)R^{E1} attached to the steroid scaffold are the same. In certain embodiments, each group of the formula —OC(═O)R^{E1} is different. In certain embodiments, at least two groups of the formula —OC(═O)R^{E1} attached to the steroid scaffold are different, e.g., at least three or all groups of the formula —OC(═O)R^{E1} attached to the steroid scaffold are different.

In certain embodiments, the group of —OC(═O)R^{E1} is an ester, wherein R^{E1} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl.

In certain embodiments, R^{E1} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{E1}C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), C₁₋₆ alkyl substituted with hydroxy groups (e.g., —CH₂OH, —CH(OH)₂), and C₁₋₆ alkyl substituted with alkoxy groups (e.g., —CH₂OCH₃ and —CH₂OCH₂CH₃).

In certain embodiments, R^{E1} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, R^{E1} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In certain embodiments, R^{E1} is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl.

In certain embodiments, R^{E1} is substituted or unsubstituted heterocyclyl, e.g., substituted or unsubstituted C₃₋₆ heterocyclyl, substituted or unsubstituted C₃₋₄ heterocyclyl, substituted or unsubstituted C₄₋₅ heterocyclyl, or substituted or unsubstituted C₅₋₆ heterocyclyl.

In certain embodiments, R^{E1} is substituted or unsubstituted aryl, e.g., substituted or unsubstituted phenyl.

In certain embodiments, R^{E1} is substituted or unsubstituted heteroaryl, e.g., optionally substituted C₅₋₆ heteroaryl.

In certain embodiments, the group of —OC(═O)R^{E1} is an carbonate, i.e., wherein R^{E1} is —OR^{E2}, to provide a group —OC(═O)OR^{E2}, wherein R^{E2} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group.

In certain embodiments, R^{E2} is hydrogen.

In certain embodiments, R^{E2} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{E2} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃).

In certain embodiments, R^{E2} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, R^{E2} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In certain embodiments, R^{E2} is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl.

In certain embodiments, R^{E2} is substituted or unsubstituted heterocyclyl, e.g., substituted or unsubstituted C₃₋₆ heterocyclyl, substituted or unsubstituted C₃₋₄ heterocyclyl, substituted or unsubstituted C₄₋₅ heterocyclyl, or substituted or unsubstituted C₅₋₆ heterocyclyl.

In certain embodiments, R^{E2} is substituted or unsubstituted aryl, e.g., substituted or unsubstituted phenyl.

In certain embodiments, R^{E2} is substituted or unsubstituted heteroaryl, e.g., optionally substituted C₅₋₆ heteroaryl.

In certain embodiments, R^{E2} is an oxygen protecting group.

### Additional Embodiments of Groups R¹, R², R³, and R⁴

As generally described herein, R¹, R², and R³ are independently selected from the group consisting of hydrogen or —OC(═O)R^{E1}; wherein R^{E1} is as defined herein, and R⁴ is hydrogen, or R³ and R⁴ are joined to form an oxo (═O) group or an alkenyl group ═C(R^{A3})₂, wherein each instance of R^{A3} is as defined herein.

In certain embodiments, R¹ is hydrogen. In certain embodiments, R¹ is —OC(═O)R^{E1}; wherein R^{E1} is as defined herein. In the instance where R¹ is —OC(═O)R^{E1}, the stereochemistry of the R¹ group is in the alpha (down) or beta (up) configuration. In certain embodiments, R¹ is in the alpha (down) configuration. In certain embodiments, R¹ is in the beta (up) configuration.

In certain embodiments, R¹ is an ester group of the formula —OC(═O)R^{E1}, e.g., wherein R^{E1} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In the instance, the stereochemistry of the R¹ group is in the alpha (down) or beta (up) configuration. In certain embodiments, R¹ is in the alpha (down) configuration. In certain embodiments, R¹ is in the beta (up) configuration.

In certain embodiments, R¹ is an carbonate group of the formula —OC(═O)OR^{E2}, e.g., wherein R^{E2} is, for example, hydrogen, or substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In the instance, the stereochemistry of the R¹ group is in the alpha (down) or beta (up) configuration. In certain embodiments, R¹ is in the alpha (down) configuration. In certain embodiments, R¹ is in the beta (up) configuration.

In certain embodiments, R² is hydrogen. In certain embodiments, R² is —OC(═O)R^{E1}; wherein R^{E1} is as defined herein. In the instance where R² is —OC(═O)R^{E1}, the stereochemistry of the R² group is in the alpha (down) or beta (up) configuration. In certain embodiments, R² is in the alpha (down) configuration. In certain embodiments, R² is in the beta (up) configuration.

In certain embodiments, R² is an ester group of the formula —OC(═O)R^{E1}, e.g., wherein R^{E1} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In the instance, the stereochemistry of the R² group is in the alpha (down) or beta (up) configuration. In certain embodiments, R² is in the alpha (down) configuration. In certain embodiments, R² is in the beta (up) configuration.

In certain embodiments, R² is an carbonate group of the formula —OC(═O)OR^{E2}, e.g., wherein R^{E2} is, for example, hydrogen, or substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In the instance, the stereochemistry of the R² group is in the alpha (down) or beta (up) configuration. In certain embodiments, R² is in the alpha (down) configuration. In certain embodiments, R² is in the beta (up) configuration.

In certain embodiments, R³ is hydrogen and R⁴ is hydrogen. In certain embodiments, R³ is —OC(═O)R^{E1} and R⁴ is hydrogen, wherein R^{E1} is as defined herein. In the instance where R³ is —OC(═O)R^{E1}, the stereochemistry of the R³ group is in the alpha (down) or beta (up) configuration. In certain embodiments, R³ is in the alpha (down) configuration. In certain embodiments, R³ is in the beta (up) configuration.

In certain embodiments, R³ is an ester group of the formula —OC(═O)R^{E1}, e.g., wherein R^{E1} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In the instance, the stereochemistry of the R³ group is in the alpha (down) or beta (up) configuration. In certain embodiments, R³ is in the alpha (down) configuration. In certain embodiments, R³ is in the beta (up) configuration.

In certain embodiments, R is an carbonate group of the formula —OC(═O)OR^{E2}, e.g., wherein R^{E2} is, for example, hydrogen, or substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In the instance, the stereochemistry of the R³ group is in the alpha (down) or beta (up) configuration. In certain embodiments, R³ is in the alpha (down) configuration. In certain embodiments, R³ is in the beta (up) configuration.

Alternatively, as generally described herein, R³ and R⁴ are joined to form an oxo (═O) group or an alkenyl group ═C(R^{A3})₂, wherein R^{A}3 is as defined herein. In certain embodiments, R³ and R⁴ are joined to form an oxo (═O) group. In certain embodiments, R³ and R⁴ are joined to form an alkenyl group ═C(R^{A3})₂, e.g., ═CHR^{A3}, provided in the Z- or E-configuration.

### Group X

Various compounds of Formula (I) are contemplated herein, i.e., wherein X is hydrogen, halo, —CF₃, —CHF₂, —CH₂F, —NO₂, —CN, —SCN, —OR^{X}, —OC(═O)N(R^{X})₂, —SR^{X}, —SC(═O)N(R^{X})₂, —N(R^{X})₂, —NR^{X}C(═O)R^{X}, —NR^{X}C(═O)OR^{X}, —NR^{X}C(═O)N(R^{X})₂, —NR^{X}SO₂R^{X}, —C(═O)R^{X}, —C(═O)N(R^{X})₂, —S(═O)R^{X}, —S(═O)₂R^{X}, or —SO₂N(R^{X})₂, wherein R^{X} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{X} groups are joined to form a substituted or unsubstituted heterocyclic ring, or X is —OC(═O)R^{E1}, wherein R^{E1} is as defined herein.

In certain embodiments, at least one instance of R^{X} is hydrogen or a protecting group, i.e., an oxygen protecting group when attached to an oxygen atom, a sulfur protecting group when attached to a sulfur atom, or a nitrogen protecting group when attached to a nitrogen atom. In certain embodiments, at least one instance of R^{X} is hydrogen.

In certain embodiments, at least one instance of R^{X} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{X}C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃).

In certain embodiments, at least one instance of R^{X} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, at least one instance of R^{X} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In certain embodiments, at least one instance of R^{X} is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl.

In certain embodiments, at least one instance of R^{X} is substituted or unsubstituted heterocyclyl, e.g., substituted or unsubstituted C₃₋₆ heterocyclyl, substituted or unsubstituted C₃₋₄ heterocyclyl, substituted or unsubstituted C₄₋₅ heterocyclyl, or substituted or unsubstituted C₅₋₆ heterocyclyl.

In certain embodiments, at least one instance of R^{X} is substituted or unsubstituted aryl, e.g., substituted or unsubstituted phenyl.

In certain embodiments, at least one instance of R^{X} is substituted or unsubstituted heteroaryl, e.g., optionally substituted C₅₋₆ heteroaryl.

In certain embodiments, two R^{X} groups are joined to form a substituted or unsubstituted heterocyclic ring, e.g., a substituted or unsubstituted piperidinyl, substituted or unsubstituted piperazinyl, or substituted or unsubstituted morpholinyl ring.

In certain embodiments, X is hydrogen.

In certain embodiments, X is halo, e.g., fluoro, bromo, chloro, or iodo. In certain embodiments, X is fluoro. In certain embodiments, X is bromo. In certain embodiments, X is chloro. In certain embodiments, X is iodo.

In certain embodiments, X is —CF₃, —CHF₂, or —CH₂F. In certain embodiments, X is —CF₃. In certain embodiments, X is —CHF₂. In certain embodiments, X is —CH₂F.

In certain embodiments, X is —NO₂.

In certain embodiments, X is —CN or —SCN. In certain embodiments, X is —CN. In certain embodiments, X is —SCN.

In certain embodiments, X is —SR^{X} or —SC(═O)N(R^{X})₂, wherein R^{X} is as defined herein.

In certain embodiments, X is —SH or —SR^{X}, e.g., wherein R^{X} is, for example, methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, X is —SC(═O)N(R^{X})₂, e.g., X is —SC(═O)NH₂ or —SC(═O)NHR^{X} wherein R^{X} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), or n-hexyl (C₆), or X is —SC(═O)N(R^{X})₂ wherein two R^{X} groups are joined to form a substituted or unsubstituted piperidinyl, substituted or unsubstituted piperazinyl, or substituted or unsubstituted morpholinyl ring.

In certain embodiments, X is —N(R^{X})₂, —NR^{X}C(═O)R^{X}, —NR^{X}C(═O)OR^{X}, —NR^{X}C(═O)N(R^{X})₂, or —NR^{X}SO₂R^{X}, wherein R^{X} is as defined herein.

In certain embodiments, X is —N(R^{X})₂, e.g., X is —NH₂ or —NHR^{X} wherein R^{X} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or X is —N(R^{X})₂ wherein the two R^{X} groups are joined to form a substituted or unsubstituted heterocyclic ring, e.g., substituted or unsubstituted piperidinyl, substituted or unsubstituted piperazinyl, or substituted or unsubstituted morpholinyl ring.

In certain embodiments, X is —NR^{X}C(═O)R^{X}, e.g., —NHC(═O)R^{X}, wherein R^{X} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, X is —NR^{X}C(═O)OR^{X}, e.g., —NHC(═O)OR^{X}, wherein R^{X} is, for example, methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, X is —NR^{X}C(═O)N(R^{X})₂, e.g., —NHC(═O)NH₂ or —NHC(═O)NHR^{X}, wherein R^{X} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or X is NHC(═O)N(R^{X})₂, wherein wherein the two R^{X} groups are joined to form a substituted or unsubstituted heterocyclic ring, e.g., substituted or unsubstituted piperidinyl, substituted or unsubstituted piperazinyl, or substituted or unsubstituted morpholinyl ring.

In certain embodiments, X is —NR^{X}SO₂R^{X}, e.g., —NHSO₂R^{X}, wherein R^{X} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, X is —C(═O)R^{X} or —C(═O)N(R^{X})₂, wherein R^{X} is as defined herein.

In certain embodiments, X is —C(═O)R^{X}, e.g., wherein R^{X} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, X is —C(═O)N(R^{X})₂, e.g., —C(═O)NH₂ or —C(═O)NHR^{X} wherein R^{X} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or X is —C(═O)N(R^{X})₂ wherein the two R^{X} groups are joined to form a substituted or unsubstituted heterocyclic ring, e.g., substituted or unsubstituted piperidinyl, substituted or unsubstituted piperazinyl, or substituted or unsubstituted morpholinyl ring.

In certain embodiments, X is —S(═O)R^{X}, —S(═O)₂R^{X}, or —SO₂N(R^{X})₂, wherein R^{X} is as defined herein.

In certain embodiments, X is —S(═O)R^{X}, e.g., wherein R^{X} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, X is —S(═O)₂R^{X}, e.g., wherein R^{X} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, X is —SO₂N(R^{X})₂, e.g., —SO₂NH₂ or —SO₂NHR^{X}, wherein R^{X} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), or substituted or unsubstituted phenyl, or X is —SO₂N(R^{X})₂ wherein the two R^{X} groups are joined to form a substituted or unsubstituted heterocyclic ring, e.g., substituted or unsubstituted piperidinyl, substituted or unsubstituted piperazinyl, or substituted or unsubstituted morpholinyl ring.

In certain embodiments, X is —OC(═O)R^{E1}; wherein R^{E1} is as defined herein.

In certain embodiments, X is an ester group of the formula —OC(═O)R^{E1}, e.g., wherein X is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, X is an carbonate group of the formula —OC(═O)OR^{E2}, e.g., wherein X is, for example, hydrogen, or substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

### Groups Z¹ and Z²

As generally described herein, Z¹ is halo, —CN, —CH₂CN, —CH₂CF₃, —NO₂, —CH₂NO₂, —OR^{Z1b}, —CH₂OR^{Z1b}, —OC(═O)N(R^{Z1b})₂, —SR^{Z1b}, —N(R^{Z1b})₂, —N(OR^{Z1b})(R^{Z1b}), —NR^{Z1b}C(═O)R^{Z1b}, —NR^{Z1b}C(═O)OR^{Z1b}, —NR^{Z1b}C(═O)N(R^{Z1b})₂, —NR^{Z1b}SOR^{Z1b}, —C(═O)R^{Z1b}, —CH₂C(═O)R^{Z1b}, —C(═O)OR^{Z1b}, —CH₂C(═O)OR^{Z1b}, —C(═O)N(R^{Z1b})₂, —CH₂C(═O)N(R^{Z1b})₂, —S(═O)R^{Z1b}, —S(═O)₂R^{Z1b}, —SO₂N(R^{Z1b})₂, —P(═O)₂R^{Z1b}, —P(═O)₂OR^{Z1b}, —P(═O)(OR^{Z1b})₂, —P(═O)(R^{Z1b})₂, or —P(═O)(R^{Z1b})(OR^{Z1b}), —C(═O)CH₂OR^{Z1a}, or —C(═O)CH₂N(R^{Z1a})₂; and Z² is hydrogen or —OR^{Z2};

or Z¹ and Z² are joined to form a 3- to 6-membered substituted or unsubstituted heterocyclic ring; an oxo (═O); an oxime ═N(OR^{Z1b}); or an alkenyl group ═CH(Z³), wherein Z³ is —CF₃, —CN, —NO₂, —OR^{Z1b}, —C(═O)R^{Z1b}, —C(═O)OR^{Z1b}, or —C(═O)N(R^{Z1b})₂;

wherein each instance of R^{Z1a} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a nitrogen protecting group when attached to a nitrogen atom, —C(═O)R^{Z1b}, —C(═O)OR^{Z1b}, —C(═O)N(R^{Z1b})₂, —C(═O)N(OR^{Z1b})(R^{Z1b}), —S(═O)₂R^{Z1b}, —S(═O)₂OR^{Z1b}, —P(═O)₂R^{Z1b}, —P(═O)₂OR^{Z1b}, —P(═O)(OR^{Z1b})₂, —P(═O)(R^{Z1b})₂, or —P(═O)(R^{Z1b})(OR^{Z1b}), or two R^{Z1a} groups are joined to form a substituted or unsubstituted heterocyclic ring or substituted or unsubstituted heteroaryl ring, and

wherein each instance of R^{Z1b} and R^{Z2} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{Z1b} groups are joined to form a substituted or unsubstituted heterocyclic or substituted or unsubstituted heteroaryl ring, or R^{Z1b} and R^{Z2} are joined to form a substituted or unsubstituted heterocyclic ring;

In certain embodiments, at least one instance of R^{Z1b} is hydrogen or a protecting group, i.e., an oxygen protecting group when attached to an oxygen atom, a sulfur protecting group when attached to a sulfur atom, or a nitrogen protecting group when attached to a nitrogen atom. In certain embodiments, at least one instance of R^{Z1b} is hydrogen. In certain embodiments, at least one instance of R^{Z1b} is a protecting group.

In certain embodiments, at least one instance of R^{Z1b} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{Z1b}C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃).

In certain embodiments, at least one instance of R^{Z1b} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, at least one instance of R^{Z1b} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In certain embodiments, at least one instance of R^{Z1b} is substituted or unsubstituted carbocyclyl, e.g., substituted or unsubstituted C₃₋₆ carbocyclyl, substituted or unsubstituted C₃₋₄carbocyclyl, substituted or unsubstituted C₄₋₅ carbocyclyl, or substituted or unsubstituted C₅₋₆ carbocyclyl.

In certain embodiments, at least one instance of R^{Z1b} is substituted or unsubstituted heterocyclyl, e.g., substituted or unsubstituted C₃₋₄ heterocyclyl, substituted or unsubstituted C₃₋₄ heterocyclyl, substituted or unsubstituted C₄₋₅ heterocyclyl, or substituted or unsubstituted C₅₋₆ heterocyclyl.

In certain embodiments, at least one instance of R^{Z1b} is substituted or unsubstituted aryl, e.g., substituted or unsubstituted phenyl.

In certain embodiments, at least one instance of R^{Z1b} is substituted or unsubstituted heteroaryl, e.g., optionally substituted C₅₋₆ heteroaryl.

Alternatively, in certain embodiments, two R^{Z1b} groups, e.g., for example attached to a nitrogen atom, are joined to form a substituted or unsubstituted heterocyclic ring, e.g., a substituted or unsubstituted piperidinyl, substituted or unsubstituted piperazinyl, or substituted or unsubstituted morpholinyl ring.

Alternatively, in certain embodiments, two R^{Z1b} groups, e.g., for example attached to a nitrogen atom, are joined to form a substituted or unsubstituted heteroaryl ring, e.g., a 5- to 6-membered heteroaryl ring.

In certain embodiments, R^{Z2} is hydrogen or an oxygen protecting group. In certain embodiments, R^{Z2} is hydrogen. In certain embodiments, R^{Z2} is an oxygen protecting group.

In certain embodiments, R^{Z2} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{Z2}C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃).

In certain embodiments, R^{Z2} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, R^{Z2} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In certain embodiments, R^{Z2} is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl.

In certain embodiments, R^{Z2} is substituted or unsubstituted heterocyclyl, e.g., substituted or unsubstituted C₃₋₆ heterocyclyl, substituted or unsubstituted C₃₋₄ heterocyclyl, substituted or unsubstituted C₄₋₅ heterocyclyl, or substituted or unsubstituted C₅₋₆ heterocyclyl.

In certain embodiments, R^{Z2} is substituted or unsubstituted aryl, e.g., substituted or unsubstituted phenyl.

In certain embodiments, R^{Z2} is substituted or unsubstituted heteroaryl, e.g., optionally substituted C₅₋₆ heteroaryl.

Alternatively, in certain embodiments, an R^{Z1b} group and R^{Z2} are joined to form a substituted or unsubstituted heterocyclic ring, e.g., a 5- to 6-membered substituted or unsubstituted heterocyclic ring.

In certain embodiments, Z¹ is halo, e.g., fluoro, chloro, bromo, or iodo; and Z² is hydrogen. In certain embodiments, Z¹ is fluoro. In certain embodiments, Z¹ is chloro. In certain embodiments, Z¹ is bromo. In certain embodiments, Z¹ is iodo.

In certain embodiments, Z¹ is —CN or —CH₂CN; and Z² is hydrogen or —OR^{Z2}. In certain embodiments, Z¹ is —CN. In certain embodiments, Z¹ is —CH₂CN. In these instances, in certain embodiments, Z² is hydrogen. In these instance, in other embodiments, Z² is —OR^{Z2}, e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —CH₂CF₃; and Z² is hydrogen or —OR^{Z2}. In this instance, in certain embodiments, Z² is hydrogen. In this instance, in other embodiments, Z² is —OR^{Z2}, e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —NO₂ or —CH₂NO₂; and Z² is hydrogen or —OR^{Z2}. In certain embodiments, Z¹ is —NO₂. In certain embodiments, Z¹ is —CH₂NO₂. In these instances, in certain embodiments, Z² is hydrogen. In these instances, in other embodiments, Z² is —OR^{Z2}, e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —OR^{Z1b} or —CH₂OR^{Z1b}; and Z² is hydrogen or —OR^{Z2} wherein R^{Z1b} and R^{Z2} are as defined herein.

In certain embodiments, Z¹ is —OR^{Z1b}, e.g., wherein R^{Z1b} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In this instance, in certain embodiments, Z² is hydrogen. In this instance, in other embodiments, Z² is —OR^{Z2}, e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or R^{Z1b} and R^{Z2} are joined to form a substituted or unsubstituted 5- to 6-membered heterocyclic ring.

In certain embodiments, Z¹ is —CH₂OR^{Z1b}, e.g., wherein R^{Z1b} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In these instances, in certain embodiments, Z² is hydrogen. In this instance, in other embodiments, Z² is —OR^{Z2}, e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or R^{Z1b} and R^{Z2} are joined to form a substituted or unsubstituted 5- to 6-membered heterocyclic ring.

In certain embodiments, Z¹ is —OC(═O)N(R^{Z1b})₂; e.g., wherein each instance of R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or the two R^{Z1b} groups are joined to form a substituted or unsubstituted 5- to 6-membered heterocyclic ring or 5- to 6-membered heteroaryl ring. In this instance, in certain embodiments, Z² is hydrogen. In this instance, in other embodiments, Z² is —OR^{Z2}, e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or R^{Z1b} and R^{Z2} are joined to form a substituted or unsubstituted 5- to 6-membered heterocyclic ring.

In certain embodiments, Z¹ is —SR^{Z1b}; e.g., wherein R^{Z1b} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In this instance, in certain embodiments, Z² is hydrogen. In certain embodiments, Z² is —OR^{Z2}, e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or R^{Z1b} and R^{Z2} are joined to form a substituted or unsubstituted 5-6-membered heterocyclic ring.

In certain embodiments, Z¹ is —N(R^{Z1b})₂, —N(OR^{Z1b})(R^{Z1b}), —NR^{Z1b}C(═O)R^{Z1b}, —NR^{Z1b}C(═O)OR^{Z1b}, —NR^{Z1b}C(═O)N(R^{Z1b})₂, or —NR^{Z1b}SOR^{Z1b}; and Z² is hydrogen or —OR^{Z2} wherein R^{Z1b} and R^{Z2} are as defined herein.

In certain embodiments, Z¹ is —N(R^{Z1b})₂; e.g., wherein each instance of R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or the two R^{Z1b} groups are joined to form a substituted or unsubstituted 5- to 6-membered heterocyclic ring or 5- to 6-membered heteroaryl ring. In this instance, in certain embodiments, Z² is hydrogen. In certain embodiments, Z² is —OR^{Z2}, e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —N(OR^{Z1b})(R^{Z1b}); e.g., wherein each instance of R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or the two R^{Z1b} groups are joined to form a substituted or unsubstituted 5- to 6-membered heterocyclic ring. In this instance, in certain embodiments, Z² is hydrogen. In certain embodiments, Z² is —OR^{Z2}, e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z is —NR^{Z1b}C(═O)R^{Z1b}; e.g., wherein each instance of R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or the two R^{Z1b} groups are joined to form a substituted or unsubstituted 5- to 6-membered heterocyclic ring. In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —NR^{Z1b}C(═O)OR^{Z1b}; e.g., wherein each instance of R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or the two R^{Z1b} groups are joined to form a substituted or unsubstituted 5- to 6-membered heterocyclic ring. In this instance, in certain embodiments, Z² is hydrogen. In certain embodiments, Z² is —OR^{Z2}, e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —NR^{Z1b}C(═O)N(R^{Z1b})₂; e.g., wherein each instance of R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or the two R^{Z1b} groups are joined to form a substituted or unsubstituted 5- to 6-membered heterocyclic ring, or two R^{Z1b} groups directly attached to the nitrogen atom are joined to form a 5- to 6-membered heteroaryl ring. In this instance, in certain embodiments, Z² is hydrogen. In certain embodiments, Z² is —OR^{Z2}, e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —NR^{Z1b}SO₂R^{Z1b}; e.g., wherein each instance of R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or the two R^{Z1b} groups are joined to form a substituted or unsubstituted 5- to 6-membered heterocyclic ring. In this instance, in certain embodiments, Z² is hydrogen. In certain embodiments, Z² is —OR^{Z2}, e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —C(═O)R^{Z1b} or —CH₂C(═O)R^{Z1b}, and Z² is hydrogen or —OR^{Z2}, wherein R^{Z1b} and R^{Z2} are as defined herein.

In certain embodiments, Z¹ is —C(═O)R^{Z1b}; e.g., wherein R^{Z1b} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z is —CH₂C(═O)R^{Z1b}; e.g., wherein R^{Z1b} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —C(═O)OR^{Z1b} or —CH₂C(═O)OR^{Z1b}; and Z² is hydrogen or —OR^{Z2}.

In certain embodiments, Z¹ is —C(═O)OR^{Z1b}; e.g., wherein R^{Z1b} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z is —CH₂C(═O)OR^{Z1b}; e.g., wherein R^{Z1b} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —C(═O)N(R^{Z1b})₂ or —CH₂C(═O)N(R^{Z1b})₂; and Z² is hydrogen or —OR^{Z2}.

In certain embodiments, Z¹ is —C(═O)N(R^{Z1b})₂; e.g., wherein each instance of R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or the two R^{Z1b} groups are joined to form a substituted or unsubstituted 5- to 6-membered heterocyclic ring, or two R^{Z1b} groups directly attached to the nitrogen atom are joined to form a 5- to 6-membered heteroaryl ring. In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z is —CH₂C(═O)N(R^{Z1b})₂; e.g., wherein each instance of R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or the two R^{Z1b} groups are joined to form a substituted or unsubstituted 5- to 6-membered heterocyclic ring, or two R^{Z1b} groups directly attached to the nitrogen atom are joined to form a 5- to 6-membered heteroaryl ring. In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —S(═O)R^{Z1b}, —S(═O)₂R^{Z1b}, or —SO₂N(R^{Z1b})₂; and Z² is hydrogen or —OR^{Z2}.

In certain embodiments, Z¹ is —S(═O)R^{Z1b}; e.g., wherein R^{Z1b} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or substituted or unsubstituted phenyl. In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —S(═O)₂R^{Z1b}; e.g., wherein R^{Z1b} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or substituted or unsubstituted phenyl. In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z is —SO₂N(R^{Z1b})₂; e.g., wherein each instance of R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or the two R^{Z1b} groups are joined to form a substituted or unsubstituted 5- to 6-membered heterocyclic ring, or two R^{Z1b} groups directly attached to the nitrogen atom are joined to form a 5- to 6-membered heteroaryl ring. In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —P(═O)₂R^{Z1b}, —P(═O)₂OR^{Z1b}, —P(═O)(OR^{Z1b})₂, —P(═O)(R^{Z1b})₂, or —P(═O)(R^{Z1b})(OR^{Z1b}); and Z² is hydrogen or —OR^{Z2}.

In certain embodiments, Z¹ is —P(═O)₂R^{Z1b}; e.g., wherein R^{Z1b} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or substituted or unsubstituted phenyl. In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —P(═O)₂OR^{Z1b}; e.g., wherein R^{Z1b} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or substituted or unsubstituted phenyl. In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —P(═O)(OR^{Z1b})₂; e.g., wherein each instance R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or substituted or unsubstituted phenyl. In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —P(═O)(R^{Z1b})₂; e.g., wherein each instance R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or substituted or unsubstituted phenyl. In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, Z¹ is —P(═O)(R^{Z1b})(OR^{Z1b}); e.g., wherein each instance R^{Z1b} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or substituted or unsubstituted phenyl. In this instance, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

As generally described herein, in certain embodiments, Z¹ is —C(═O)CH₂OR^{Z1a} or —C(═O)CH₂N(R^{Z1a})₂, wherein each instance of R^{Z1a} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a nitrogen protecting group when attached to a nitrogen atom, —C(═O)R^{Z1b}, —C(═O)OR^{Z1b}, —C(═O)N(R^{Z1b})₂, —C(═O)N(OR^{Z1b})(R^{Z1b}), —S(═O)₂R^{Z1b}, —S(═O)₂OR^{Z1b}, —P(═O)₂R^{Z1b}, —P(═O)₂OR^{Z1b}, —P(═O)(OR^{Z1b})₂, —P(═O)(R^{Z1b})₂, or —P(═O)(R^{Z1b})(OR^{Z1b}), or two R^{Z1a} groups are joined to form a substituted or unsubstituted heterocyclic ring or substituted or unsubstituted heteroaryl ring.

In certain embodiments, Z¹ is —C(═O)CH₂OR^{Z1a}. In certain embodiments, Z is —C(═O)CH₂N(R^{Z1a})₂. In these instances, in certain embodiments, Z² is hydrogen. In certain embodiments, Z² is —OR^{Z2}; e.g., wherein R^{Z2} is, for example, hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, at least one instance of R^{Z1a} is hydrogen.

In certain embodiments, at least one instance of R^{Z1a} is substituted or unsubstituted alkyl; e.g., substituted or unsubstituted C₁₋₆ alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{Z1a}C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃).

In certain embodiments, at least one instance of R^{Z1a} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, at least one instance of R^{Z1a} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In certain embodiments, at least one instance of R^{Z1a} is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl.

In certain embodiments, at least one instance of R^{Z1a} is substituted or unsubstituted heterocyclyl, e.g., substituted or unsubstituted C₃₋₆ heterocyclyl, substituted or unsubstituted C₃₋₄ heterocyclyl, substituted or unsubstituted C₄₋₅ heterocyclyl, or substituted or unsubstituted C₅₋₆ heterocyclyl.

In certain embodiments, at least one instance of R^{Z1a} is substituted or unsubstituted aryl, e.g., substituted or unsubstituted phenyl.

In certain embodiments, at least one instance of R^{Z1a} is substituted or unsubstituted heteroaryl, e.g., optionally substituted C₅₋₆ heteroaryl.

In certain embodiments, at least one instance of R^{Z1a} is a protecting group, e.g., an oxygen protecting group when attached to an oxygen atom, or a nitrogen protecting group when attached to a nitrogen atom.

In certain embodiments, at least one instance of R^{Z1a} is —C(═O)R^{Z1b}, —C(═O)OR^{Z1b}, —C(═O)N(R^{Z1b})₂, or —C(═O)N(OR^{Z1b})(R^{Z1b}), wherein R^{Z1b} is as defined herein. In certain embodiments, at least one instance of R^{Z1a} is —C(═O)R^{Z1b}. In certain embodiments, at least one instance of R^{Z1a} is —C(═O)OR^{Z1b}. In certain embodiments, at least one instance of R^{Z1a} is —C(═O)N(R^{Z1b})₂. In certain embodiments, at least one instance of R^{Z1a} is —C(═O)N(OR^{Z1b})(R^{Z1b}).

In certain embodiments, at least one instance of R^{Z1a} is —S(═O)₂R^{Z1b}, or —S(═O)₂OR^{Z1b}, wherein R^{Z1b} is as defined herein. In certain embodiments, at least one instance of R^{Z1a} is —S(═O)₂R^{Z1b}. In certain embodiments, at least one instance of R^{Z1a} is —S(═O)₂OR^{Z1b}.

In certain embodiments, at least one instance of R^{Z1a} is —P(═O)₂R^{Z1b}, —P(═O)₂OR^{Z1b}, —P(═O)(OR^{Z1b})₂, —P(═O)(R^{Z1b})₂, or —P(═O)(R^{Z1b})(OR^{Z1b}), wherein R^{Z1b} is as defined herein. In certain embodiments, at least one instance of R^{Z1a} is —P(═O)₂R^{Z1b}. In certain embodiments, at least one instance of R^{Z1a} is —P(═O)₂OR^{Z1b}. In certain embodiments, at least one instance of R^{Z1a} is —P(═O)(OR^{Z1b})₂. In certain embodiments, at least one instance of R^{Z1a} is —P(═O)(R^{Z1b})₂. In certain embodiments, at least one instance of R^{Z1a} is —P(═O)(R^{Z1b})(OR^{Z1b}).

Alternatively, in certain embodiments, two R^{Z1a} groups, e.g., for example attached to a nitrogen atom, are joined to form a substituted or unsubstituted heterocyclic ring, e.g., a substituted or unsubstituted piperidinyl, substituted or unsubstituted piperazinyl, or substituted or unsubstituted morpholinyl ring.

Alternatively, in certain embodiments, two R^{Z1a} groups, e.g., for example attached to a nitrogen atom, are joined to form a substituted or unsubstituted heteroaryl ring, e.g., a 5- to 6-membered heteroaryl ring.

Alternatively, as generally described herein, Z¹ and Z² are joined to form a 3- to 6-membered substituted or unsubstituted heterocyclic ring. For example, in certain embodiments, Z¹ and Z² are joined to form a 3-membered substituted or unsubstituted oxiranyl ring.

Alternatively, as generally described herein, Z¹ and Z² are joined to form an oxo (═O) group.

Alternatively, as generally described herein, Z¹ and Z² are joined to form an oxime ═N(OR^{Z1b}), wherein R^{Z1b} is as defined herein. The oxime group ═N(OR^{Z1b}) may be provided in the Z or E configuration as depicted below.

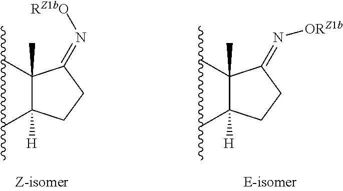

In certain embodiments, Z¹ and Z² are joined to form an oxime ═N(OR^{Z1b}) in the Z-configuration. In other embodiments, Z¹ and Z² are joined to form an oxime ═N(OR^{Z1b}) in the E-configuration.

Alternatively, as generally described herein, Z¹ and Z² are joined to form an alkenyl group ═CH(Z³), wherein Z³ is —CF₃, —NO₂, —OR^{Z1b}, —C(═O)R^{Z1b}, —C(═O)OR^{Z1b}, or —C(═O)N(R^{Z1b})₂, and wherein R^{Z1b} is as defined herein. Similar to the oxime, the alkenyl group ═CH(Z³) may be provided in the E or Z configuration as depicted below.

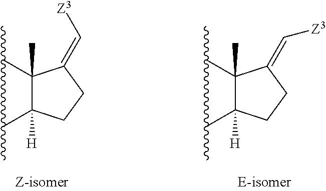

In certain embodiments, Z¹ and Z² are joined to form an alkenyl group ═CH(Z³) in the Z-configuration. In other embodiments, Z¹ and Z² are joined to form an alkenyl group ═CH(Z³) in the E-configuration.

In certain embodiments, Z¹ and Z² are joined to form the group ═CHCF₃. In certain embodiments, this group is in the E-configuration. In certain embodiments, this group is in the Z-configuration.

In certain embodiments, Z¹ and Z² are joined to form the group ═CHCN in the Z-configuration.

In certain embodiments, Z¹ and Z² are joined to form the group ═CHNO₂. In certain embodiments, this group is in the E-configuration. In certain embodiments, this group is in the Z-configuration.

In certain embodiments, Z¹ and Z² are joined to form the group ═CHOR^{Z1b}, wherein R^{Z1b} is as defined herein. In certain embodiments, this group is in the E-configuration. In certain embodiments, this group is in the Z-configuration.

In certain embodiments, Z¹ and Z² are joined to form the group ═CHC(═O)R^{Z1b} wherein R^{Z1b} is as defined herein. In certain embodiments, this group is in the E-configuration. In certain embodiments, this group is in the Z-configuration.

In certain embodiments, Z¹ and Z² are joined to form the group ═CHC(═O)OR^{Z1b}, wherein R^{Z1b} is as defined herein. In certain embodiments, this group is in the E-configuration. In certain embodiments, this group is in the Z-configuration.

In certain embodiments, Z¹ and Z² are joined to form the group ═CHC(═O)N(R^{Z1b})₂, wherein R^{Z1b} is as defined herein. In certain embodiments, this group is in the E-configuration. In certain embodiments, this group is in the Z-configuration.

The reduced form of the above described alkenyl group ═CH(Z³) is also contemplated, e.g., a group of formula —CH₂Z³, to provide a substituted alkyl group selected from the group consisting of —CH₂CF₃, —CH₂CN, —CH₂NO₂, —CH₂OR^{Z1b}, —CH₂C(═O)R^{Z1b}, —CH₂C(═O)OR^{Z1b}, and —CH₂C(═O)N(R^{Z1b})₂, wherein R^{Z1b} is as defined herein.

In any of the above instances, the stereochemistry of the Z¹ group may be provided in the alpha (down) or beta (up) configuration, and the stereochemistry of the Z² group is provided in the opposite configuration, i.e., in the beta (up) or alpha (down) configuration, respectively. In certain embodiments, Z² is in the alpha (down) configuration, and Z¹ is in the beta (up) configuration. In other embodiments, Z¹ is in the alpha (down) configuration, and Z² is in the beta (up) configuration.

### Additional Embodiments of Compounds of the Present Invention

Combinations of the above described embodiments are further contemplated.

In certain embodiments of Formula (I), provided is a compound of Formula (I-a), (I-b), or (I-c):

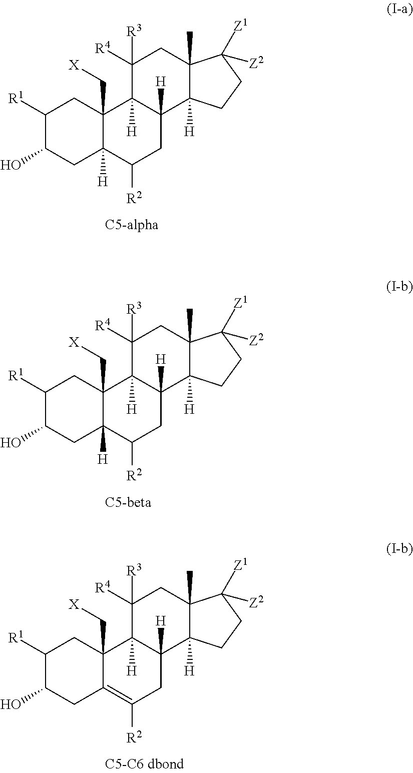

or a pharmaceutically acceptable salt, solvate, stereoisomer, tautomer, and/or isotopic variant thereof; wherein X, R¹, R², R³, R⁴, Z¹, and Z² are as defined herein.

In certain embodiments of Formula (I-a), Z¹ is provided in the alpha (down configuration) or beta (up) configuration to provide a compound of Formula (I-a1) or (I-a2), respectively:

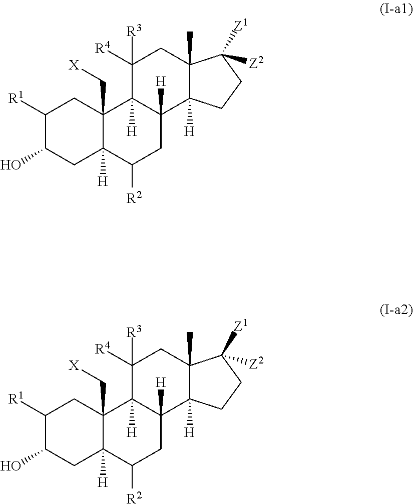

or a pharmaceutically acceptable salt, solvate, stereoisomer, tautomer, and/or isotopic variant thereof; wherein X, R¹, R², R³, R⁴, Z¹, and Z² are as defined herein.

In certain embodiments of Formula (I-a2), the compound is

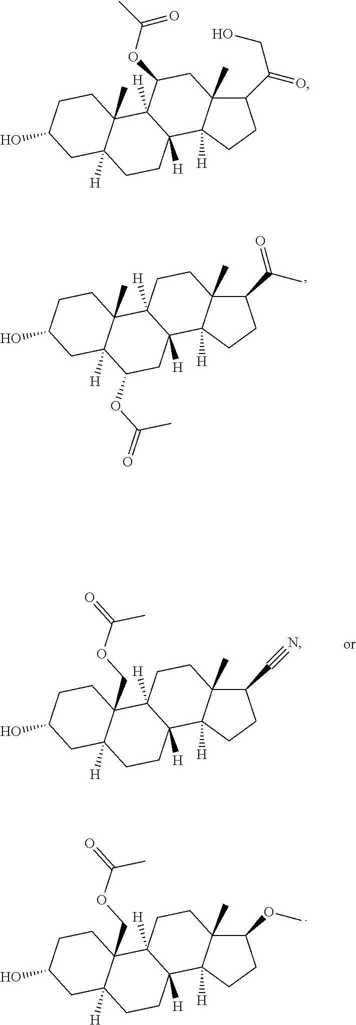 [SMILES: CC(=O)O[C@H]1C[C@]2(C)C(C(=O)CO)CC[C@H]2[C@@H]2CC[C@H]3C[C@H](O)CC[C@]3(C)[C@H]21 ; CC(=O)O[C@H]1C[C@@H]2[C@H](CC[C@]3(C)[C@@H](C(C)=O)CC[C@@H]23)[C@@]2(C)CC[C@@H](O)C[C@H]12 ; CC(=O)OC[C@]12CC[C@@H](O)C[C@@H]1CC[C@H]1[C@@H]3CC[C@H](C#N)[C@@]3(C)CC[C@@H]12 ; CO[C@H]1CC[C@H]2[C@@H]3CC[C@H]4C[C@H](O)CC[C@]4(COC(C)=O)[C@H]3CC[C@]12C]

In certain embodiments of Formula (I-b), Z¹ is provided in the alpha (down configuration) or beta (up) configuration to provide a compound of Formula (I-b1) or (I-b2), respectively:

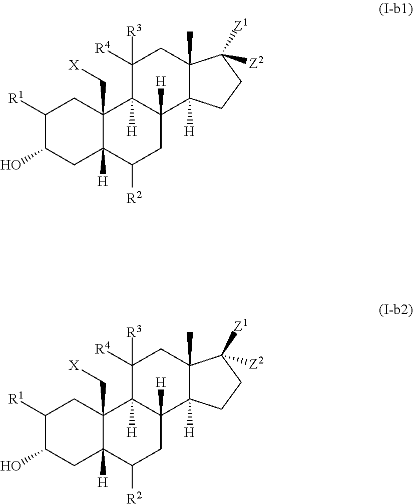

or a pharmaceutically acceptable salt, solvate, stereoisomer, tautomer, and/or isotopic variant thereof; wherein X, R¹, R², R³, R⁴, Z¹, and Z² are as defined herein.

In certain embodiments of Formula (I-c), Z¹ is provided in the alpha (down configuration) or beta (up) configuration to provide a compound of Formula (I-b1) or (I-b2), respectively:

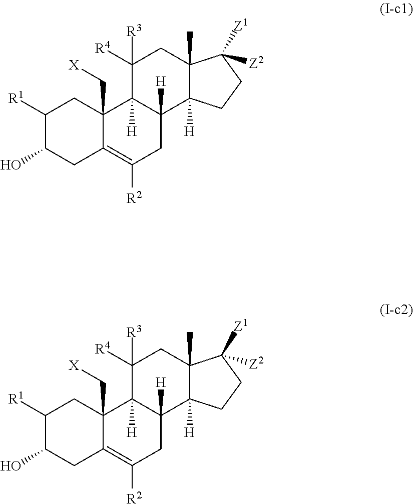

or a pharmaceutically acceptable salt, solvate, stereoisomer, tautomer, and/or isotopic variant thereof; wherein X, R¹, R², R³, R⁴, Z¹, and Z² are as defined herein.

In other embodiments of Formula (I), wherein Z¹ is alpha (down) or beta (up), provided is a compound of Formula (I-d) or (I-g):

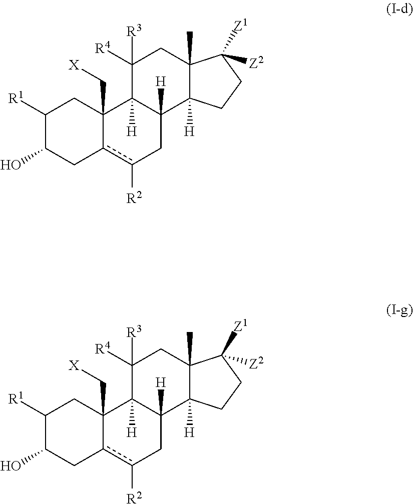

or a pharmaceutically acceptable salt, solvate, stereoisomer, tautomer, and/or isotopic variant thereof; wherein X, R¹, R², R³, R⁴, Z¹, and Z² are as defined herein.

In certain embodiments of Formula (I-g), wherein Z² is hydrogen and Z¹ is a substituted alkyl group of the formula —CH₂(Z³), provided is a compound of Formula (I-h):

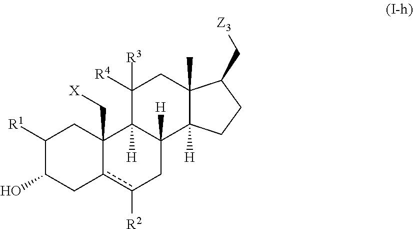

or a pharmaceutically acceptable salt, solvate, stereoisomer, tautomer, and/or isotopic variant thereof; wherein X, R¹, R², R³, R⁴, and Z³ are as defined herein. In certain embodiments, Z³ is —CF₃, —CN, or —NO₂.

In certain embodiments of Formula (I-h), the following isomers of Formula (I-h1), (I-h2), (I-h3), are specifically contemplated:

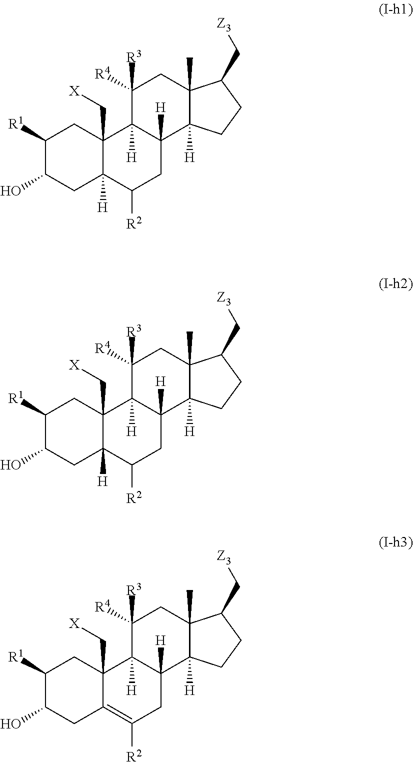

or a pharmaceutically acceptable salt, solvate, stereoisomer, tautomer, and/or isotopic variant thereof; wherein X, R¹, R², R³, R⁴, and Z³ are as defined herein.

In certain embodiments of Formula (I-g), wherein Z² is hydrogen, and Z¹ is beta-NO₂, OMe, or CN provided is a compound of Formula (I-j^{a}), (I-j^{b}), or (I-j^{c}):

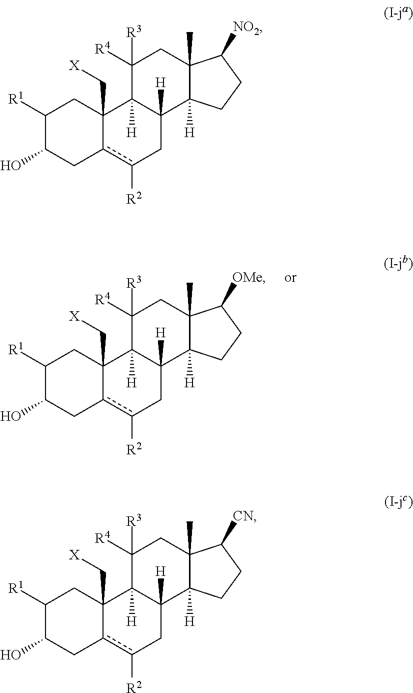

or a pharmaceutically acceptable salt, solvate, stereoisomer, tautomer, and/or isotopic variant thereof; wherein X, R¹, R², R³, and R⁴ are as defined herein.

In certain embodiments of Formula (I-j), the following isomers of Formula (I-j1), (I-j2), (I-j3), are specifically contemplated, wherein Z¹ is NO₂, OMe, or CN:

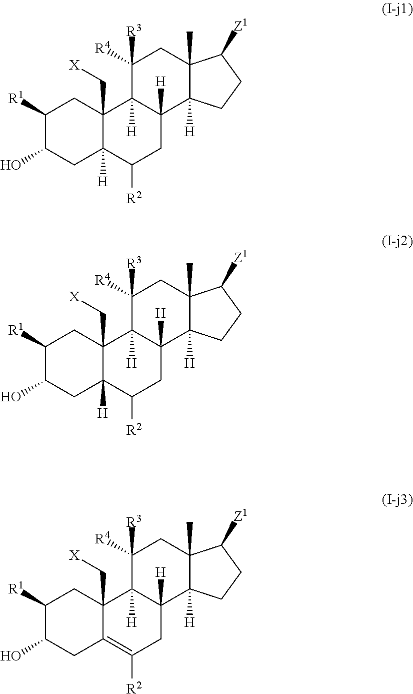

or a pharmaceutically acceptable salt, solvate, stereoisomer, tautomer, and/or isotopic variant thereof; wherein X, R¹, R², R³, and R⁴ are as defined herein.

In certain embodiments of Formula (I-g), wherein Z₁ and Z₂ are joined to form an oxo (═O), oxime ═N(OR^{Z3}), or an alkenyl group ═CH(Z³), provided is a compound of Formula (I-k), (I-m), or (I-n) respectively:

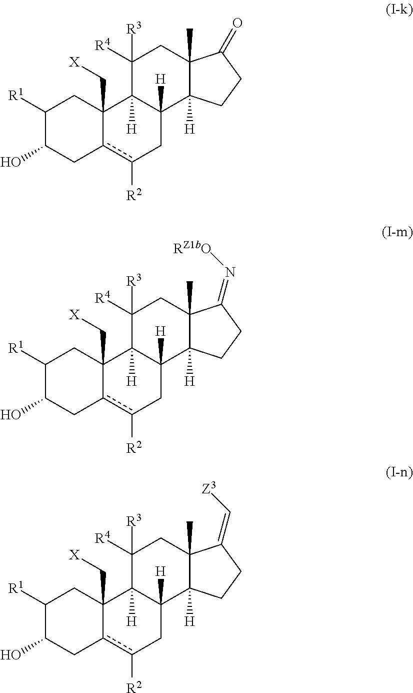

or a pharmaceutically acceptable salt, solvate, stereoisomer, tautomer, and/or isotopic variant thereof; wherein X, R¹, R², R³, R⁴, R^{Z1b}, and Z³ are as defined herein. In certain embodiments, Z³ is —CF₃, —CN, or —NO₂.

In certain embodiments of Formula (I-k), the following isomers of Formula (I-k1), (I-k2), (I-k3), are specifically contemplated:

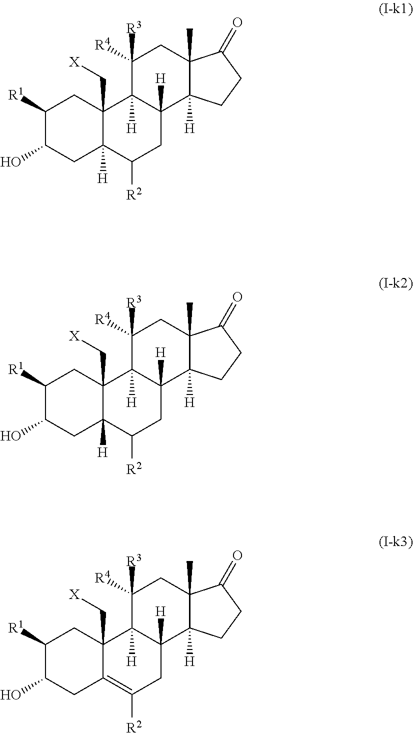

or a pharmaceutically acceptable salt, solvate, stereoisomer, tautomer, and/or isotopic variant thereof; wherein X, R¹, R², R³, and R⁴, are as defined herein.

In certain embodiments of Formula (I-m), the following isomers of Formula (I-m1), (I-m2), (I-m3), are specifically contemplated:

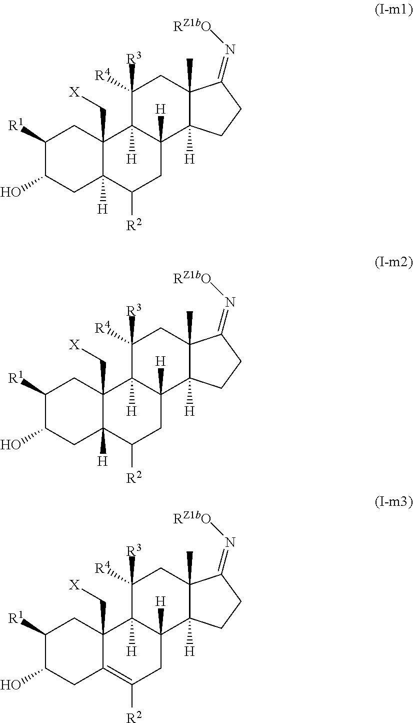

or a pharmaceutically acceptable salt, solvate, stereoisomer, tautomer, and/or isotopic variant thereof; wherein X, R¹, R², R³, R⁴, and R^{Z1b} are as defined herein.

In certain embodiments of Formula (I-n), the following isomers of Formula (I-n1), (I-n2), (I-n3), are specifically contemplated:

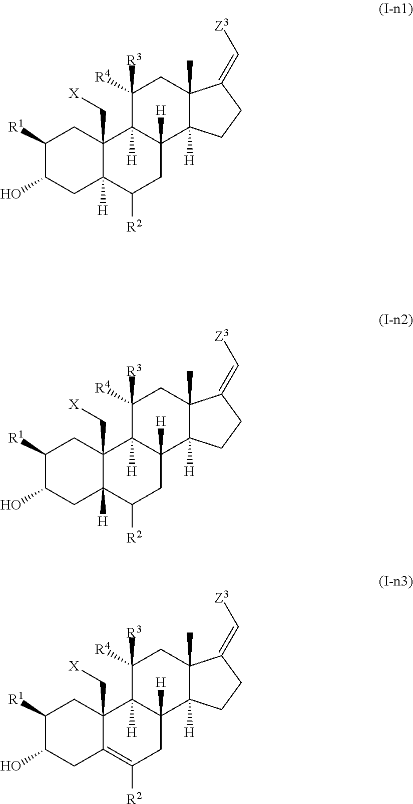

or a pharmaceutically acceptable salt, solvate, stereoisomer, tautomer, and/or isotopic variant thereof; wherein X, R¹, R², R³, R⁴, and Z³ are as defined herein.

In certain embodiments of Formula (I-n1), the compound of Formula (II-n1) is specifically contemplated:

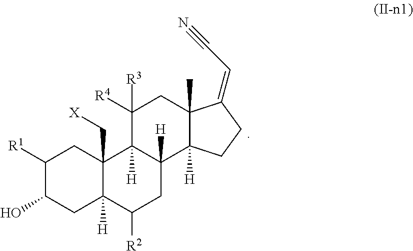

In certain embodiments of Formula (II-n1), the compound is

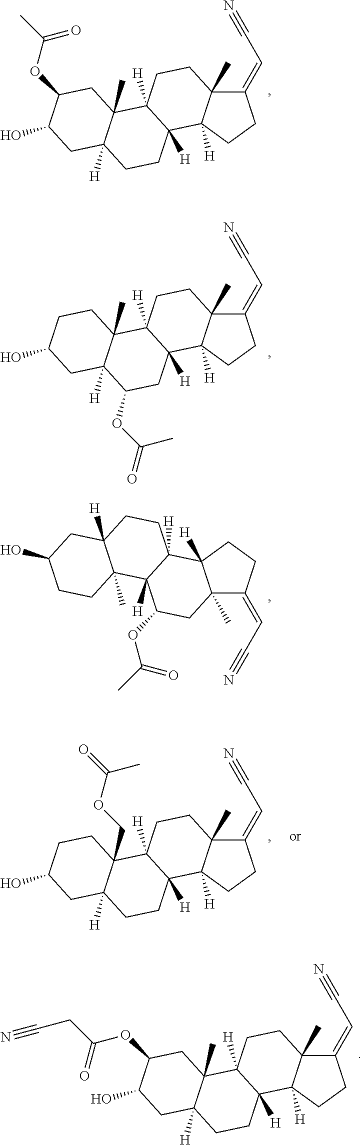

In certain embodiments of Formula (I-n2), the compound of Formula (II-n2) is specifically contemplated:

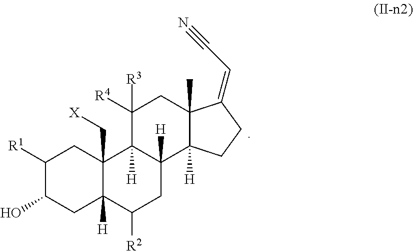

In certain embodiments of Formula (I-n2), the compound is

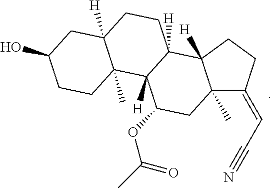 [SMILES: CC(=O)O[C@H]1C[C@]2(C)/C(=C\C#N)CC[C@H]2[C@@H]2CC[C@@H]3C[C@H](O)CC[C@]3(C)[C@H]21]

In certain embodiments of Formula (I-g), wherein Z² is hydrogen, and Z¹ is beta-C(═O)CH₂OR^{Z1a} or —C(═O)CH₂N(R^{Z1a})₂, provided is a compound of Formula (I-o) and (I-p):

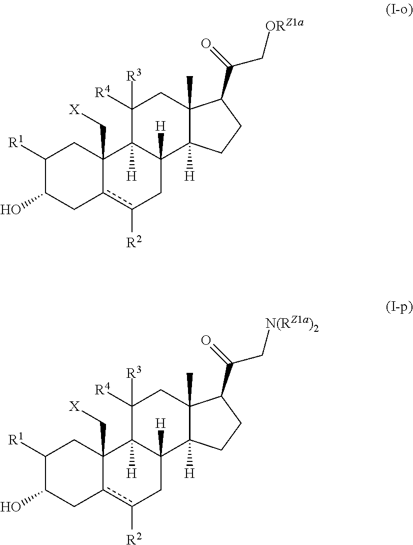

or a pharmaceutically acceptable salt, solvate, stereoisomer, tautomer, and/or isotopic variant thereof; wherein X, R¹, R², R³, R⁴, and R^{Z1a} are as defined herein.

In certain embodiments of Formula (I-o), the follow isomers of Formula (I-o1), (i-o2), (I-o3), are specifically contemplated:

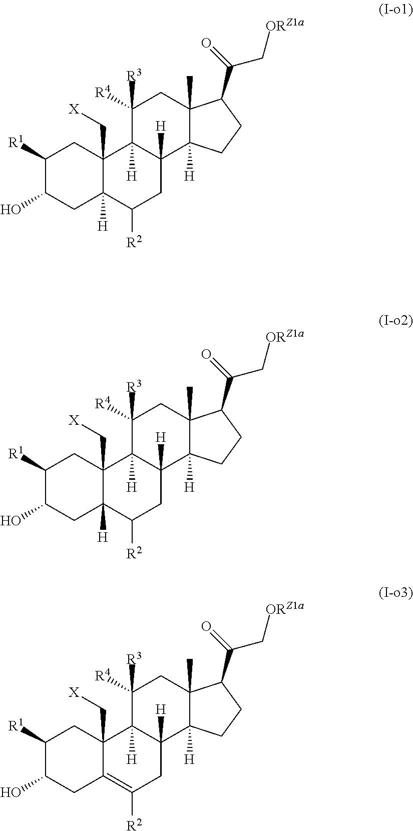

or a pharmaceutically acceptable salt, solvate, stereoisomer, tautomer, and/or isotopic variant thereof; wherein X, R¹, R², R³, R⁴, and R^{Z1a} are as defined herein.

In certain embodiments of Formula (I-o1), the compound of Formula (II-o) is specifically contemplated:

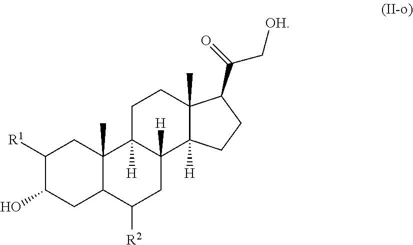

In certain embodiments of Formula (II-o), the compound is

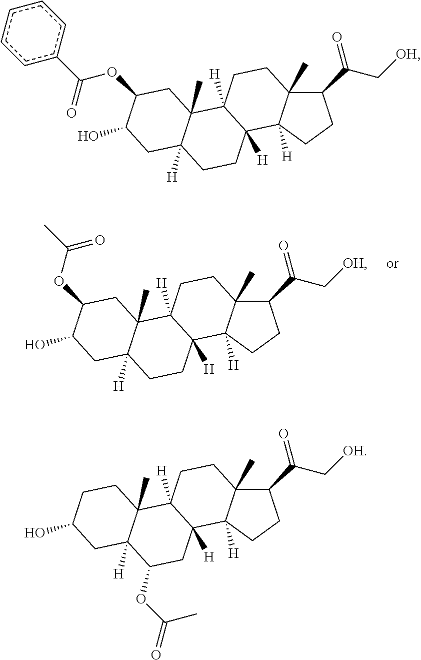

In certain embodiments of Formula (I-p), the following isomers of Formula (I-p1), (I-p2), (I-p3), are specifically contemplated:

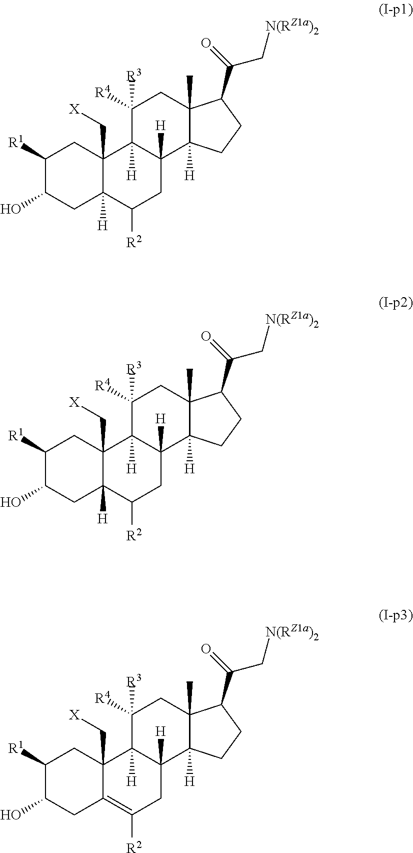

or a pharmaceutically acceptable salt, solvate, stereoisomer, tautomer, and/or isotopic variant thereof; wherein X, R¹, R², R³, R⁴, and R^{Z1a} are as defined herein.

Furthermore, as is understood from the above and below description, one or more groups of the formula —OC(═O)R^{E1}, as described herein, are attached to the compound of Formula (I) at one or more positions 2, 6, 11, 17, or 19, e.g., as depicted below:

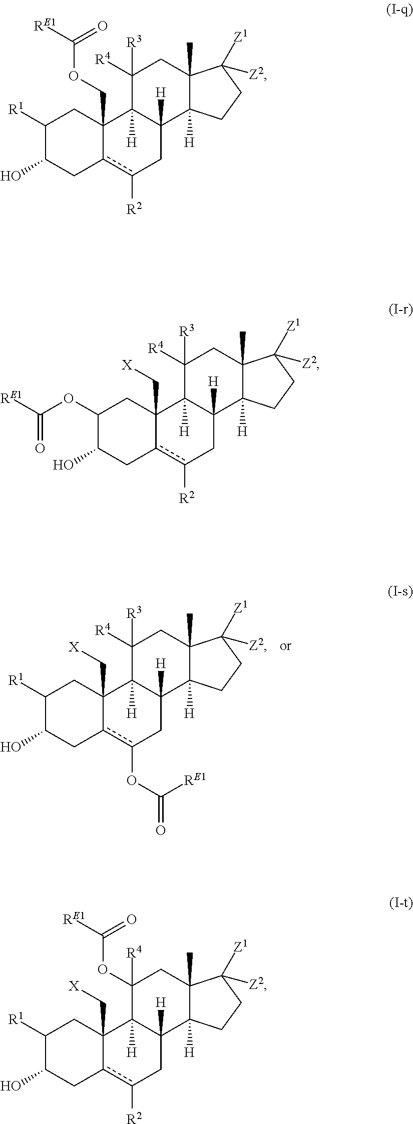

or a pharmaceutically acceptable salt, solvate, stereoisomer, tautomer, and/or isotopic variant thereof, wherein R¹, R², R³, R⁴, Z¹, Z², and R^{E1} are as defined herein.

In certain embodiments, Z¹ and Z² are joined to form an alkenyl group ═CH(CN), wherein CN is in the Z configuration. In certain embodiments, R¹, R², R³, and R⁴ are each H. In some embodiments, R^{E1} is —CH₃, —CH₂CN, or phenyl. In some embodiments, R^{E1} is —CH₃.

### Pharmaceutical Compositions

In another aspect, the invention provides a pharmaceutical composition comprising a compound of the present invention and a pharmaceutically acceptable excipient, e.g., a composition suitable for injection, such as for intravenous (IV) administration.

Pharmaceutically acceptable excipients include any and all diluents or other liquid vehicles, dispersion or suspension aids, surface active agents, isotonic agents, preservatives, lubricants and the like, as suited to the particular dosage form desired, e.g., injection. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

For example, injectable preparations, such as sterile injectable aqueous suspensions, can be formulated according to the known art using suitable dispersing or wetting agents and suspending agents. Exemplary excipients that can be employed include, but are not limited to, water, sterile saline or phosphate-buffered saline, or Ringer's solution.

In certain embodiments, the pharmaceutical composition further comprises a cyclodextrin derivative. The most common cyclodextrins are α-, β- and γ-cyclodextrins consisting of 6, 7 and 8 α-1,4-linked glucose units, respectively, optionally comprising one or more substituents on the linked sugar moieties, which include, but are not limited to, substituted or unsubstituted methylated, hydroxyalkylated, acylated, and sulfoalkylether substitution. In certain embodiments, the cyclodextrin is a sulfoalkyl ether β-cyclodextrin, e.g., for example, sulfobutyl ether β-cyclodextrin, also known as Captisol®. See, e.g., U.S. Pat. No. 5,376,645. In certain embodiments, the composition comprises hexapropyl-β-cyclodextrin. In a more particular embodiment, the composition comprises hexapropyl-β-cyclodextrin (10-50% in water).

The injectable composition can be sterilized, for example, by filtration through a bacterial-retaining filter, or by incorporating sterilizing agents in the form of sterile solid compositions which can be dissolved or dispersed in sterile water or other sterile injectable medium prior to use.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, response of the individual patient, the severity of the patient's symptoms, and the like.

When used to prevent the onset of a central nervous system (CNS)-disorder, the compounds provided herein will be administered to a subject at risk for developing the condition, typically on the advice and under the supervision of a physician, at the dosage levels described below. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compostions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pre-filled, pre-measured ampules or syringes of the liquid compositions. In such compositions, the compound is usually a minor component (from about 0.1% to about 50% by weight or preferably from about 1% to about 40% by weight) with the remainder being various vehicles or carriers and processing aids helpful for forming the desired dosing form.

Injection dose levels range from about 0.1 mg/kg/hour to at least 10 mg/kg/hour, all for from about 1 to about 120 hours and from about 24 to about 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient. An exemplary composition may be, for example, dissolved or suspended in a buffered sterile saline injectable aqueous medium to a concentration of approximately 5 mg/mL.

The compounds provided herein can be administered as the sole active agent, or they can be administered in combination with other active agents. In one aspect, the present invention provides a combination of a compound of the present invention and another pharmacologically active agent. Administration in combination can proceed by any technique apparent to those of skill in the art including, for example, separate, sequential, concurrent, and alternating administration.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Methods of Use and Treatment

Earlier studies (see, e.g., Gee et al., European Journal of Pharmacology, 136:419-423 (1987)) demonstrated that certain 3α-hydroxylated steroids are orders of magnitude more potent as modulators of the GABA receptor complex (GRC) than others had reported (see, e.g., Majewska et al., Science 232:1004-1007 (1986); Harrison et al., J Pharmacol. Exp. Ther. 241:346-353 (1987)). Majewska et al. and Harrison et al. taught that 3α-hydroxylated-5-reduced steroids are only capable of much lower levels of effectiveness. In vitro and in vivo experimental data have now demonstrated that the high potency of these steroids allows them to be therapeutically useful in the modulation of brain excitability via the GRC (see, e.g., Gee et al., European Journal of Pharmacology, 136:419-423 (1987); Wieland et al., Psychopharmacology 118(1):65-71 (1995)).

Various synthetic steroids have also been prepared as neuroactive steroids. See, for example, U.S. Pat. No. 5,232,917, which discloses neuroactive steroid compounds useful in treating stress, anxiety, insomnia, seizure disorders, and mood disorders, that are amenable to GRC-active agents, such as depression, in a therapeutically beneficial manner. Furthermore, it has been previously demonstrated that these steroids interact at a unique site on the GRC which is distinct from other known sites of interaction (e.g., barbiturates, benzodiazepines, and GABA) where therapeutically beneficial effects on stress, anxiety, sleep, mood disorders and seizure disorders have been previously elicited (see, e.g., Gee, K. W. and Yamamura, H. I., “Benzodiazepines and Barbiturates: Drugs for the Treatment of Anxiety, Insomnia and Seizure Disorders,” in Central Nervous System Disorders, Horvell, ed., Marcel-Dekker, New York (1985), pp. 123-147; Lloyd, K. G. and Morselli, P. L., “Psychopharmacology of GABAergic Drugs,” in Psychopharmacology: The Third Generation of Progress, H. Y. Meltzer, ed., Raven Press, N.Y. (1987), pp. 183-195; and Gee et al., European Journal of Pharmacology, 136:419-423 (1987). These compounds are desirable for their duration, potency, and oral activity (along with other forms of administration).

As generally described herein, the present invention is directed to certain neuroactive steroids comprising at least one ester or carbonate group at one or more positions 2, 6, 11, and/or 19 on the steroid scaffold, and designed, for example, to act as GABA modulators. In certain embodiments, such compounds are envisioned to be useful as therapeutic agents for the inducement of anesthesia and/or sedation. In further embodiments, such compounds may behave as “soft drugs.”

Thus, in one aspect, the present invention provides a method of inducing sedation and/or anesthesia in a subject, comprising administering to the subject an effective amount of a compound of the present invention or a composition thereof. In certain embodiments, the compound is administered by intravenous administration. In certain embodiments, the compound is metabolized in vivo, e.g., by non-specific esterases, for example, peripheral esterases, to a less active or inactive compound. In certain embodiments, the method provides a faster recovery from sedation compared to other standard of care sedatives or anesthetics.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

An “effective amount” means the amount of a compound that, when administered to a subject is sufficient to induce anesthesia or sedation. The “effective amount” can vary depending on the compound, and the age, weight, etc., of the subject to be treated.

In yet another aspect, provided is a combination of a compound of the present invention and another pharmacologically active agent. The compounds provided herein can be administered as the sole active agent or they can be administered in combination with other agents. Administration in combination can proceed by any technique apparent to those of skill in the art including, for example, separate, sequential, concurrent and alternating administration.

In another aspect, provided is a method of treating or preventing brain excitability in a subject susceptible to or afflicted with a condition associated with brain excitability, comprising administering to the subject an effective amount of a compound of the present invention to the subject.

In yet another aspect, provided is a method of treating or preventing stress or anxiety in a subject, comprising administering to the subject in need of such treatment an effective amount of a compound of the present invention, or a composition thereof.

In yet another aspect, provided is a method of alleviating or preventing seizure activity in a subject, comprising administering to the subject in need of such treatment an effective amount of a compound of the present invention.

In yet another aspect, provided is a method of alleviating or preventing status epilepticus activity in a subject, comprising administering to the subject in need of such treatment an effective amount of a compound of the present invention.

In yet another aspect, provided is a method of alleviating or preventing insomnia in a subject, comprising administering to the subject in need of such treatment an effective amount of a compound of the present invention, or a composition thereof.

In yet another aspect, provided is a method of inducing sleep and maintaining substantially the level of REM sleep that is found in normal sleep, wherein substantial rebound insomnia is not induced, comprising administering an effective amount of a compound of the present invention.

In yet another aspect, provided is a method of alleviating or preventing PMS or PND in a subject, comprising administering to the subject in need of such treatment an effective amount of a compound of the present invention.

In yet another aspect, provided is a method of treating or preventing mood disorders in a subject, comprising administering to the subject in need of such treatment an effective amount of a compound of the present invention. In certain embodiments the mood disorder is depression.

In yet another aspect, provided is a method of treating or preventing traumatic brain injury in a subject, comprising administering to the subject in need of such treatment an effective amount of a compound of the present invention.

In yet another aspect, provided is a method of inducing anesthesia in a subject, comprising administering to the subject an effective amount of a compound of the present invention.

In yet another aspect, provided is a method of cognition enhancement or treating memory disorder by administering to the subject a therapeutically effective amount of a compound of the present invention. In certain embodiments, the disorder is Alzheimer's disease. In certain embodiments, the disorder is Rett syndrome.

In yet another aspect, provided is a method of treating attention disorders by administering to the subject a therapeutically effective amount of a compound of the present invention. In certain embodiments, the attention disorder is ADHD.

In certain embodiments, the compound is administered to the subject chronically.

In certain embodiments, the compound is administered to the subject intravenously. In certain embodiments, the compound is administered to the subject as an infusion. In certain embodiments, the compound is administered to the subject as a bolus infusion. In certain embodiments, the compound is administered to the subject as a continuous or sustained infusion.

### Administration of Neuroactive Steroid Formulations

A composition described herein, can be administered to a subject in need thereof, to treat a disorder, e.g., a central nervous system (CNS)-related disorder, e.g., a traumatic brain injury; e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; a seizure, e.g., acute repetitive seizures, cluster seizures. Although preferred patients are human, typically any mammal including domestic animals such as dogs, cats and horses, may also be treated.

Traumatic Brain Injury

The amount of the active ingredients to be administered is chosen based on the amount which provides the desired dose to the patient in need of such treatment to alleviate symptoms or treat a condition. Behavioral assays can be used to determine the rate and extent of behavior recovery in response to the treatment. Improved patient motor skills, spatial learning performance, cognitive function, sensory perception, speech and/or a decrease in the propensity to seizure may also be used to measure the neuroprotective effect. Such functional/behavioral tests used to assess sensorimortor and reflex function are described in, for example, Bederson et al. (1986) Stroke 17:472-476, DeRyck et al. (1992) Brain Res. 573:44-60, Markgraf et al. (1992) Brain Res. 575:238-246, Alexis et al. (1995) Stroke 26:2336-2346. Enhancement of neuronal survival may also be measured using the Scandinavian Stroke Scale (SSS) or the Barthl Index.

The treatment of a traumatic brain injury can be monitored by employing a variety of neurological measurements. For example, a partial therapeutic responses can be monitored by determining if, for example, there is an improvement in the subjects a) maximum daily Glasgow Coma Score; b) duration of coma; 3) daily intracranial pressure-therapeutic intensity levels; 4) extent of cerebral edema/mass effect measured on serial CT scans; and, 5) duration of ventilator support. A brief description of each of these assays is provided below.

The Glasgow Coma Score (index GCS) is a reflection of the depth of impaired consciousness and is best obtained following initial resuscitation (oxygenation, rehydration and support of blood pressure) but prior to use of sedating drugs, neuromuscular blocking agents, or endrotracheal intubation.

The duration of coma is defined as the number of hours from the time of injury that the subject is unable to purposefully respond to commands or mechanical stimulation. For non-intubated subjects, this equates to a GCS score of >8. For intubated patients, this correlates with a GCS motor score of .gtoreq.5. Duration of coma has been found to be predictive of functional outcome (Uhler et al. (1994) Neurosurgery 34(1): 122-8; Jiang et al. (1996) Brain Res 735(1): 101-7; and Gonzalez-Vidal et al. (1998) Arch Med Res 29(2): 117-24). Time spent in a coma induced pharmacologically for reasons other than brain injury should be subtracted in the final analysis.

The intracranial pressure (ICP) of patients with severe TBI is often monitored with an intracranial pressure device. Monitoring ICP can provide a measure of cerebral edema. However, inherent variability and analysis complexities due to therapeutic interventions intended on lowering the ICP mire using ICP measurements. To adjust for these interventions a therapeutic intensity scale was developed. This scale, known as the Therapeutic Intensity Level (TIL), measures treatment aggressiveness for elevated ICPs (Allolio et al. (1995) European Journal of Endocrinology 133(6): 696-700; Adashi et al. (1996) Reproductive endocrinology, surgery, and technology Philadelphia: Lippincott-Raven; and, Beers et al. eds. (1999) The Merck manual of diagnosis and therapy. 17th ed., Merck Sharp & Dohme Research Laboratories, Rahway, N.J.).

The extent of cerebral edema and mass effect can be determined by CT scans. For example, the volume of focal lesions can be measured. Mass lesions, either high-density or mixed-density abnormalities, will be evaluated by measuring the area of the abnormality as a region of interest, multiplying the area by the slice thickness, and summing these volumes for contiguous slices showing the same lesion. Each lesion will be measured three times, and the mean volume will be entered. This technique has been shown to be reliable (Garcia-Estrada et al. (1993) Brain Res 628(1-2): 271-8).

Intracerebral lesions can be further characterized by location (frontal, temporal, parietal, occipital, basal ganglia, or any combination). When an edematous zone is present, its volume (the hypodense perimeter) can be measured and analyzed separately. Midline shift will be measured using the septum pellucidum as the midline structure. The ventricle-brain ratio (VBR) will be calculated to quantify the degree of cerebral atrophy. Levin et al. ((1981) Archives of Neurology 38(10):623-9) found that the VBR had satisfactory reliability across different examiners, and was related both to the severity of acute injury and neurobehavioral sequelae (Hoffman et al. (1994) J Neurotrauma 11(4): 417-31).

The duration of ventilator support will be defined as the number of hours the patient receives positive pressure mechanical ventilation (Uhler et al. (1994) Veurosurgery 34(1): 122-8; Jiang et al. (1996) Brain Res 735(1): 101-7; and Gonzalez-Vidal et al. (1998) Arch Med Res 29(2): 117-24). Time spent under ventilator support for reasons other than brain injury will be subtracted in the final analysis.

In addition to the neurological measurements discussed above, a partial therapeutic response can also be assayed through various functional and neuropsychological outcomes. Several standardized measures of neuropsychological and functional performance are known. For instance subjects may display an improvement in the Glasgow Outcome Scale (GOS)/Glasgow Outcome Scale Extender (GOSE) and/or in the Disability Rating Scale (DRS). The Glasgow Outcome Score is one of the most widely used measures of brain injury recovery in the world (Garcia-Estrada et al. (1999) Int J Dev Neurosci 17(2): p. 145-51). Patients are classified into one of five categories: death, persistent vegetative state, severe disability, moderate disability, and good recovery. It is easy to administer and score, and has a high degree of reliability and validity.

The Disability Rating Scale (DRS) offers more precision than the GOS for measuring outcomes of moderate brain injury (Goodman et al. (1996) J Neurochem 66(5): 1836-44). The DRS consists of an eight-item rating of arousal and awareness, daily living activities, physical dependence, and employability (Vedder et al. (1999) J Neurochem 72(6):2531-8). Inter-rater reliability for the entire DRS is high (0.97 to 0.98).

The Functional Independence Measure (FIM) can be used to assess physical and cognitive disability. It contains 18 items in the following domains: self-care, sphincter control, mobility, locomotion, communication, and social cognition (Baulieu (1997) Mult Scler 3(2): 105-12). The FIM has demonstrated reliability and validity as an outcome measure following moderate and severe TBI (Jung-Testas et al. (1994) J Steroid Biochem Mol Biol 48(1): 145-54).

The Sickness Impact Profile is one method for measuring self-perceived health status (Schumacher et al. (1995) Ciba Found Symp 191: p. 90-112 and Koenig et al. (1995) Science 268(5216):1500-3). It consists of 136 questions divided into 12 categories: sleep and rest, eating, work, home management, recreation and pastimes, ambulation, mobility, body care and movement, social interaction, alertness, behavior, emotional behavior, and communication. It has been widely used across a variety of diseases and injuries, including head injury (Thomas et al. (1999) Spine 24:2134-8). Baseline SIP scores will reflect pre-injury health status, while follow-up scores will examine post-injury functioning.

Ischemia

Global ischemia, as used herein in reference to the CNS, refers to a condition which results from a general diminution of blood flow to the entire brain, forebrain, or spinal cord, which causes the delayed death of neurons, particularly those in metabolically active loci, throughout these tissues.

Focal ischemia, as used herein in reference to the CNS, refers to a condition that results from the blockage of a single artery that supplies blood to the brain or spinal cord, resulting in the death of all cellular elements (pan-necrosis) in the territory supplied by that artery.

Epilepsy

Epilepsy is a brain disorder characterized by repeated seizures overtime. Types of epilepsy can include, but are not limited to generalized epilepsy, e.g., childhood absence epilepsy, juvenile nyoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

Status Epilepticus (SE)

Status epilepticus (SE) caninclude, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

Compositions described herein can also be administered as a prophylactic to a subject having a CNS disorder e.g., a traumatic brain injury, status epilepticus, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges; prior to the onset of a seizure.

Seizures

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures.

The present invention will be further understood by reference to the following non-limiting examples.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

### EXAMPLES

In order that the invention described herein may be more fully understood, the following examples are set forth. The synthetic and biological examples described in this application are offered to illustrate the compounds, pharmaceutical compositions and methods provided herein and are not to be construed in any way as limiting their scope.

### Synthesis of 7

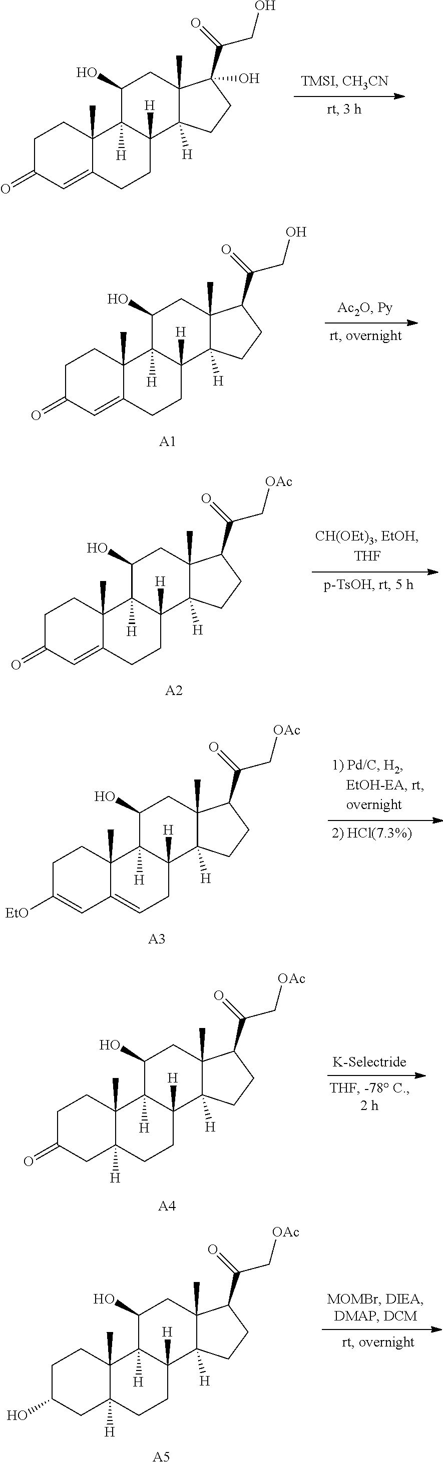

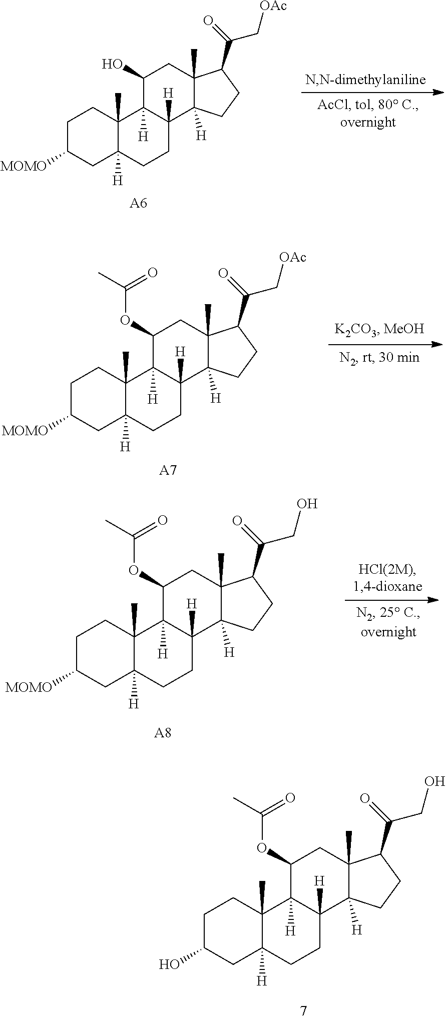

### 1. Synthesis of A1

Into a 5-L 4-necked round-bottom flask, was placed (8S,9S,10R,11S,13S,14S,17R)-11,17-dihydroxy-17-(2-hydroxyacetyl)-10,13-dimethyl-1,7,8,10,11,12,13,15,16,17-decahydro-2H-cyclopenta[a]phenanthren-3(6H,9H,14H)-one (15 g, 41.38 mmol, 1.00 equiv), CH₃CN (5 L), TMSI (29.6 mL). The resulting solution was stirred for 3 h at room temperature. The reaction was then quenched by the addition of 40 mL of Triethylamine. The resulting mixture was concentrated under vacuum. The resulting solution was extracted with 3×300 mL of dichloromethane and the organic layers combined. The resulting mixture was washed with 1×300 mL of sodium chloride. The mixture was dried over anhydrous sodium sulfate and concentrated under vacuum. The residue was applied onto a silica gel column with ethyl acetate/petroleum ether (55:45). This resulted in 6.7 g (45%) of A1, (8S,9S,10R,11S,13S,14S,17S)-11-hydroxy-17-(2-hydroxyacetyl)-10,13-dimethyl-1,7,8,10,11,12,13,15,16,17-decahydro-2H-cyclopenta[a]phenanthren-3(6H,9H,14H)-one as yellow oil.

¹H-NMR-A1: (300 MHz, CDCl₃): δ 5.696 (s, 1H), 4.432-4.387 (t, J=3 Hz, 1H), 4.146-4.200 (d, J=16.2 Hz, 2H), 1.195 (s, 3H), 0.947 (s, 3H).

### 2. Synthesis of A2

Into a 250-mL 3-necked round-bottom flask, was placed A1, (8S,9S,10R,11S,13S,14S,17S)-11-hydroxy-17-(2-hydroxyacetyl)-10,13-dimethyl-1,7,8,10,11,12,13,15,16,17-decahydro-2H-cyclopenta[a]phenanthren-3(6H,9H,14H)-one (9.9 g, 28.57 mmol, 1.00 equiv), Pyridine (110 mL), Ac₂O (4.4 g). The resulting solution was stirred for 1 overnight at room temperature. The resulting mixture was concentrated under vacuum. The resulting solution was extracted with 3×300 mL of ethyl acetate and the organic layers combined. The resulting mixture was washed with 1×200 mL of hydrogen chloride and 1×300 mL of sodium bicarbonate. The resulting mixture was washed with 1×300 mL of sodium chloride. The mixture was dried over anhydrous sodium sulfate and concentrated under vacuum. The residue was applied onto a silica gel column with ethyl acetate/petroleum ether (52:48). This resulted in 8.52 g (75%) of A2, 2-((8S,9S,10R,11S,13S,14S,17S)-11-hydroxy-10,13-dimethyl-3-oxo-2,3,6,7,8,9,10,11,12,13,14,15,16,17-tetradecahydro-1H-cyclopenta[a]phenanthren-17-yl)-2-oxoethyl acetate as yellow oil.

¹H-NMR-A2: (300 MHz, CDCl₃): δ 5.682 (s, 1H), 4.748-4.692 (d, J=16.5 Hz, 1H), 4.585-4.538 (d, J=14.1 Hz, 1H), (t, J=27.6 Hz, 2H), 4.108-4.408 (d, J=3 Hz, 1H), 2.175 (s, 3H), 1.444 (s, 3H), 0.948 (s, 3H).

### 3. Synthesis of A3

Into a 250-mL round-bottom flask, was placed A2, 2-((8S,9S,10R,11S,13S,14S,17S)-11-hydroxy-10,13-dimethyl-3-oxo-2,3,6,7,8,9,10,11,12,13,14,15,16,17-tetradecahydro-1H-cyclopenta[a]phenanthren-17-yl)-2-oxoethyl acetate (7.3 g, 18.79 mmol, 1.00 equiv), tetrahydrofuran (255.5 mL), ethanol (18.3 mL), p-TsOH (292 mg), CH(OEt)₃ (28.5 mL, 9.00 equiv). The resulting solution was stirred for 3 h at room temperature. The reaction was then quenched by the addition of 1 mL of Pyridine. The resulting solution was extracted with 3×80 mL of ethyl acetate and the organic layers combined. The resulting mixture was washed with 1×80 mL of sodium chloride. The mixture was dried over anhydrous sodium sulfate and concentrated under vacuum. The residue was applied onto a silica gel column with ethyl acetate/petroleum ether (18:82). This resulted in 1.88 g (18%) of A3, 2-((8S,9S,10R,11S,13S,14S,17S)-3-ethoxy-11-hydroxy-10,13-dimethyl-2,7,8,9,10,11,12,13,14,15,16,17-dodecahydro-1H-cyclopenta[a]phenanthren-17-yl)-2-oxoethyl acetate as yellow oil.

¹H-NMR-A3: (300 MHz, CDCl₃): δ 5.683 (s, 1H), 4.747-4.691 (d, J=16.8 Hz, 1H), 4.528-4.478 (d, J=15 Hz, 1H), 4.418-4.408 (d, J=3 Hz, 1H), 3.758-3.688 (m, 2H), 2.194 (s, H), 0.949 (s, 3H).

### 4. Synthesis of A4

Into a 100-mL round-bottom flask, was placed 2-((8S,9S,10R,11S,13S,14S,17S)-3-ethoxy-11-hydroxy-10,13-dimethyl-2,7,8,9,10,11,12,13,14,15,16,17-dodecahydro-1H-cyclopenta[a]phenanthren-17-yl)-2-oxoethyl acetate (2.73 g, 6.55 mmol, 1.00 equiv), ethanol/EtOAc (1:1) (54.6 mL), 10% Palladium carbon (273 mg). To the above hydrogen was introduced in. The resulting solution was stirred for 1 overnight at room temperature. The solids were filtered out. The resulting solution was extracted with 3×40 mL of ethyl acetate and the organic layers combined. The resulting mixture was washed with 1×40 mL of sodium chloride. The mixture was dried over anhydrous sodium sulfate and concentrated under vacuum. The residue was applied onto a silica gel column with ethyl acetate/petroleum ether (38:62). This resulted in 0.9 g (35%) of 2-((5S,8S,9S,10S,11S,13S,14S,17S)-11-hydroxy-10,13-dimethyl-3-oxo-hexadecahydro-1H-cyclopenta[a]phenanthren-17-yl)-2-oxoethyl acetate as a white solid.

¹H-NMR-A4: (300 MHz, CDCl₃): δ 4.736-4.680 (d, J=16.8 Hz, 1H), 4.546-4.490 (d, J=16.8 Hz, 1H), 4.386-4.375 (d, J=3.3 Hz, 1H), 2.187 (s, 3H), 1.262 (s, 3H), 0.913 (s, 3H).

### 5. Synthesis of A5

Into a 50-mL 3-necked round-bottom flask, was placed 2-((5S,8S,9S,10S,11S,13S,14S,17S)-11-hydroxy-10,13-dimethyl-3-oxo-hexadecahydro-1H-cyclopenta[a]phenanthren-17-yl)-2-oxoethyl acetate (1 g, 2.56 mmol, 1.00 equiv), tetrahydrofuran (20 mL), K-Selectride (2.8 mL, 1.10 equiv). The resulting solution was stirred for 1.5 h at −78° C. in a liquid nitrogen bath. The reaction was then quenched by the addition of 2 mL of H₂O₂. The resulting solution was extracted with 3×100 mL of ethyl acetate and the organic layers combined. The resulting mixture was washed with ×100 mL of brine. The mixture was dried over anhydrous sodium sulfate. The residue was applied onto a silica gel column with ethyl acetate/petroleum ether (1/1). This resulted in 500 mg (50%) of 2-((3R,5S,8S,9S,10S,11S,13S,14S,17S)-3,11-dihydroxy-10,13-dimethyl-hexadecahydro-1H-cyclopenta[a]phenanthren-17-yl)-2-oxoethyl acetate as a white solid.

¹H-NMR-A5: (400 NHz, CDCl₃): δ 4.740-4.698 (d, J=16.8 Hz, 1H), 4.583-4.541 (d, J=16.8 Hz, 1H), 4.418-4.410 (d, J=3.2 Hz, 1H), 4.084 (s, 1H), 2.166 (s, 3H), 1.045 (s, 3H), 0.902 (s, 3H).

### 6. Synthesis of A6

Into a 50-mL round-bottom flask, was placed 2-((3R,5S,8S,9S,10S,11S,13S,14S,17S)-3,11-dihydroxy-10,13-dimethyl-hexadecahydro-1H-cyclopenta[a]phenanthren-17-yl)-2-oxoethyl acetate (500 mg, 1.27 mmol, 1.00 equiv), DIEA (670 mg, 5.18 mmol, 4.00 equiv), 4-dimethylaminopyridine (28 mg, 0.23 mmol, 0.20 equiv), dichloromethane (10 mL). This was followed by the addition of MOMBr (364 mg, 2.00 equiv) dropwise with stirring at 0° C. The resulting solution was stirred overnight at room temperature. The resulting solution was extracted with 3×100 mL of dichloromethane and the organic layers combined. The resulting mixture was washed with 100 mL of brine. The mixture was dried over anhydrous sodium sulfate. The residue was applied onto a silica gel column with ethyl acetate/petroleum ether (1/1). This resulted in 300 mg (54%) of 2-((3R,5S,8S,9S,10S,11S,13S,14S,17S)-11-hydroxy-3-(methoxymethoxy)-10,13-dimethyl-hexadecahydro-1H-cyclopenta[a]phenanthren-17-yl)-2-oxoethyl acetate as a white solid.

### 7. Synthesis of A7

Into a 50-mL round-bottom flask, was placed 2-((3R,5S,8S,9S,10S,11S,13S,14S,17S)-11-hydroxy-3-(methoxymethoxy)-10,13-dimethyl-hexadecahydro-1H-cyclopenta[a]phenanthren-17-yl)-2-oxoethyl acetate (300 mg, 0.69 mmol, 1.00 equiv), N,N-dimethylaniline (7.2 mL), methylbenzene (10.8 g, 117.21 mmol, 170.58 equiv). This was followed by the addition of acetyl chloride (3.6 mL) dropwise with stirring at 0° C. The resulting solution was stirred overnight at 80° C. The resulting mixture was washed with ×200 mL of hydrogen chloride. The resulting solution was extracted with 3×100 mL of ethyl acetate and the organic layers combined. The resulting mixture was washed with 100 mL of brine. The mixture was dried over anhydrous sodium sulfate. The crude product (300 mg) was purified by Flash-Prep-HPLC with the following conditions (CombiFlash-1): Column, C18 silica gel; mobile phase, ACN:H₂O=2/1 increasing to ACN:H₂O=1:0; Detector, ELSD. 100 mg product was obtained. This resulted in 100 mg (30%) of A7 as yellow oil.

LC-MS-A7: (ES, m/z): 542[M+ACN+Na]⁺

### 8. Synthesis of A8

Into a 50-mL 3-necked round-bottom flask purged and maintained with an inert atmosphere of nitrogen, was placed A7 (100 mg, 0.21 mmol, 1.00 equiv), methanol (5 mL), a solution of potassium carbonate (700 mg, 5.06 mmol, 24.24 equiv) in water (5 mL). The resulting solution was stirred for 1 h at room temperature. The resulting solution was extracted with 3×100 mL of dichloromethane and the organic layers combined. The resulting mixture was washed with 100 mL of brine. The mixture was dried over anhydrous sodium sulfate and concentrated under vacuum. This resulted in 90 mg (crude) of (3R,5S,8S,9S,10S,11S,13S,14S,17S)-17-(2-hydroxyacetyl)-3-(methoxymethoxy)-10,13-dimethyl-hexadecahydro-1H-cyclopenta[a]phenanthren-11-yl acetate as a white solid.

### 9. Synthesis of 7

Into a 25-mL round-bottom flask, was placed (3R,5S,8S,9S,10S,11S,13S,14S,17S)-17-(2-hydroxyacetyl)-3-(methoxymethoxy)-10,13-dimethyl-hexadecahydro-1H-cyclopenta[a]phenanthren-11-yl acetate (260 mg, 0.62 mmol, 1.00 equiv), 1,4-dioxane (8 mL), hydrogen chloride (4 mL). The resulting solution was stirred overnight at room temperature. The resulting solution was extracted with 3×100 mL of ethyl acetate and the organic layers combined. The resulting mixture was washed with 100 mL of brine. The mixture was dried over anhydrous sodium sulfate. The crude product was purified by Flash-Prep-HPLC with the following conditions (IntelFlash-1): Column, C18 silica gel; mobile phase, ACN:H₂O=¼ increasing to ACN:H₂O=⅓; Detector, ELSD. This resulted in 123.2 mg (49%) of 7, (3R,5S,8S,9S,10S,11S,13S,14S,17S)-3-hydroxy-17-(2-hydroxyacetyl)-10,13-dimethyl-hexadecahydro-1H-cyclopenta[a]phenanthren-11-yl acetate as a white solid.

LC-MS-7: (ES, m/z): 391[M−1]

¹H-NMR-7: (400 MHz, CD₃OD): δ 5.396 (s, 1H), 4.152 (s, 2H), 3.968 (s, 1H), 2.618-2.565 (m, 1H), 2.004 (s, 3H), 0.932 (s, 3H), 0.779 (s, 3H).

### Synthesis of 6

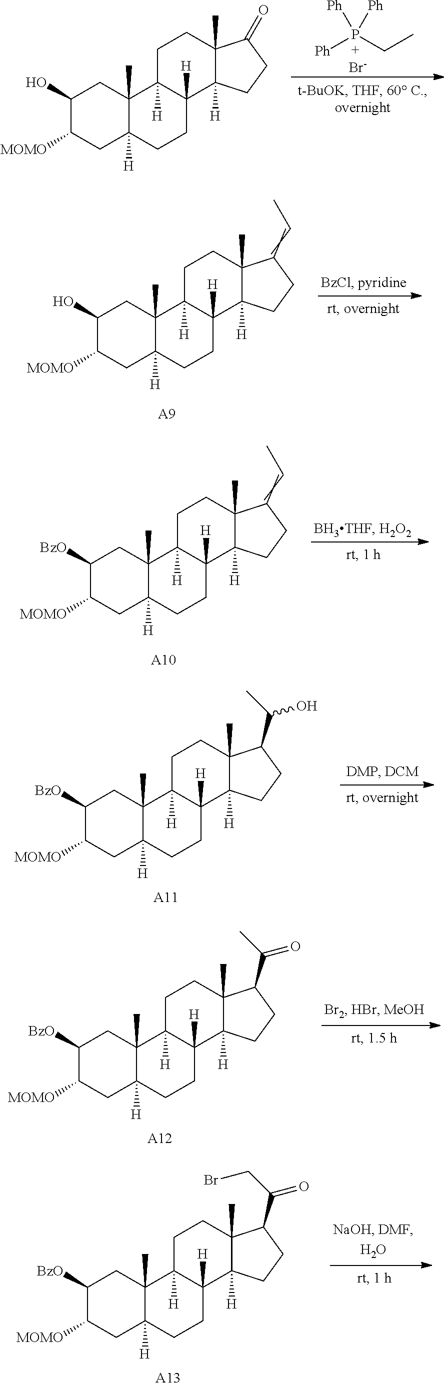

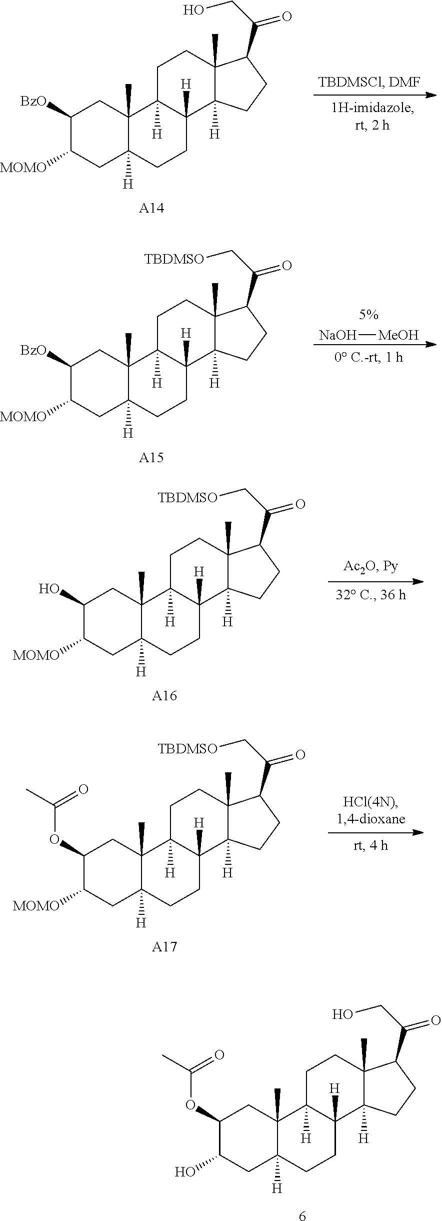

### 1. Synthesis of A9

Into a 1000-mL 3-necked round-bottom flask purged and maintained with an inert atmosphere of nitrogen, was placed ethyltriphenylphosphanium bromide (37.1 g, 99.93 mmol, 5.00 equiv), tetrahydrofuran (300 mL), potassium t-butoxide (154 mL, 7.70 equiv). The resulting solution was stirred for 90 min at 60° C. in an oil bath. Then (2S,3S,5S,8R,9S,10S,13S,14S)-2-hydroxy-3-(methoxymethoxy)-10,13-dimethyl-tetradecahydro-2H-cyclopenta[a]phenanthren-17(14H)-one (7 g, 19.97 mmol, 1.00 equiv) in THF (20 mL) was added. The resulting solution was allowed to react, with stirring, for an additional 1 overnight while the temperature was maintained at 60° C. in an oil bath. The reaction was then quenched by the addition of 200 mL of water. The resulting solution was extracted with 3×200 mL of ethyl acetate, the organic layers combined and washed with brine (200 mL×1), dried over anhydrous sodium sulfate and concentrated under vacuum. The residue was applied onto a silica gel column with ethyl acetate/petroleum ether (1:4). This resulted in 8.5 g (crude) of (1S,2S,4S,5(2S,3S,5S,8R,9S,10S,13S,14S)-17-ethylidene-3-(methoxymethoxy)-10,13-dimethyl-hexadecahydro-1H-cyclopenta[a]phenanthren-2-ol as a white solid.

¹H-NMR-A9: (300 Hz, CDCl₃): δ 5.099-5.123 (d, J=7.2 Hz, 1H), 4.643-4.703 (m, 2H), 3.979-3.989 d, J=3 Hz, 1H), 3.676-3.685 (d, J=2.7 Hz, 1H), 3.388 (s, 3H), 1.002 (s, 3H), 0.865 (s, 3H)

### 2. Synthesis of A10

Into a 250-mL 3-necked round-bottom flask, was placed (2S,3S,5S,8R,9S,10S,13S,14S)-17-ethylidene-3-(methoxymethoxy)-10,13-dimethyl-hexadecahydro-1H-cyclopenta[a]phenanthren-2-ol (8.5 g, 23.45 mmol, 1.00 equiv), pyridine (85 mL). This was followed by the addition of BzCl (5.9 g, 41.97 mmol, 1.80 equiv) dropwise with stirring at 0° C. The resulting solution was stirred for 1 overnight at 25° C. The reaction was then quenched by the addition of 50 mL of water. The resulting mixture was concentrated under vacuum. The resulting solution was diluted with 150 mL of H₂O. The resulting solution was extracted with 3×150 mL of ethyl acetate and the organic layers combined, washed with brine (200×1 mL), dried over anhydrous sodium sulfate and concentrated under vacuum. The residue was applied onto a silica gel column with ethyl acetate/petroleum ether (1:9). This resulted in 12.9 g (crude) of (2S,3S,5S,8R,9S,10S,13S,14S)-17-ethylidene-3-(methoxymethoxy)-10,13-dimethyl-hexadecahydro-1H-cyclopenta[a]phenanthren-2-yl benzoate as white oil.

¹H-NMR-A10: (300 Hz, CDCl₃): δ 8.105-8.134 (t, J=8.7 Hz, 1H), 8.020-8.048 (t, J=8.4 Hz, 1H), 7.422-7.620 (m, 3H), 5.251-5.259 (d, J=2.4 Hz, 1H), 4.707-4.782 (m, 2H), 3.850-3.857 (d, J=1.8 Hz, 1H), 3.414 (s, 3H),1.045 (s, 3H), 0.850 (s, 3H)

### 3. Synthesis of A11

Into a 1000-mL 3-necked round-bottom flask, was placed (2S,3S,5S,8R,9S,10S,13S,14S)-17-ethylidene-3-(methoxymethoxy)-10,13-dimethyl-hexadecahydro-1H-cyclopenta[a]phenanthren-2-yl benzoate (12.9 g, 27.64 mmol, 1.00 equiv), tetrahydrofuran (130 mL). This was followed by the addition of BH₃(1M in THF) (49.7 mL, 1.80 equiv) dropwise with stirring at 0° C. The resulting solution was stirred for 1 h at 25° C. To this was added sodium hydroxide (160 mL, 10%) in several batches at 0° C. To the mixture was added H₂O2 (120 mL, 30%) in several batches at 0° C. The resulting solution was allowed to react, with stirring, for an additional 1 h at 25° C. The resulting solution was extracted with 3×200 mL of ethyl acetate and the organic layers combined. The resulting mixture was washed with 1×200 mL of 10% Na₂S₂O₃(aq) and 1×200 mL of brine. The solid was dried over anhydrous sodium sulfate and concentrated. This resulted in 12.9 g (96%) of (2S,3S,5S,8R,9S,10S,13S,14S,17S)-17-(1-hydroxyethyl)-3-(methoxymethoxy)-10,13-dimethyl-hexadecahydro-1H-cyclopenta[a]phenanthren-2-yl benzoate as colorless oil.

¹H-NMR-A11: (300 Hz, CDCl₃): δ 8.011-8.016 (d, J=1.5 Hz, 2H), 7.418-7.565 (m, 3H), 5.247-5.254 (t, J=0.9 Hz, 1H), 4.700-4.785 (m, 3H), 3.847-3.854 (d, J=2.4 Hz, 1H), 3.610-3.798 (m, 1H), 3.401-3.417 (t, J=2.4 Hz, 3H), 1.032 (s, 3H), 0.634 (s, 3H)

### 4. Synthesis of A12

Into a 500-mL round-bottom flask, was placed (2S,3S,5S,8R,9S,10S,13S,14S,17S)-17-(1-hydroxyethyl)-3-(methoxymethoxy)-10,13-dimethyl-hexadecahydro-1H-cyclopenta[a]phenanthren-2-yl benzoate (12.9 g, 26.62 mmol, 1.00 equiv), dichloromethane (130 mL). This was followed by the addition of DMP (20.2 g, 47.63 mmol, 1.80 equiv) in several batches at 0° C. The resulting solution was stirred for 1 overnight at 25° C. The solids were filtered out and the filter cake was washed with DCM (100 mL×3) The filterate was concentrated under vacuum. The residue was applied onto a silica gel column with ethyl acetate/petroleum ether (1:9). This resulted in 14.5 g (crude) of (2S,3S,5S,8R,9S,10S,13S,14S,17S)-17-acetyl-3-(methoxymethoxy)-10,13-dimethyl-hexadecahydro-1H-cyclopenta[a]phenanthren-2-yl benzoate as colorless oil.

¹H-NMR-A12: (400 Hz, CDCl₃): δ 8.024-8.090 (m, 2H), 7.568-7.587 (m, 1H), 7.447-7.486 (m, 2H), 5.274-5.279 (d, J=2 Hz, 1H), 4.739-4.805 (m, 2H), 3.878-3.884 (d, J=2.4 Hz, 1H), 3.431-3.442 (d, J=4.4 Hz, 3H), 2.552 (m, 1H), 2.147 (s, 3H), 1.052 (s, 3H), 0.607 (s, 3H)

### 5. Synthesis of A13

Into a 250-mL round-bottom flask, was placed (2S,3S,5S,8R,9S,10S,13S,14S,17S)-17-acetyl-3-(methoxymethoxy)-10,13-dimethyl-hexadecahydro-1H-cyclopenta[a]phenanthren-2-yl benzoate (14.5 g, 30.04 mmol, 1.00 equiv), methanol (150 mL). This was followed by the addition of a solution of 48% HBr(aq) (1.9 mL) in methanol (3.8 mL) dropwise with stirring at 0° C. To this was added Br₂ (5.3 g, 33.16 mmol, 1.00 equiv) dropwise with stirring at 0° C. The resulting solution was stirred for 1.5 h at 25° C. The reaction was then quenched by the addition of 27 g of CH₃COONa (27 g in 63 mL H₂O). The resulting solution was diluted with 200 mL of H₂O. The resulting solution was extracted with 3×200 mL of dichloromethane and the organic layers combined and dried over anhydrous sodium sulfate and concentrated under vacuum. The residue was applied onto a silica gel column with ethyl acetate/petroleum ether (8:100). This resulted in 10.5 g (62%) of (2S,3S,5S,8R,9S,10S,13S,14S,17S)-17-(2-bromoacetyl)-3-(methoxymethoxy)-10,13-dimethyl-hexadecahydro-1H-cyclopenta[a]phenanthren-2-yl benzoate as a white foam.

¹H-NMR-A13: (400 Hz, CDCl₃): δ 8.032-8.035 (m, 1H), 7.567-7.585 (m, 1H), 7.446-7.484 (m, 2H), 5.268-5.274 (d, J=2.4 Hz, 1H), 4.715-4.797 (m, 2H), 3.873-3.927 (m, 3H), 3.423-3.434 (d, J=3.6 Hz, 3H), 2.837 (m, 1H), 1.023-1.050 (d, J=10.8 Hz, 3H), 0.631 (s, 3H)

### 6. Synthesis of A14

Into a 250-mL round-bottom flask, was placed (2S,3S,5S,8R,9S,10S,13S,14S,17S)-17-(2-bromoacetyl)-3-(methoxymethoxy)-10,13-dimethyl-hexadecahydro-1H-cyclopenta[a]phenanthren-2-yl benzoate (10.5 g, 18.70 mmol, 1.00 equiv), N,N-dimethylformamide (110 mL). This was followed by the addition of a solution of sodium hydroxide (900 mg, 22.50 mmol, 1.20 equiv) in water (10 mL) dropwise with stirring at 0° C. The resulting solution was stirred for 60 min at 25° C. The reaction was then quenched by the addition of 100 mL of saturated NH₄Cl(aq). The resulting solution was diluted with 100 mL of H₂O. The resulting solution was extracted with 3×200 mL of ethyl acetate and the organic layers combined and dried over anhydrous sodium sulfate and concentrated under vacuum. The residue was applied onto a silica gel column with ethyl acetate/petroleum ether (1:5). This resulted in 3.3 g (35%) of (2S,3S,5S,8R,9S,10S,13S,14S,17S)-17-(2-hydroxyacetyl)-3-(methoxymethoxy)-10,13-dimethyl-hexadecahydro-1H-cyclopenta[a]phenanthren-2-yl benzoate as a white foam.

¹H-NMR-A14: (300 Hz, CDCl₃): δ 8.008-8.012 (m, 2H), 7.420-7.567 (m, 3H), 5.247 (s, 1H), 4.695-4.781 (m, 2H), 4.164-4.183 (m, 2H), 3.854-3.860 (d, J=1.8 Hz, 1H), 3.404 (s, 3H), 1.032 (s, 3H), 0.640 (s, 3H)

### 7. Synthesis of A15

Into a 50-mL round-bottom flask, was placed (2S,3S,5S,8R,9S,10S,13S,14S,17S)-17-(2-hydroxyacetyl)-3-(methoxymethoxy)-10,13-dimethyl-hexadecahydro-1H-cyclopenta[a]phenanthren-2-yl benzoate (2.5 g, 5.01 mmol, 1.00 equiv), N,N-dimethylformamide (7.5 mL), pyridine (5 mL), imidazole (1.01 g, 3.00 equiv). This was followed by the addition of TBDMSCl (2.27 g, 3.00 equiv) in several batches at 0° C. The resulting solution was stirred for 2 h at 25° C. The reaction was then quenched by the addition of 100 mL of water. The resulting solution was extracted with 3×100 mL of ethyl acetate and the organic layers combined. The resulting mixture was washed with 1×200 mL of Brine. The mixture was dried over anhydrous sodium sulfate and concentrated under vacuum. The residue was applied onto a silica gel column with ethyl acetate/petroleum ether (1:10). This resulted in 2.0 g (65%) of (2S,3S,5S,8R,9S,10S,13S,14S,17S)-17-(2-(tert-butyldimethylsilyloxy)acetyl)-3-(methoxymethoxy)-10,13-dimethyl-hexadecahydro-1H-cyclopenta[a]phenanthren-2-yl benzoate as colorless oil.

¹H-NMR-A15: (400 Hz, CDCl₃): δ 8.038-8.056 (d, J=7.2 Hz, 2H), 7.448-7.587 (m, 3H), 5.268 (s, 1H), 4.730-4.797 (m, 2H), 4.136-4.213 (m, 2H), 3.876-3.880 (d, J=1.2 Hz, 1H), 3.425-3.436 (t, J=0.9 Hz, 3H), 1.025 (s, 3H), 0.936 (s, 9H), 0.661 (s, 3H), 0.108 (s, 6H)

### 8. Synthesis of A16

Into a 25-mL round-bottom flask purged and maintained with an inert atmosphere of nitrogen, was placed (2S,3S,5S,8R,9S,10S,13S,14S,17S)-17-(2-(tert-butyldimethylsilyloxy)acetyl)-3-(methoxymethoxy)-10,13-dimethyl-hexadecahydro-1H-cyclopenta[a]phenanthren-2-yl benzoate (1.0 g, 1.63 mmol, 1.00 equiv), tetrahydrofuran (5.0 mL). This was followed by the addition of a solution of sodium hydroxide (5.0 g, 12.50 mmol, 7.66 equiv) in methanol (5.0 mL) dropwise with stirring at 0° C. The resulting solution was stirred for 1 h at 25° C. The reaction was then quenched by the addition of 50 mL of saturated NH₄Cl(aq). The resulting solution was extracted with 3×50 mL of ethyl acetate and the organic layers combined. The resulting mixture was washed with 1×100 mL of Brine. The mixture was dried over anhydrous sodium sulfate. The residue was applied onto a silica gel column with ethyl acetate/petroleum ether (1:4). This resulted in 400 mg (48%) of 2-(tert-butyldimethylsilyloxy)-1-((2S,3S,5S,8R,9S,10S,13S,14S,17S)-2-hydroxy-3-(methoxymethoxy)-10,13-dimethyl-hexadecahydro-1H-cyclopenta[a]phenanthren-17-yl)ethanone as a white foam.

¹H-NMR-A16: (300 Hz, CDCl₃): δ 4.642-4.702 (m, 2H), 4.167-4.194 (m, 2H), 3.980 (s, 1H), 3.674 (s, 1H), 3.372 (s, 3H), 0.011 (s, 6H)

### 9. Synthesis of A17

Into a 8-mL vial, was placed 2-(tert-butyldimethylsilyloxy)-1-((2S,3S,5S,8R,9S,10S,13S,14S,17S)-2-hydroxy-3-(methoxymethoxy)-10,13-dimethyl-hexadecahydro-1H-cyclopenta[a]phenanthren-17-yl)ethanone (250 mg, 0.49 mmol, 1.00 equiv), pyridine (3 mL). This was followed by the addition of acetic anhydride (120 mg, 2.00 equiv) dropwise with stirring at 0° C. The resulting solution was stirred for 36 h at 32° C. in an oil bath. The reaction was then quenched by the addition of 20 mL of water. The resulting mixture was concentrated under vacuum. The resulting solution was diluted with 20 mL of H₂O. The resulting solution was extracted with 3×20 mL of ethyl acetate and the organic layers combined and dried over anhydrous sodium sulfate. The residue was applied onto a silica gel column with ethyl acetate/petroleum ether (15:100). This resulted in 250 mg (92%) of (2S,3S,5S,8R,9S,10S,13S,14S,17S)-17-(2-(tert-butyldimethylsilyloxy)acetyl)-3-(methoxymethoxy)-10,13-dimethyl-hexadecahydro-1H-cyclopenta[a]phenanthren-2-yl acetate as colorless oil.

### 10. Synthesis of 6

Into a 8-mL vial, was placed 2-[(tert-butyldimethylsilyl)oxy]-1-[(1S,2S,4S,5S,7S,10R,11S,14S,15S)-4-hydroxy-5-(methoxymethoxy)-2,15-dimethyltetracyclo[8.7.0.0̂[2,7].0̂[11,15]]heptadecan-14-yl]ethan-1-one (50 mg, 0.10 mmol, 1.00 equiv), dioxane (0.5 mL). This was followed by the addition of hydrogen chloride aqueous (0.5 mL) dropwise with stirring at 0° C. The resulting solution was stirred for 1 h at 25° C. The reaction was then quenched by the addition of 20 mL of water. The resulting solution was extracted with 3×20 mL of ethyl acetate and the organic layers combined. The resulting mixture was washed with 1×50 mL of brine. The mixture was dried over anhydrous sodium sulfate and concentrated under vacuum. The crude product (100 mg) was purified by Prep-HPLC with the following conditions (1#-Waters-2676): Column, X-bridge Prep phenyl 5 um, 19*150 mmh Prep C012(T)186003581138241113.01; mobile phase, water in 0.05% NH₄HCO₃ and CH₃CN (35% CH₃CN up to 45% in 13 min, up to 95% in 1 min, down to 35% in 1 min); Detector, ELSD. This resulted in 24.1 mg (70%) of 1-[(1S,2S,4S,5S,7S,10R,11S,14S,15S)-4,5-dihydroxy-2,15-dimethyltetracyclo[8.7.0.0̂[2,7].0̂[11,15]]heptadecan-14-yl]-2-hydroxyethan-1-one as a white solid.

LC-MS-6: (ES, m/z): 391[M−H]⁻

¹H-NMR-A17: (400 Hz, CDCl₃): δ 4.904 (s, 1H), 4.138-4.257 (m, 2H), 3.893 (s, 1H), 3.265 (br., 1H), 2.064 (s, 3H), 0.932 (s, 3H), 0.644 (s, 3H)

### Synthesis of 2

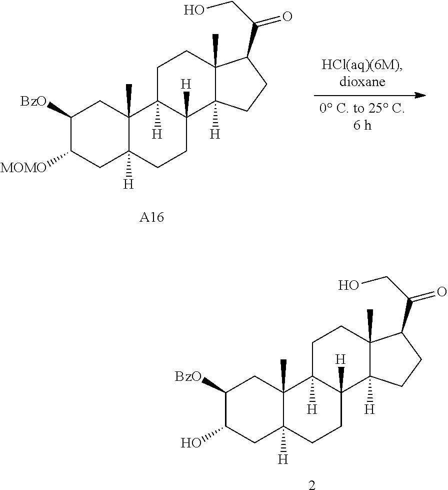

Into a 50-mL round-bottom flask, was placed (2S,3S,5S,8R,9S,10S,13S,14S,17S)-17-(2-hydroxyacetyl)-3-(methoxymethoxy)-10,13-dimethyl-hexadecahydro-1H-cyclopenta[a]phenanthren-2-yl benzoate (770 mg, 1.54 mmol, 1.00 equiv), dioxane (6 mL), hydrogen chloride (6M, 1 mL). The resulting solution was stirred for 6 h at 25° C. The resulting mixture was concentrated under vacuum. The resulting solution was diluted with 20 mL of H₂O. The resulting solution was extracted with 3×50 mL of ethyl acetate and the organic layers combined and dried over anhydrous sodium sulfate and concentrated. This resulted in 330 mg (47%) of (2S,3S,5S,8R,9S,10S,13S,14S,17S)-3-hydroxy-17-(2-hydroxyacetyl)-10,13-dimethyl-hexadecahydro-1H-cyclopenta[a]phenanthren-2-yl benzoate as a white foam.

LC-MS-2: (ES, m/z): 477[M+Na]⁺

¹H-NMR-2: (300 Hz, CDCl₃): δ 8.018 (m, 2H), 7.426-7.598 (m, 3H), 5.160 (s, 1H), 4.103-4.248 (m, 2H), 4.022 (s, 1H), 3.250 (br., 1H), 2.429-2.488 (m, 1H), 1.043 (s, 3H), 0.615 (s, 3H)

### Synthesis of 8 and 9

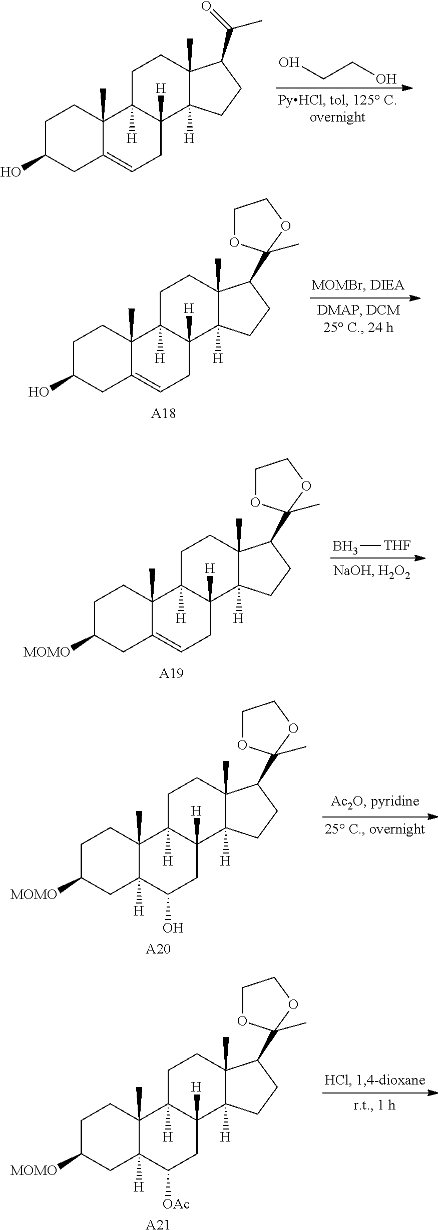

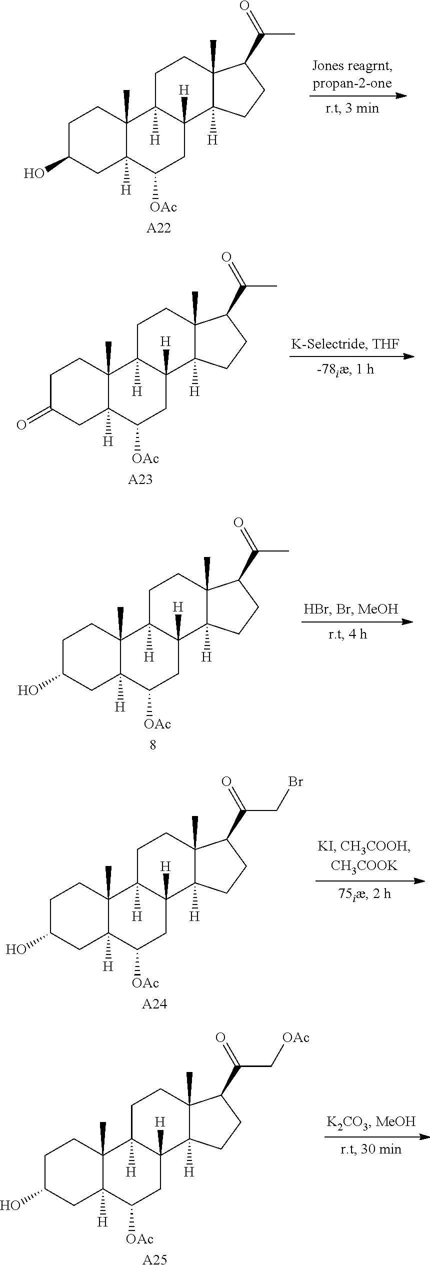

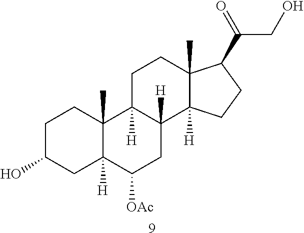

### 1. Synthesis of A18

Into a 1000-mL round-bottom flask, was placed 1-((3S,8S,9S,10R,13S,14S,17S)-3-hydroxy-10,13-dimethyl-2,3,4,7,8,9,10,11,12,13,14,15,16,17-tetradecahydro-1H-cyclopenta[a]phenanthren-17-yl)ethanone (50 g, 157.99 mmol, 1.00 equiv), tol (1200 mL), ethane-1,2-diol (392 g, 6.32 mol, 40.00 equiv), pyridine hydrochloride (1.84 g, 15.92 mmol, 0.10 equiv). The resulting solution was stirred for overnight at 125° C. in an oil bath. The resulting mixture was concentrated under vacuum. The solids were collected by filtration. Wash the filter cake with H₂O (200 mL*3) and EA (200 mL*2) This resulted in 49.2 g (86%) of (3S,8S,9S,10R,13S,14S,17S)-10,13-dimethyl-17-(2-methyl-1,3-dioxolan-2-yl)-2,3,4,7,8,9,10,11,12,13,14,15,16,17-tetradecahydro-1H-cyclopenta[a]phenanthren-3-ol as a white solid.

¹H-NMR-A18: (CDCl₃, 400 MHz): δ 5.357-5.370 (t, J=5.2 Hz, 1H), 3.848-4.011 (m, 4H), 3.325-3.333 (m, 1H), 1.291 (s, 3H), 1.061 (s, 3H), 0.816 (s, 3H).

### 2. Synthesis of A19

Into a 1000-mL round-bottom flask, was placed (3S,8S,9S,10R,13S,14S,17S)-10,13-dimethyl-17-(2-methyl-1,3-dioxolan-2-yl)-2,3,4,7,8,9,10,11,12,13,14,15,16,17-tetradecahydro-1H-cyclopenta[a]phenanthren-3-ol (49.2 g, 136.47 mmol, 1.00 equiv), tetrahydrofuran (60 g, 832.06 mmol, 6.10 equiv), DIEA (90 mL, 4.00 equiv), 4-dimethylaminopyridine (3.3 g, 27.01 mmol, 0.20 equiv). This was followed by the addition of MOMBr (28 mL, 2.50 equiv) dropwise with stirring at 0° C. The resulting solution was stirred for 1 overnight at 25° C. The reaction was then quenched by the addition of 400 mL of water. The resulting solution was extracted with 2×300 mL of ethyl acetate and the organic layers combined and dried over anhydrous sodium sulfate and concentrated under vacuum. The residue was applied onto a silica gel column with ethyl acetate/petroleum ether (10:90). This resulted in 50 g (91%) of 2-((3S,8S,9S,10R,13S,14S,17S)-3-(methoxymethoxy)-10,13-dimethyl-2,3,4,7,8,9,10,11,12,13,14,15,16,17-tetradecahydro-1H-cyclopenta[a]phenanthren-17-yl)-2-methyl-1,3-dioxolane as a white solid.

¹H-NMR-A19: (CDCl₃, 300 MHz): δ 5.346-5.363 (t, J=6.8 Hz, 1H), 4.681 (s, 2H), 3.883 (s, 3H), 3.718-3.739 (m, 1H), 3.370-3.397 (m, 4H), 1.300 (s, 3H), 1.014 (s, 3H), 0.778 (s, 3H).

### 3. Synthesis of A20

Into a 500-mL 3-necked round-bottom flask purged and maintained with an inert atmosphere of nitrogen, was placed 2-((3S,8S,9S,10R,13S,14S,17S)-3-(methoxymethoxy)-10,13-dimethyl-2,3,4,7,8,9,10,11,12,13,14,15,16,17-tetradecahydro-1H-cyclopenta[a]phenanthren-17-yl)-2-methyl-1,3-dioxolane (10 g, 24.72 mmol, 1.00 equiv), oxolane (100 mL). This was followed by the addition of oxolaneborane (43 mL, 2.00 equiv, 1M) dropwise with stirring at 0° C. The resulting solution was stirred for 1 overnight at 25° C. Then 146 mL 10% sodium hydroxide(aq) and 96 mL of 30% H₂O₂ (aq) was added. The resulting solution was stirred for 2 h at 25° C. The resulting solution was extracted with 2×200 mL of ethyl acetate and the organic layers combined. The resulting mixture was washed with 1×200 mL of 10% Na₂S₂O₃(aq) and 1×200 mL of brine. The mixture was dried over anhydrous sodium sulfate. The residue was applied onto a silica gel column with ethyl acetate/petroleum ether (22:100). This resulted in 6 g (57%) of (3S,5S,6S,8R,9S,10R,13S,14S,17S)-3-(methoxymethoxy)-10,13-dimethyl-17-(2-methyl-1,3-dioxolan-2-yl)-hexadecahydro-1H-cyclopenta[a]phenanthren-6-ol as a white solid.

¹H-NMR-A20: (CDCl₃, 300 MHz): δ 4.698 (s, 2H), 3.874-3.895 (m, 4H), 3.740-3.864 (m, 2H), 3.369 (s, 3H), 1.259 (s, 3H), 0.894 (s, 3H), 0.751 (s, 3H).

### 4. Synthesis of A21

Into a 500-mL round-bottom flask, was placed (3S,5S,6S,8R,9S,10R,13S,14S,17S)-3-(methoxymethoxy)-10,13-dimethyl-17-(2-methyl-1,3-dioxolan-2-yl)-hexadecahydro-1H-cyclopenta[a]phenanthren-6-ol (15 g, 35.49 mmol, 1.00 equiv), pyridine (150 mL). This was followed by the addition of acetic anhydride (7.23 g, 2.00 equiv) dropwise with stirring at 0° C. The resulting solution was stirred for 48 h at 35° C. in an oil bath. The reaction was then quenched by the addition of 100 mL of water. The resulting mixture was concentrated under vacuum. The resulting solution was diluted with 200 mL of H₂O. The resulting solution was extracted with 3×200 mL of ethyl acetate and the organic layers combined. The resulting mixture was washed with 1×300 mL of Brine. The mixture was dried over anhydrous sodium sulfate. The residue was applied onto a silica gel column with ethyl acetate/petroleum ether (20:80). This resulted in 16 g (97%) of (3S,5S,6S,8R,9S,10R,13S,14S,17S)-3-(methoxymethoxy)-10,13-dimethyl-17-(2-methyl-1,3-dioxolan-2-yl)-hexadecahydro-1H-cyclopenta[a]phenanthren-6-yl acetate as colorless oil.

¹H-NMR-A21: (CDCl₃, 300 MHz): δ4.695 (s, 2H), 3.322 (s, 3H), 2.050 (s, 3H), 1.268 (s, 3H), 0.954 (s, 3H), 0.790 (s, 3H).

### 5. Synthesis of A22

Into a 250-mL round-bottom flask, was placed (3S,5S,6S,8R,9S,10R,13S,14S,17S)-3-(methoxymethoxy)-10,13-dimethyl-17-(2-methyl-1,3-dioxolan-2-yl)-hexadecahydro-1H-cyclopenta[a]phenanthren-6-yl acetate (2.8 g, 6.03 mmol, 1.00 equiv), dioxane (67.2 mL), 2 mol/L HCl(aq) (33.6 mL). The resulting solution was stirred for 1 h at room temperature. The resulting solution was extracted with 3×30 mL of ethyl acetate and the organic layers combined. The resulting mixture was washed with 1×50 mL of sodium chloride. The mixture was dried over anhydrous sodium sulfate and concentrated under vacuum. The residue was applied onto a silica gel column with ethyl acetate/petroleum ether (32:68). This resulted in 1.4 g (62%) of (3S,5S,6S,8R,9S,10R,13S,14S,17S)-17-acetyl-3-hydroxy-10,13-dimethyl-hexadecahydro-1H-cyclopenta[a]phenanthren-6-yl acetate as yellow oil.

¹H-NMR-A22: (CDCl₃, 300 MHz): δ 4.669-4.684 (m, 1H), 3.557 (m, 1H), 2.111 (s, 3H), 2.044 (s, 3H), 0.748 (s, 3H), 0.608 (s, 3H)

### 6. Synthesis of A23

Into a 500-mL 3-necked round-bottom flask, was placed (3S,5S,6S,8R,9S,10R,13S,14S,17S)-17-acetyl-3-hydroxy-10,13-dimethyl-hexadecahydro-1H-cyclopenta[a]phenanthren-6-yl acetate (4.2 g, 11.15 mmol, 1.00 equiv), propan-2-one (168 mL), added to Jones reagent (4.2 mL) with dropwise. The resulting solution was stirred for 3 min at room temperature. The reaction was then quenched by the addition of 4 mL of isopropanol. The resulting mixture was concentrated under vacuum. The resulting solution was extracted with 3×40 mL of ethyl acetate and the organic layers combined. The resulting mixture was washed with 1×80 mL of sodium chloride. The mixture was dried over anhydrous sodium sulfate and concentrated under vacuum. The residue was applied onto a silica gel column with ethyl acetate/petroleum ether (18:82). This resulted in 3.8 g (91%) of (5S,6S,8R,9S,10R,13S,14S,17S)-17-acetyl-10,13-dimethyl-3-oxo-hexadecahydro-1H-cyclopenta[a]phenanthren-6-yl acetate as yellow oil.

¹H-NMR-A23: (CDCl₃, 300 MHz): δ4.715-4.773 (m, 1H), 2.120 (s, 3H), 2.077 (s, 3H), 1.043 (s, 3H), 0.642 (s, 3H).

### 7. Synthesis of 8

Into a 50-mL 3-necked round-bottom flask purged and maintained with an inert atmosphere of nitrogen, was placed (5S,6S,8R,9S,10R,13S,14S,17S)-17-acetyl-10,13-dimethyl-3-oxo-hexadecahydro-1H-cyclopenta[a]phenanthren-6-yl acetate (360 mg, 0.96 mmol, 1.00 equiv), tetrahydrofuran (10 mL). This was followed by the addition of K-Selectride (1.1 mL, 1.10 equiv) dropwise with stirring at −78° C. The resulting solution was stirred for 1 h at −78° C. The reaction was then quenched by the addition of 2 mL of H₂O₂ at −78° C. The resulting solution was extracted with 3×10 mL of ethyl acetate and the organic layers combined. The resulting mixture was washed with 1×10 mL of sodium chloride. The mixture was dried over anhydrous sodium sulfate and concentrated under vacuum. The residue was applied onto a silica gel column with ethyl acetate/petroleum ether (20:80). The crude product (250 mg) was purified by Flash-Prep-HPLC with the following conditions (IntelFlash-1): Column, SunFire Prep C18; mobile phase, CH₃CN/H₂O (0.05% TFA)=1:4 increasing to CH₃CN/H₂O (0.05% TFA)=4:1 within 20 min; Detector, ELSD. 121 mg product was obtained. This resulted in 121 mg (33%) of (3R,5S,6S,8R,9S,10R,13S,14S,17S)-17-acetyl-3-hydroxy-10,13-dimethyl-hexadecahydro-1H-cyclopenta[a]phenanthren-6-yl acetate as white solid.

LC-MS-8: (ES, m/z): 375[M−H]⁻

¹H-NMR-8: (CD₃OD, 400 MHz): δ 4.681-4.877 (m, 1H), 4.013-4.026 (m, 1H), 2.164 (s, 3H), 2.052 (s, 3H), 0.904 (s, 3H), 0.639 (s, 3H).

### 8. Synthesis of A24

Into a 250-mL round-bottom flask, was placed (3R,5S,6S,8R,9S,10R,13S,14S,17S)-17-acetyl-3-hydroxy-10,13-dimethyl-hexadecahydro-1H-cyclopenta[a]phenanthren-6-yl acetate (3 g, 7.97 mmol, 1.00 equiv), methanol (100 mL), a solution of 48% HBr (0.48 mL) in methanol (0.96 mL), Br₂ (0.44 mL). The resulting solution was stirred for 4 h at room temperature. The resulting mixture was concentrated under vacuum. The resulting solution was extracted with 3×30 mL of dichloromethane and the organic layers combined. The resulting mixture was washed with 1×50 mL of sodium chloride. The mixture was dried over anhydrous sodium sulfate and concentrated under vacuum. This resulted in 1.9 g (52%) of (3R,5S,6S,8R,9S,10R,13S,14S,17S)-17-(2-bromoacetyl)-3-hydroxy-10,13-dimethyl-hexadecahydro-1H-cyclopenta[a]phenanthren-6-yl acetate as yellow oil.

¹H-NMR-A24: (CDCl₃, 400 MHz): δ4.675-4.686 (m, 1H), 3.926 (s, 2H), 2.831-2.854 (m, 1H), 2.053 (s, 3H), 0.897 (s, 3H), 0.660 (s, 3H).

### 9. Synthesis of A25

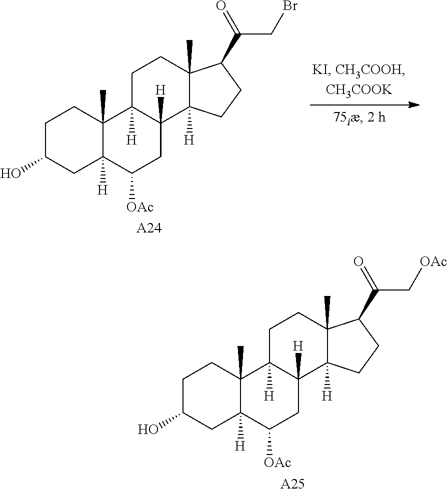

Into a 250-mL round-bottom flask, was placed (3R,5S,6S,8R,9S,10R,13S,14S,17S)-17-(2-bromoacetyl)-3-hydroxy-10,13-dimethyl-hexadecahydro-1H-cyclopenta[a]phenanthren-6-yl acetate (1.9 g, 4.17 mmol, 1.00 equiv), Acetone (76 mL), CH₃COOK (3.19 g, 32.50 mmol, 7.79 equiv), KI (79 mg), acetic acid (3.14 mL). The resulting solution was stirred for 2 h at 75° C. The resulting mixture was concentrated under vacuum. The resulting solution was extracted with 3×20 mL of ethyl acetate and the organic layers combined. The resulting mixture was washed with 1×40 mL of sodium chloride. The mixture was dried over anhydrous sodium sulfate and concentrated under vacuum. This resulted in 1.3 g (72%) of A25 as a white solid.

¹H-NMR-A25: (CDCl₃, 400 MHz): δ4.705 (s, 2H), 4.523-4.565 (m, 1H), 4.117-4.152 (m, 1H), 2.186 (s, 3H), 2.067 (s, 3H), 0.880 (s, 3H), 0.668 (s, 3H).

### 10. Synthesis of 9

Into a 250-mL round-bottom flask purged and maintained with an inert atmosphere of nitrogen, was placed A25(1.3 g, 2.99 mmol, 1.00 equiv), methanol (97.5 mL), 10% K₂CO₃ (32.5 mL). The resulting solution was stirred for 30 min at room temperature. The pH value of the solution was adjusted to 6 with hydrogen chloride (1 mol/L). The resulting mixture was concentrated under vacuum. The resulting solution was extracted with 3×30 mL of ethyl acetate and the organic layers combined. The resulting mixture was washed with 1×40 mL of sodium chloride. The mixture was dried over anhydrous sodium sulfate and concentrated under vacuum. The residue was applied onto a silica gel column with ethyl acetate/petroleum ether (32:68). The crude product (600 mg) was purified by Prep-HPLC with the following conditions (1#waters 2767-5): Column, SunFire Prep C18, 19*150 mm 5 umH PrepC-001(T)18600256819513816414 04; mobile phase, Phase A: water with 0.05% of NH₄HCO₃; Phase B: CH₃CN (20% CH3CN up to 80% in 10 min, up to 100% in 0.1 min, hold 100% in 1.9 min, down to 20% in 0.1 min, hold 20% in 1.9 min); Detector, ELSD. 264.8 mg product was obtained. This resulted in 264.8 mg (22%) of (3R,5S,6S,8R,9S,10R,13S,14S,17S)-3-hydroxy-17-(2-hydroxyacetyl)-10,13-dimethyl-hexadecahydro-1H-cyclopenta[a]phenanthren-6-yl acetate as a white solid.

LC-MS-9: (ES, m/z): 391[M−H]⁻

¹H-NMR-9: (CDCl₃, 400 MHz): δ4.681-4.878 (m, 1H), 4.138 (s, 2H), 4.011-4.024 (m, 1H), 2.029 (s, 3H), 0.928 (s, 3H), 0.665 (s, 3H).

### Synthesis of 4

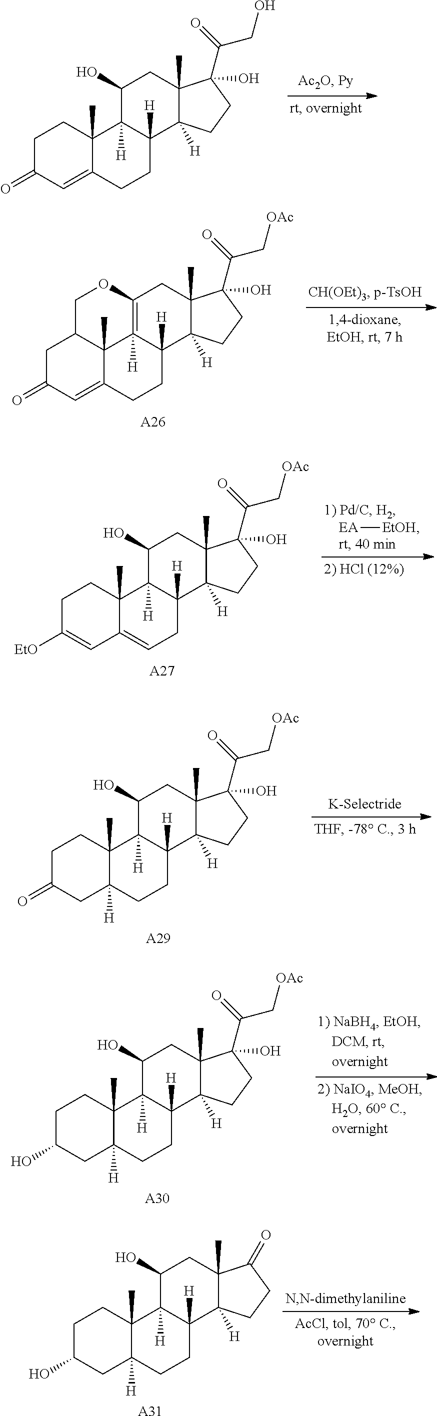

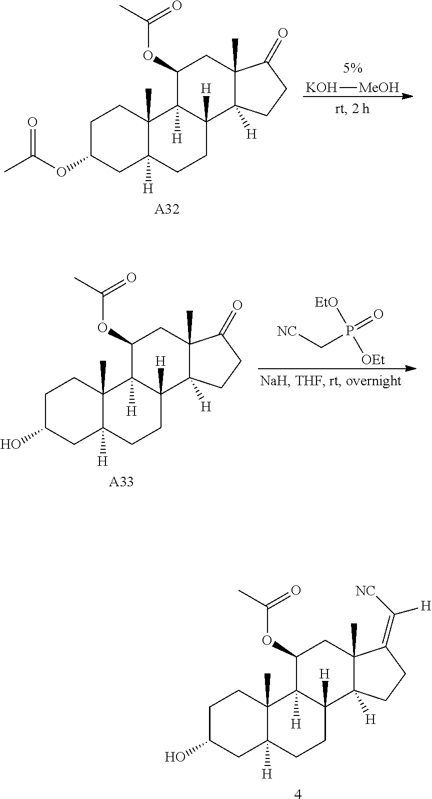

### 1. Synthesis of A26

Into a 500-mL 3-necked round-bottom flask, was placed (8S,9S,10R,11S,13S,14S,17R)-11,17-dihydroxy-17-(2-hydroxyacetyl)-10,13-dimethyl-1,7,8,10,11,12,13,15,16,17-decahydro-2H-cyclopenta[a]phenanthren-3(6H,9H,14H)-one (20 g, 55.18 mmol, 1.00 equiv), Pyridine (200 mL). This was followed by the addition of acetyl acetate (8.453 g, 82.80 mmol, 1.50 equiv) dropwise with stirring at 0° C. The resulting solution was stirred overnight at room temperature. The reaction was then quenched by the addition of 1750 mL of water. The solids were collected by filtration and washed with 500 mL of HCl (1M). The resulting mixture was washed with 3×500 mL of H₂O. The solid was dried by air. This resulted in 20.27 g (91%) of 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl acetate as a white solid.

¹H-NMR-A26: (300 MHz, DMSO): δ 5.564 (s, 1H), 5.417 (s, 1H), 5.054-5.113 (d, J=17.7 Hz, 1H), 4.707-4.765 (d, J=17.4 Hz, 1H), 4.359-4.372 (d, J=3.9 Hz, 1H), 4.263 (s, 1H), 2.169 (s, 3H), 1.364 (s, 3H), 0.760 (s, 3H).

### 2. Synthesis of A27

Into a 250-mL round-bottom flask, was placed 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl acetate (6 g, 14.83 mmol, 1.00 equiv), 1.4-dioxane (55 mL), ethanol (36 mL). This was followed by the addition of CH(OEt)₃ (5.05 g, 2.30 equiv) at 10° C. To this was added 4-methylbenzene-1-sulfonic acid (145 mg, 0.84 mmol, 0.06 equiv). The resulting solution was stirred for 7 h at room temperature. The reaction was then quenched by the addition of 50 mL of potassium carbonate (10%, aq.). The resulting solution was extracted with 3×200 mL of dichloromethane and the organic layers combined and dried over anhydrous sodium sulfate and concentrated under vacuum. The residue was applied onto a silica gel column with ethyl acetate/petroleum ether (½). This resulted in 3 g (47%) of 2-((8S,9S,10R,11S,13S,14S,17R)-3-ethoxy-11,17-dihydroxy-10,13-dimethyl-2,7,8,9,10,11,12,13,14,15,16,17-dodecahydro-1H-cyclopenta[a]phenanthren-17-yl)-2-oxoethyl acetate as a yellow solid.

LC-MS-A27: 433[M+H]⁺

¹H-NMR-A27: (300 MHz, CDCl₃): δ 5.692 (s, 1H), 5.042 (d, J=17.1 Hz, 1H), 4.853 (d, J=17.4 Hz, 1H), 4.488 (d, J=2.7 Hz, 1H), 3.766-3.696 (m, 2H), 2.187 (s, 3H), 1.418 (s, 3H), 1.276-1.229 (t, 3H), 0.978 (s, 3H).

### 3. Synthesis of A29

Into a 250-mL round-bottom flask, was placed 2-((8S,9S,10R,11S,13S,14S,17R)-3-ethoxy-11,17-dihydroxy-10,13-dimethyl-2,7,8,9,10,11,12,13,14,15,16,17-dodecahydro-1H-cyclopenta[a]phenanthren-17-yl)-2-oxoethyl acetate (2.0 g, 4.62 mmol, 1.00 equiv), ethyl acetate (40 mL), ethanol (40 mL), 10% Pd/C (200 mg). To the above hydrogen was introduced in. The resulting solution was stirred for 40 min at room temperature. The solids were filtered out. The solution was then quenched by the addition of 100 mL of HCl (12%, aq.). The resulting solution was extracted with 3×200 mL of ethyl acetate and the organic layers combined. The resulting mixture was washed with 1×200 mL of sodium bicarbonate (10%, aq.). The resulting mixture was washed with 1×200 mL of sodium chloride. The mixture was dried over anhydrous sodium sulfate and concentrated under vacuum. The residue was applied onto a silica gel column with ethyl acetate/petroleum ether (½). This resulted in 700 mg (37%) of 2-((5S,8S,9S,10S,11S,13S,14S,17R)-11,17-dihydroxy-10,13-dimethyl-3-oxo-hexadecahydro-1H-cyclopenta[a]phenanthren-17-yl)-2-oxoethyl acetate as a white solid.

¹H-NMR-A29: (300 MHz, CDCl3): δ 5.029-5.088 (d, J=17.7 Hz, 1H), 4.810-4.868 (d, J=17.4 Hz, 1H), 4.60 (s, 1H), 2.182 (s, 3H), 1.268 (s, 3H), 0.94 (s, 3H)

### 4. Synthesis of A30

Into a 50-mL 3-necked round-bottom flask purged and maintained with an inert atmosphere of nitrogen, was placed 2-((5S,8S,9S,10S,11S,13S,14S,17R)-11,17-dihydroxy-10,13-dimethyl-3-oxo-hexadecahydro-1H-cyclopenta[a]phenanthren-17-yl)-2-oxoethyl acetate (3.0 g, 7.38 mmol, 1.00 equiv), tetrahydrofuran (30 mL). This was followed by the addition of K-Selectride (10.95 mL, 1.50 equiv) dropwise with stirring at −78° C. The resulting solution was stirred for 3 h at −78° C. The reaction was then quenched by the addition of 2 mL of H₂O₂. The resulting solution was extracted with 3×200 mL of ethyl acetate and the organic layers combined. The resulting mixture was washed with 1×200 mL of sodium chloride. The mixture was dried over anhydrous sodium sulfate and concentrated under vacuum. This resulted in 3.8 g (crude) of 2-oxo-2-((3R,5S,8S,9S,10S,11S,13S,14S,17R)-3,11,17-trihydroxy-10,13-dimethyl-hexadecahydro-1H-cyclopenta[a]phenanthren-17-yl)ethyl acetate as a colorless oil.

### 5. Synthesis of A31

Into a 500-mL round-bottom flask, was placed 2-oxo-2-((3R,5S,8S,9S,10S,11S,13S,14S,17R)-3,11,17-trihydroxy-10,13-dimethyl-hexadecahydro-1H-cyclopenta[a]phenanthren-17-yl)ethyl acetate (3.8 g, 9.30 mmol, 1.00 equiv), dichloromethane (20 mL), ethanol (20 mL). This was followed by the addition of NaBH₄ (707.8 g, 18.71 mol, 2.00 equiv) at 10° C. The resulting solution was stirred overnight at 25° C. To this was added 40 mL of acetone (50% in water), the resulting solution was stirred 1 h at 25° C. This was followed by the addition of methanol (150 mL), H₂O (150 mL), NaIO₄ (7.97 g, 4.00 equiv). The resulting solution was allowed to react, with stirring, for an additional overnight at 60° C. The resulting solution was extracted with 3×200 mL of dichloromethane and the organic layers combined. The resulting mixture was washed with 1×200 mL of sodium chloride. The mixture was dried over anhydrous sodium sulfate and concentrated under vacuum. The residue was applied onto a silica gel column with ethyl acetate/petroleum ether (½). This resulted in 2.0 g (70%) of (3R,5S,8S,9S,10S,11S,13S,14S)-3,11-dihydroxy-10,13-dimethyl-tetradecahydro-2H-cyclopenta[a]phenanthren-17(14H)-one as a white solid.

¹H-NMR-A31: (300 MHz, CDCl₃): δ 4.464 (s, 1H), 4.086 (s, 1H), 1.130 (s, 3H), 1.070 (s, 3H).

### 6. Synthesis of A32

Into a 250-mL round-bottom flask, was placed (3R,5S,8S,9S,10S,11S,13S,14S)-3,11-dihydroxy-10,13-dimethyl-tetradecahydro-2H-cyclopenta[a]phenanthren-17(14H)-one (650 mg, 2.12 mmol, 1.00 equiv), N,N-dimethylaniline (31.2 mL). This was followed by the addition of acetyl chloride (15.6 mL) dropwise with stirring at 0° C. To this was added methylbenzene (46.8 mL). The resulting solution was stirred overnight at 70° C. The reaction was then quenched by the addition of 100 mL of HCl (2M). The resulting solution was extracted with 3×100 mL of ethyl acetate and the organic layers combined. The resulting mixture was washed with 1×100 mL of hydrogen chloride (12%). The resulting mixture was concentrated under vacuum. The crude product (550 mg) was purified by Flash-Prep-HPLC with the following conditions (IntelFlash-1): Column, C18 silica gel, 25 um; mobile phase: water with 0.05% TFA and CH₃CN (70% CH₃CN up to 100% in 20 min, Detector: ELSD. This resulted in 430 mg (52%) of (3R,5S,8S,9S,10S,11S,13S,14S)-10,13-dimethyl-17-oxo-hexadecahydro-1H-cyclopenta[a]phenanthrene-3,11-diyl diacetate as a white solid.

¹H-NMR-A32: (300 MHz, CDCl₃): δ 5.432-5.463 (m, 1H), 4.995 (s, 1H), 2.023 (s, 3H), 2.049 (s, 3H), 0.997 (s, 3H), 0.895 (s, 3H).

### 7. Synthesis of A33

Into a 100-mL round-bottom flask, was placed (3R,5S,8S,9S,10S,11S,13S,14S)-10,13-dimethyl-17-oxo-hexadecahydro-1H-cyclopenta[a]phenanthrene-3,11-diyl diacetate (500 mg, 1.28 mmol, 1.00 equiv), methanol (10 mL). This was followed by the addition of potassium hydroxide (5% in H₂O) dropwise with stirring at 0° C. The resulting solution was stirred for 2 h at room temperature. The reaction was then quenched by the addition of 40 mL of NH₄Cl (10%, aq.). The resulting solution was extracted with 3×150 mL of ethyl acetate and the organic layers combined and dried over anhydrous sodium sulfate and concentrated under vacuum. The residue was applied onto a silica gel column with ethyl acetate/petroleum ether (⅓). This resulted in 370 mg (83%) of (3R,5S,8S,9S,10S,11S,13S,14S)-3-hydroxy-10,13-dimethyl-17-oxo-hexadecahydro-1H-cyclopenta[a]phenanthren-11-yl acetate as a white solid.

¹H-NMR-A33: (300 MHz, CDCl₃): δ 5.456-5.466 (d, J=3 Hz, 1H), 4.049-4.057 (d, J=2.4 Hz, 1H), 2.068 (s, 3H), 0.972 (s, 3H), 0.881 (s, 3H).

### 8. Synthesis of 4

Into a 100-mL round-bottom flask purged and maintained with an inert atmosphere of nitrogen, was placed NaH (60%) (381.6 mg, 15.90 mmol, 9.00 equiv), tetrahydrofuran (40 mL). This was followed by the addition of diethyl (cyanomethyl)phosphonate (1.876 g, 10.59 mmol, 10.00 equiv) dropwise with stirring at 0° C. The resulting solution was stirred 20 min at 0° C. To this was added a solution of (3R,5S,8S,9S,10S,11S,13S,14S)-3-hydroxy-10,13-dimethyl-17-oxo-hexadecahydro-1H-cyclopenta[a]phenanthren-11-yl acetate (370 mg, 1.06 mmol, 1.00 equiv) in tetrahydrofuran (10 mL). The resulting solution was stirred overnight at room temperature. The reaction was then quenched by the addition of 5 mL of NH₄Cl (10%, aq.). The resulting solution was extracted with 4×150 mL of ethyl acetate and the organic layers combined and dried over anhydrous sodium sulfate and concentrated under vacuum. The residue was applied onto a silica gel column with ethyl acetate/petroleum ether (½). The crude product (210 mg) was purified by chiral-HPLC with the following conditions (Prep-chiral-HPLC-004): Column, IC2*25 cm, 5 um Chiral-P(IC)001IC00CJ-LD016; mobile phase, Phase A:Hex-HPLC Phase B:EtOH-HPLC (hold 30% ethanol in 30 min); Detector, UV 220 nm. 50 mg product was obtained. This resulted in 50 mg (13%) of (3R,5S,8S,9S,10S,11S,13S,14S,Z)-17-(cyanomethylene)-3-hydroxy-10,13-dimethyl-hexadecahydro-1H-cyclopenta[a]phenanthren-11-yl acetate as a white solid.

LC-MS-4: (ES, m/z): 394[M+Na]⁺

¹H-NMR-4: (300 MHz, CDCl₃): δ 5.530-5.551 (t, J=3 Hz, 1H), 5.052-5.073 (m, 1H), 4.044-4.052 (d, J=2.4 Hz, 1H), 2.031 (s, 3H), 1.078 (s, 3H), 0.855 (s, 3H).

Synthesis of 1

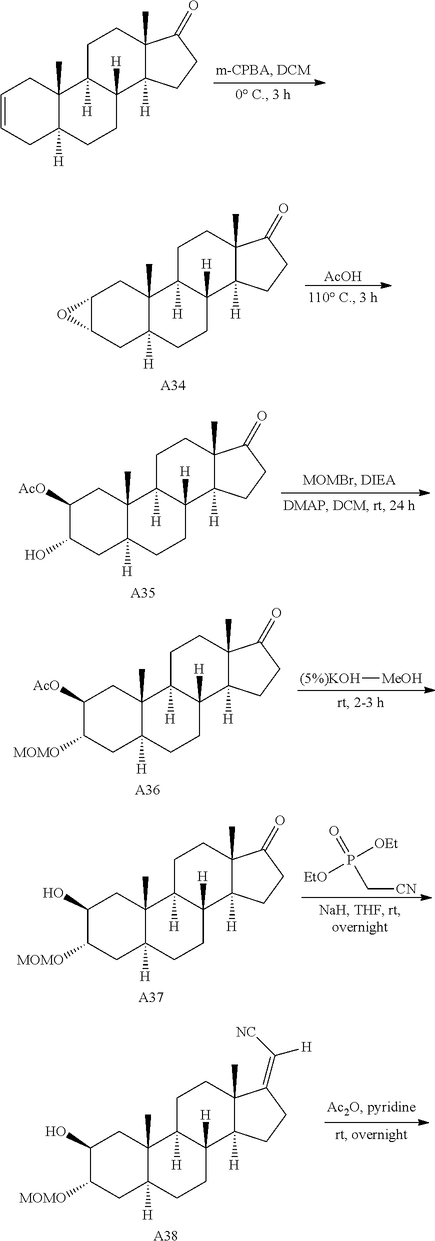

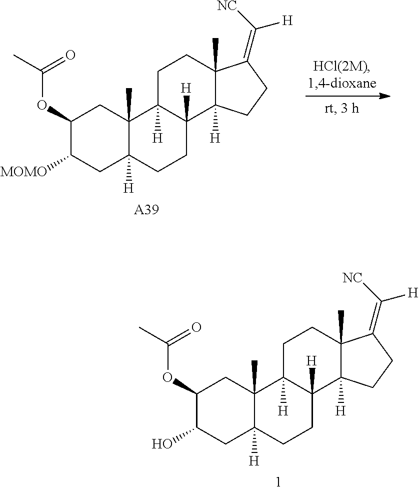

### 1. Synthesis of A34

Into a 3-L 4-necked round-bottom flask, was placed (5S,8R,9S,10S,13S,14S)-10,13-dimethyl-1,5,6,7,8,9,10,11,12,13,15,16-dodecahydro-4H-cyclopenta[a]phenanthren-17(14H)-one (20 g, 73.41 mmol, 1.00 equiv), dichloromethane (1.5 L). This was followed by the addition of m-CPBA (19.8 g, 114.74 mmol, 1.56 equiv) in several batches. The resulting solution was stirred for 3 h at 0° C. in a water/ice bath. The resulting mixture was washed with 2×800 mL of potassium carbonate. The resulting mixture was washed with 300 mL of brine. The mixture was dried over anhydrous sodium sulfate. The residue was applied onto a silica gel column with ethyl acetate/petroleum ether (½). This resulted in 18 g (85%) of A34 as a white solid.

### 2. Synthesis of A35

Into a 500-mL round-bottom flask, was placed A34 (20 g, 69.34 mmol, 1.00 equiv), AcOH (200 mL). The resulting solution was stirred for 3 h at 110° C. in an oil bath. The resulting mixture was concentrated under vacuum. The resulting solution was extracted with 3×100 mL of ethyl acetate and the organic layers combined. The resulting mixture was washed with 100 mL of brine. The mixture was dried over anhydrous sodium sulfate. The residue was applied onto a silica gel column with ethyl acetate/petroleum ether (½). This resulted in 12 g (50%) of (2S,3S,5S,8R,9S,10S,13S,14S)-3-hydroxy-10,13-dimethyl-17-oxo-hexadecahydro-1H-cyclopenta[a]phenanthren-2-yl acetate as a white solid.

¹H-NMR-A35: (400 MHz, CDCl₃): δ 4.904 (s, 1H), 3.894-3.889 (d, J=2 Hz, 1H), 2.083 (s, 3H), 0.961 (s, 3H), 0.876 (s, 3H).

### 3. Synthesis of A36

Into a 250-mL round-bottom flask, was placed (2S,3S,5S,8R,9S,10S,13S,14S)-3-hydroxy-10,13-dimethyl-17-oxo-hexadecahydro-1H-cyclopenta[a]phenanthren-2-yl acetate (8 g, 22.96 mmol, 1.00 equiv), DIEA (15.6 mL, 4.00 equiv), 4-dimethylaminopyridine (595 mg, 4.87 mmol, 0.20 equiv), dichloromethane (100 mL). This was followed by the addition of MOMBr (4.8 mL, 2.50 equiv) dropwise with stirring at 0° C. The resulting solution was stirred overnight at room temperature. The resulting mixture was washed with 2×200 mL of H₂O. The resulting solution was extracted with 3×100 mL of dichloromethane and the organic layers combined. The resulting mixture was washed with 100 mL of brine. The mixture was dried over anhydrous sodium sulfate. The residue was applied onto a silica gel column with ethyl acetate/petroleum ether (½). This resulted in 7 g (78%) of (2S,3S,5S,8R,9S,10S,13S,14S)-3-(methoxymethoxy)-10,13-dimethyl-17-oxo-hexadecahydro-1H-cyclopenta[a]phenanthren-2-yl acetate as yellow oil.

¹H-NMR-A36: (300 MHz, CDCl₃): δ 4.972 (s, 1H), 4.706-4.646 (m, 2H), 3.699-3.692 (d, J=2.1 Hz, 1H), 3.378 (s, 3H), 2.098 (s, 3H), 0.980 (s, 3H), 0.853 (s, 3H).

### 4. Synthesis of A37

Into a 250-mL round-bottom flask, was placed (2S,3S,5S,8R,9S,10S,13S,14S)-3-(methoxymethoxy)-10,13-dimethyl-17-oxo-hexadecahydro-1H-cyclopenta[a]phenanthren-2-yl acetate (7.0 g, 17.83 mmol, 1.00 equiv), methanol (35 mL). This was followed by the addition of a solution of potassium hydroxide (3.5 g, 6.24 mmol, 0.35 equiv) in methanol (35 mL) in several batches at 0° C. The resulting solution was stirred for 1 overnight at 25° C. The reaction was then quenched by the addition of 100 mL of saturated NH₄Cl (aq) (100 mL). The resulting mixture was concentrated under vacuum. The resulting solution was extracted with 3×100 mL of ethyl acetate and the organic layers combined and dried over anhydrous sodium sulfate. The residue was applied onto a silica gel column with ethyl acetate/petroleum ether (30:100). This resulted in 4.3 g (69%) of (2S,3S,5S,8R,9S,10S,13S,14S)-2-hydroxy-3-(methoxymethoxy)-10,13-dimethyl-tetradecahydro-2H-cyclopenta[a]phenanthren-17(14H)-one as a white solid.

¹H-NMR-A37: (300 MHz, CDCl₃): δ 4.707-4.647 (m, 2H), 4.003-3.994 (d, J=2.7 Hz, 1H), 3.691-3.682 (d, J=2.7 Hz, 1H), 3.395-3.377 (d, J=5.4 Hz, 3H), 1.027 (s, 3H), 0.868 (s, 3H).

### 5. Synthesis of A38

Into a 250-mL 3-necked round-bottom flask purged and maintained with an inert atmosphere of nitrogen, was placed sodium hydride (1.4 g, 58.33 mmol, 4.90 equiv), tetrahydrofuran (150 mL). This was followed by the addition of diethyl (cyanomethyl)phosphonate (6.7 g, 37.82 mmol, 5.00 equiv) dropwise with stirring at 0° C. To this was added a solution of (2S,3S,5S,8R,9S,10S,13S,14S)-2-hydroxy-3-(methoxymethoxy)-10,13-dimethyl-tetradecahydro-2H-cyclopenta[a]phenanthren-17(14H)-one (2 g, 5.71 mmol, 1.00 equiv). The resulting solution was stirred overnight at room temperature. The resulting mixture was concentrated under vacuum. The resulting solution was extracted with 3×100 mL of ethyl acetate and the organic layers combined. The resulting mixture was washed with ×100 mL of brine. The mixture was dried over anhydrous sodium sulfate. The residue was applied onto a silica gel column with ethyl acetate/petroleum ether (½). This resulted in 1.8 g (84%) of (Z)-2-((2S,3S,5S,8R,9S,10S,13S,14S)-2-hydroxy-3-(methoxymethoxy)-10,13-dimethyl-tetradecahydro-2H-cyclopenta[a]phenanthren-17(14H)-ylidene)acetonitrile as a white solid.

¹H-NMR-A38: (300 MHz, CDCl₃): δ 5.087 (s, 1H), 4.699-4.641 (m, 2H), 4.000-3.991 (d, J=2.7 Hz, 1H), 3.687-3.678 (d, J=2.7 Hz, 1H), 3.371 (s, 3H), 1.013-0.988 (d, J=7.5 Hz, 3H), 0.968-0.946 (d, J=6.6 Hz, 3H).

### 6. Synthesis of A39

Into a 50-mL round-bottom flask, was placed (Z)-2-((2S,3S,5S,8R,9S,10S,13S,14S)-2-hydroxy-3-(methoxymethoxy)-10,13-dimethyl-tetradecahydro-2H-cyclopenta[a]phenanthren-17(14H)-ylidene)acetonitrile (400 mg, 1.07 mmol, 1.00 equiv), pyridine (25 mL), acetic anhydride (220 mg, 2.00 equiv). The resulting solution was stirred overnight at room temperature. The resulting mixture was concentrated under vacuum. The resulting solution was extracted with 3×100 mL of ethyl acetate and the organic layers combined. The resulting mixture was washed with 100 mL of brine. The mixture was dried over anhydrous sodium sulfate. The residue was applied onto a silica gel column with dichloromethane/methanol (20/1). This resulted in 350 mg (79%) of (2S,3S,5S,8R,9S,10S,13S,14S,Z)-17-(cyanomethylene)-3-(methoxymethoxy)-10,13-dimethyl-hexadecahydro-1H-cyclopenta[a]phenanthren-2-yl acetate as a white solid.

¹H-NMR-A39: (300 MHz, CDCl₃): δ 4.987-4.970 (t, J=2.4 Hz, 2H), 4.699-4.641 (m, 2H), 3.695-3.687 (d, J=2.4 Hz, 2H), 3.372 (s, 3H), 2.034 (s, 3H), 0.925 (s, 3H), 0.823 (s, 3H).

### 7. Synthesis of 1

Into a 50-mL round-bottom flask, was placed (2S,3S,5S,8R,9S,10S,13S,14S,Z)-17-(cyanomethylene)-3-(methoxymethoxy)-10,13-dimethyl-hexadecahydro-1H-cyclopenta[a]phenanthren-2-yl acetate (350 mg, 0.84 mmol, 1.00 equiv), hydrogen chloride (30 mL), 1,4-dioxane (4 mL). The resulting solution was stirred for 3 h at room temperature. The resulting solution was diluted with 50 mL of H₂O. The resulting solution was extracted with 3×50 mL of dichloromethane and the organic layers combined. The resulting mixture was washed with 2×50 mL of sodium bicarbonate. The resulting mixture was washed with 1×100 mL of brine. The mixture was dried over anhydrous sodium sulfate. The residue was applied onto a silica gel column with ethyl acetate/petroleum ether (½). This resulted in 174.4 mg (55%) of (2S,3S,5S,8R,9S,10S,13S,14S,Z)-17-(cyanomethylene)-3-hydroxy-10,13-dimethyl-hexadecahydro-1H-cyclopenta[a]phenanthren-2-yl acetate as a white solid.

LC-MS-1: (ES, m/z): 743[2M+1]⁺

¹H-NMR-1: (300 MHz, CDCl₃): δ 5.096-5.082 (t, J=1.8 Hz, 1H), 4.893-4.885 (d, J=2.4 Hz, 1H), 3.871-3.863 (d, J=2.4 Hz, 1H), 2.050 (s, 3H), 0.940-0.933 (d, J=2.1 Hz, 6H).

### Synthesis of 3

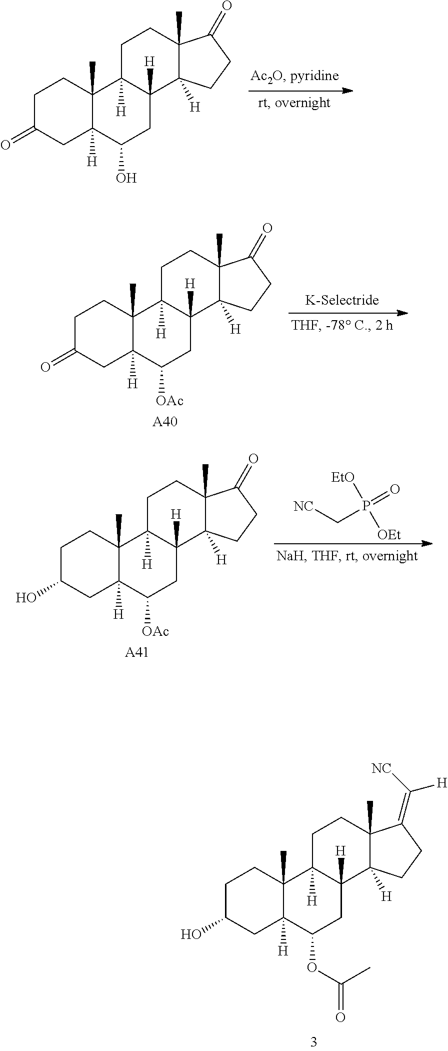

### 1. Synthesis of A40

Into a 250-mL 3-necked round-bottom flask, was placed (5S,6S,8R,9S,10R,13S,14S)-6-hydroxy-10,13-dimethyl-dodecahydro-2H-cyclopenta[a]phenanthrene-3,17(4H,14H)-dione (8.3 g, 27.26 mmol, 1.00 equiv), pyridine (100 mL), acetic anhydride (6.5 g, 63.67 mmol, 2.34 equiv). The resulting solution was stirred for 24 h at 25° C. The resulting solution was diluted with 300 mL of DCM. The resulting mixture was washed with 1×300 mL of hydrogen chloride (1M). The resulting mixture was washed with 1×300 mL of brine. The mixture was dried over anhydrous sodium sulfate. The residue was applied onto a silica gel column with ethyl acetate/petroleum ether (1:1). This resulted in 7.33 g (78%) of (5S,6S,8R,9S,10R,13S,14S)-10,13-dimethyl-3,17-dioxo-hexadecahydro-1H-cyclopenta[a]phenanthren-6-yl acetate as a yellow solid.

¹H-NMR-A40: (300 MHz, CDCl3): δ 4.800-4.741 (m, 1H), 2.046 (s, 3H), 1.113 (s, 3H) 0.894 (s, 1H).

### 2. Synthesis of A41

Into a 250-mL 3-necked round-bottom flask purged and maintained with an inert atmosphere of nitrogen, was placed (5S,6S,8R,9S,10R,13S,14S)-10,13-dimethyl-3,17-dioxo-hexadecahydro-1H-cyclopenta[a]phenanthren-6-yl acetate (7.3 g, 21.07 mmol, 1.00 equiv), tetrahydrofuran (150 mL). This was followed by the addition of a solution of K-Selectrade (1M, 23.3 mL) in tetrahydrofuran dropwise with stirring at −78° C. The resulting solution was stirred for 2 h at −78° C. The reaction was then quenched by the addition of 20 mL of H₂O₂. The resulting mixture was concentrated under vacuum. The resulting solution was diluted with 200 mL of brine. The resulting solution was extracted with 3×250 mL of dichloromethane and the organic layers combined and dried over anhydrous sodium sulfate and concentrated under vacuum. The residue was applied onto a silica gel column with ethyl acetate/petroleum ether (1:1). This resulted in 4.89 g (67%) of (3R,5S,6S,8R,9S,10R,13S,14S)-3-hydroxy-10,13-dimethyl-17-oxo-hexadecahydro-1H-cyclopenta[a]phenanthren-6-yl acetate as a white solid.

LC-MS-A41: (ES, m/z): 697[2M+H]⁺

¹H-NMR-A41: (400 MHz, CDCl₃): δ 4.701-4.689 (d, J=4.8 Hz, 1H), 4.127-4.115 (t, J=2.4 Hz, 1H), 0.885-0.872 (d, J=5.2 Hz, 6H).

### 3. Synthesis of 3

Into a 25-mL round-bottom flask, was placed sodium hydride (151 mg, 3.77 mmol, 5.26 equiv), tetrahydrofuran (10 mL). This was followed by the addition of diethyl (cyanomethyl)phosphonate (738 mg, 4.17 mmol, 5.80 equiv) dropwise with stirring at 0° C. To this was added a solution of (3R,5S,6S,8R,9S,10R,13S,14S)-3-hydroxy-10,13-dimethyl-17-oxo-hexadecahydro-1H-cyclopenta[a]phenanthren-6-yl acetate (220 mg, 0.72 mmol, 1.00 equiv) in tetrahydrofuran (1 mL) dropwise with stirring. The resulting solution was stirred for overnight at 25° C. The reaction was then quenched by the addition of 5 mL of water. The resulting solution was diluted with 50 mL of brine. The resulting solution was extracted with 4×50 mL of ethyl acetate and the organic layers combined and dried over anhydrous sodium sulfate and concentrated under vacuum. The residue was applied onto a silica gel column with ethyl acetate/petroleum ether (1:1). The crude product (195 mg) was purified by Chiral-Prep-HPLC with the following conditions (2#-Gilson Gx 281(HPLC-09)): Column, Phenomenex Lux 5 u Cellulose-4, 2.12*25.5 um; mobile phase, Hex and IPA (hold 30.0% IPA in 8 min); Detector, UV 220 nm. 46 mg product was obtained. This resulted in 46 mg (17%) of (3R,5S,6S,8R,9S,10R,13S,14S,Z)-17-(cyanomethylene)-3-hydroxy-10,13-dimethyl-hexadecahydro-1H-cyclopenta[a]phenanthren-6-yl acetate as a light yellow solid.

LC-MS-3: (ES, m/z): 394[M+Na]⁺, 743[2M+1]⁺

¹H-NMR-3: (300 MHz, CDCl₃): δ 5.107 (s, 1H), 4.708-4.619 (m, 1H), 4.112 (s, 1H), 2.644-2.567 (m, 2H), 2.445-2.330 (m, 1H).

### Synthesis of 5

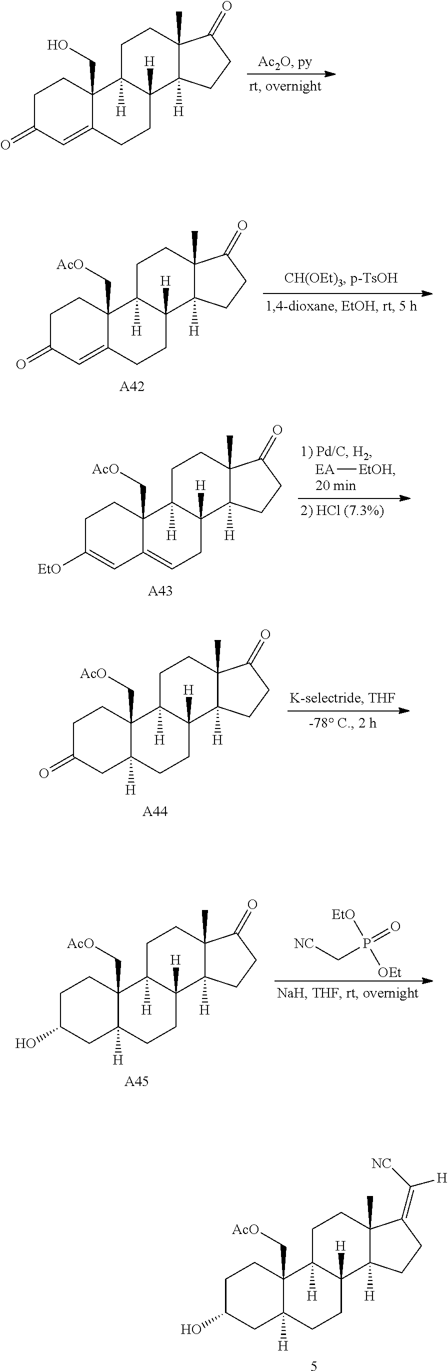

### 1. Synthesis of A42

Into a 1000-mL round-bottom flask, was placed a solution of (8R,9S,10S,13S,14S)-10-(hydroxymethyl)-13-methyl-1,7,8,9,10,11,12,13,15,16-decahydro-2H-cyclopenta[a]phenanthrene-3,17(6H,14H)-dione (42 g, 138.89 mmol, 1.00 equiv) in pyridine (300 mL). This was followed by the addition of acetic anhydride (38.37 g, 376.18 mmol, 2.00 equiv) dropwise with stirring at 0° C. The resulting solution was stirred for 1 overnight at 25° C. The resulting mixture was concentrated under vacuum. The reaction was then quenched by the addition of 500 mL of water. The resulting solution was extracted with 3×250 mL of ethyl acetate and the organic layers combined. The resulting mixture was washed with 2×200 mL of hydrogen chloride (1M). The resulting mixture was washed with 1×200 mL of salt water. The mixture was dried over anhydrous sodium sulfate and concentrated under vacuum. This resulted in 46 g (96%) of ((8R,9S,10S,13S,14S)-13-methyl-3,17-dioxo-2,3,6,7,8,9,10,11,12,13,14,15,16,17-tetradecahydro-1H-cyclopenta[a]phenanthren-10-yl)methylacetate as yellow oil.

LC-MS-A42: (ES, m/z): 345[M+H]⁺

H-NMR-A42: ¹H NMR (300 MHz, CDCl₃, ppm): δ 5.936 (s, 1H), 4.665-4.706 (m, 1H), 4.161-4.199 (d, J=11.4 Hz, 1H), 2.006 (s, 3H), 0.922 (s, 3H).

### 2. Synthesis of A43

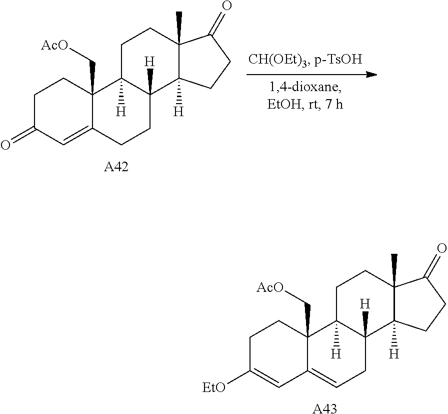

Into a 1000-mL round-bottom flask, was placed ((8R,9S,10S,13S,14S)-13-methyl-3,17-dioxo-2,3,6,7,8,9,10,11,12,13,14,15,16,17-tetradecahydro-1H-cyclopenta[a]phenanthren-10-yl)methyl acetate (33 g, 95.81 mmol, 1.00 equiv), dioxane (330 mL), ethanol (30 mL), p-TsOH (300 g, 0.02 equiv), CH(OEt)3 (33 g, 2.20 equiv). The resulting solution was stirred for 2 h at room temperature. The reaction was then quenched by the addition of 300 mL of potassium carbonate. The resulting solution was extracted with 2×500 mL of ethyl acetate and the organic layers combined and dried over anhydrous sodium sulfate and concentrated under vacuum. The residue was applied onto a silica gel column with ethyl acetate/petroleum ether (⅙). This resulted in 22 g (62%) of ((8R,9S,10S,13S,14S)-3-ethoxy-13-methyl-17-oxo-2,7,8,9,10,11,12,13,14,15,16,17-dodecahydro-1H-cyclopenta[a]phenanthren-10-yl)methyl acetate as a white solid.

H-NMR-A43: ¹H NMR (300 MHz, CDCl₃, ppm): δ 5.866 (s, 1H), 4.599-4.637 (d, J=11.4 Hz, 1H), 4.091-4.128 (d, J=11.1 Hz, 1H), 3.620-3.690 (m, 2H), 2.068 (s, 3H), 0.852 (s, 3H).

### 3. Synthesis of A44

Into a 500-mL round-bottom flask, was placed ((8R,9S,10S,13S,14S)-3-ethoxy-13-methyl-17-oxo-2,7,8,9,10,11,12,13,14,15,16,17-dodecahydro-1H-cyclopenta[a]phenanthren-10-yl)methyl acetate (9 g, 24.16 mmol, 1.00 equiv), ethyl acetate (135 mL), ethanol (63 mL), 10% Palladium carbon (900 mg). To the above hydrogen was introduced in. The resulting solution was stirred for 20 min at room temperature. The solids were filtered out. The pH value of the solution was adjusted to 1 with hydrogen chloride (2 mol/L) (500 mL). The resulting solution was extracted with 4×500 mL of ethyl acetate and the organic layers combined and dried over anhydrous sodium sulfate and concentrated under vacuum. The residue was applied onto a silica gel column with ethyl acetate/petroleum ether (⅕). This resulted in 4.4 g (53%) of ((5S,8R,9S,10R,13S,14S)-13-methyl-3,17-dioxo-hexadecahydro-1H-cyclopenta[a]phenanthren-10-yl)methyl acetate as yellow oil.

H-NMR-A44: ¹H NMR (300 MHz, CDCl₃, ppm): δ 4.390-4.530 (m, 2H), 2.099 (s, 3H), 0.886 (s, 3H)

### 4. Synthesis of A45

Into a 100-mL 3-necked round-bottom flask purged and maintained with an inert atmosphere of nitrogen, was placed ((5S,8R,9S,10R,13S,14S)-13-methyl-3,17-dioxo-hexadecahydro-1H-cyclopenta[a]phenanthren-10-yl)methyl acetate (2 g, 5.77 mmol, 1.00 equiv), tetrahydrofuran (50 g, 693.39 mmol, 120.12 equiv). This was followed by the addition of K-Seletride (6 mL) dropwise with stirring at −78° C. The resulting solution was stirred for 2 h at −78° C. The reaction was then quenched by the addition of 2 mL of H₂O₂. The resulting solution was extracted with 3×150 mL of ethyl acetate and the organic layers combined and dried over anhydrous sodium sulfate and concentrated under vacuum. The residue was applied onto a silica gel column with ethyl acetate/petroleum ether (⅕). This resulted in 780 mg (39%) of ((3R,5S,8R,9S,10R,13S,14S)-3-hydroxy-13-methyl-17-oxo-hexadecahydro-1H-cyclopenta[a]phenanthren-10-yl)methyl acetate as a white solid.

LC-MS-A45: (ES, m/z): 390[M+H+CH₃CN]⁺

H-NMR-A45: ¹H NMR (300 MHz, CDCl₃, ppm): δ 4.206-4.364 (m, 2H), 4.103-4.120 (t, J=2.7 Hz, 1H), 2.073 (s, 3H), 0.858 (s, 3H).

### 5. Synthesis of 5

Into a 100-mL round-bottom flask, was placed sodium hydride (576 mg, 24.00 mmol, 10.72 equiv), tetrahydrofuran (40 mL). This was followed by the addition of diethyl (cyanomethyl)phosphonate (2.8 g, 15.81 mmol, 7.06 equiv) dropwise with stirring at 0° C. To this was added a solution of ((3R,5S,8R,9S,10R,13S,14S)-3-hydroxy-13-methyl-17-oxo-hexadecahydro-1H-cyclopenta[a]phenanthren-10-yl)methyl acetate (780 mg, 2.24 mmol, 1.00 equiv) in tetrahydrofuran dropwise with stirring at 0° C. The resulting solution was stirred overnight at 25° C. The reaction was then quenched by the addition of 50 mL of water. The resulting solution was extracted with 3×50 mL of ethyl acetate and the organic layers combined and dried over anhydrous sodium sulfate and concentrated under vacuum. The residue was applied onto a silica gel column with ethyl acetate/petroleum ether (⅕). The crude product (270 mg) was purified by Chiral-Prep-HPLC with the following conditions (Prep-HPLC-004): Column, Phenomenex Lux 5 u Cellulose-4, 2.12*25.5 um; mobile phase, Hex-HPLC and IPA-HPLC (hold 30% IPA-HPLC in 20 min); Detector, uv 254/220 nm. 67.4 mg product was obtained. This resulted in 67.4 mg (8%) of ((3R,5S,8S,9S,10R,13S,14S,Z)-17-(cyanomethylene)-3-hydroxy-13-methyl-hexadecahydro-1H-cyclopenta[a]phenanthren-10-yl)methyl acetate as white solid.

LC-MS-5: (ES, m/z):354[M−OH]⁺

H-NMR-5: ¹H NMR (300 MHz, CDCl₃, ppm): δ 5.0-5.101 (t, J=2.1 Hz, 1H), 4.189-4.362 (m, 2H), 4.101-4.109 (d, J=2.4 Hz, 1H), 2.073 (s, 3H), 0.945 (s, 3H)

### Synthesis of 11

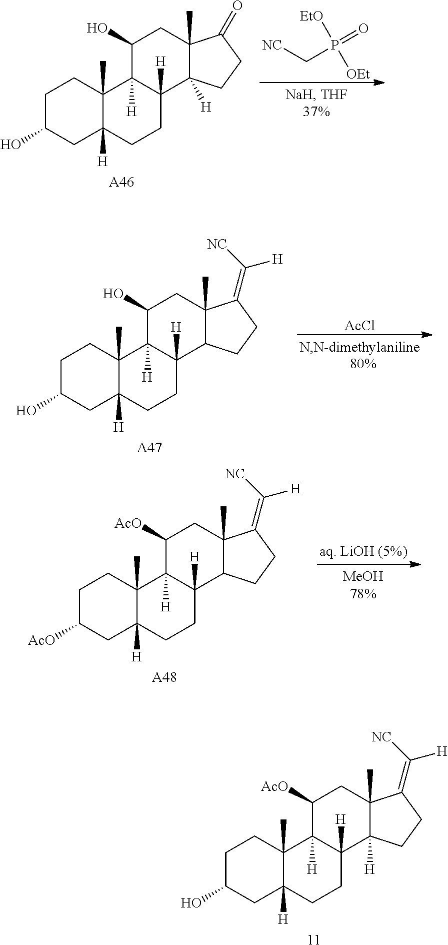

### 1. Preparation of A47

To a solution of NaH (1.3 g, 32.60 mmol) in THF (50 mL) was added diethyl (cyanomethyl)phosphonate (5.77 g, 32.60 mmol) at 0° C. under N₂. The mixture was stirred at 0° C. for 20 minutes. Then the mixture was added dropwise a solution of A46 (1 g, 3.26 mmol) in THF (10 mL). The mixture was stirred at room temperature over night. TLC (petroleumether: ethyl acetate=1:1) showed that the reaction was completely. The reaction was quenched with aqueous NH₄Cl (50 mL). The resulting solution was extracted with 2×100 mL of ethylacetate and the organic layers combined and dried over anhydrous sodium sulfate. The organic phase was concentrated under vacuum to give crude product, which was purified by column chromatography on silica gel (petroleum ether:ethyl acetate=4:1) to give A47 (400 mg, 37%) as white powder.

¹HNMR: (400 MHz, CDCl3) δ 5.07-5.06 (m, 1H), 4.34-4.33 (m, 1H), 3.70-3.63 (m, 1H), 2.71-2.59 (m, 2H), 2.24-2.33 (m, 1H), 1.97-1.90 (m, 3H), 1.78-1.51 (m, 8H), 1.39-1.27 (m, 5H), 1.18-1.16 (m, 6H), 1.13-1.08 (m, 1H).

### 2. Preparation of A48

To a solution of A47 (400 mg, 1.21 mmol) in N,N-dimethylaniline (20 mL) was added AcCl (10 mL). The mixture was stirred at 70° C. over night. TLC (petroleumether:ethyl acetate=3:1) showed that the reaction was completed. The mixture was quenched with H₂O. Then the mixture was extracted with EtOAc (100 mL) and aqueous NaCl (100 mL). The organic phase was dried over Na₂SO₄ and evaporated to give the crude product which was purified by column chromatography on silica gel (petroleum ether:ethyl acetate=20:1) to afford the pure A48 (400 mg, 80%) as a white solid.

¹HNMR: (400 MHz, CDCl3) δ 5.40-5.39 (m, 1H), 5.07-5.06 (m, 1H), 4.75-4.69 (m, 1H), 2.76-2.63 (m, 2H), 2.43-2.36 (m, 1H), 2.04-2.02 (m, 6H), 1.98-1.57 (m, 10H), 1.43-1.16 (m, 7H), 1.07 (s, 3H), 0.98 (s, 3H).

### 3. Preparation of 11

To a solution of A48 (200 mg, 0.48 mmol) in MeOH (5 mL) was added KOH (2 mL, 5% in H₂O). The mixture was stirred at 10° C. for 3 h. TLC (petroleumether:ethyl acetate=3:1) showed that the reaction was completely. The mixture was quenched with aqueous. Then the mixture was extracted with EtOAC (100 mL) and aqueous NaCl (100 mL). The organic phase was dried over Na₂SO₄ and evaporated to give the crude product which was purified by column chromatography on silica gel (petroleum ether: ethylacetate=5:1) to afford 11 (140 mg, 78.5%) as white solid.

¹HNMR: (400 MHz, CDCl3) δ 5.41-5.40 (m, 1H), 5.08-5.07 (m, 1H), 3.68-3.62 (m, 1H), 2.76-2.71 (m, 1H), 2.67-2.60 (m, 1H), 2.44-2.35 (m, 1H), 2.03 (s, 3H), 1.95-1.81 (m, 3H), 1.75-1.52 (m, 8H), 1.40-1.18 (m, 7H), 1.07 (s, 3H), 0.98 (s, 3H).

### Synthesis of 10

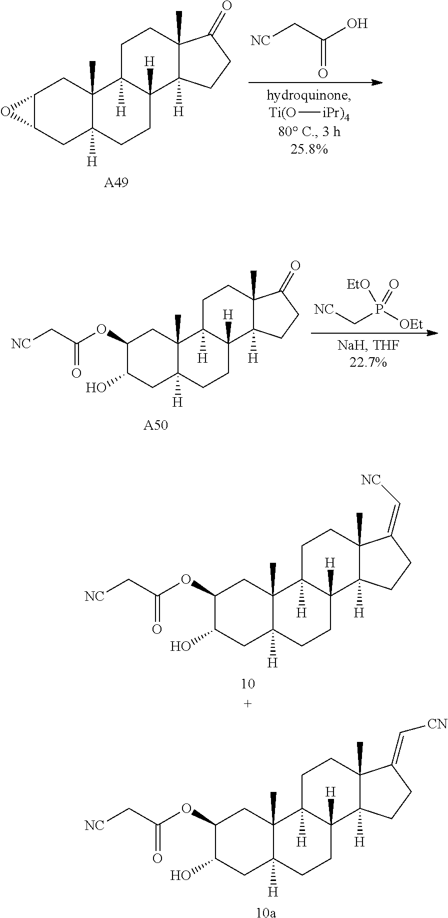

### 1. Preparation of A50

To a mixture of A49 (3 g, 21.69 mmol), tetraisopropoxytitanium (0.15 g, 0.52 mmol) and hydroquinone (0.06 g, 0.52 mmol) in toluene (30 ml) at room temperature under N₂ atmosphere was added 2-cyanoacetic acid (2.65 g, 31.20 mmol) dropwise. The reaction mixture was heated to 80° C. and stirred for 3 hours. The reaction mixture was diluted with EtOAc (30 ml) and aq. NH₄Cl (50 ml), extracted with EtOAc (30 ml). The combined organic layers were washed with H₂O (50 ml) and dried over Na₂SO₄, then concentrated to get the crude product which was purified by column chromatography on silica gel (petroleum ether:ethyl acetate=4:1) to afford the product A50 (1 g, 25.8%).

¹H NMR: (400 MHz, CDCl3) δ 5.05-4.95 (m, 1H), 3.95-3.88 (m, 1H), 3.48 (s, 2H), 2.49-2.41 (m, 1H), 2.11-2.01 (m, 1H), 1.99-1.75 (m, 6H), 1.61-1.16 (m, 10H), 1.09-0.91 (m, 5H), 0.89-0.76 (m, 4H).

### 2. Preparation of 10 and 10a

To a suspension of NaH (0.52 g, 21.69 mmol) in THF (70 ml) at 0° C. under N2 atmosphere was added diethyl (cyanomethyl)phosphonate (4.27 g, 24.10 mmol) dropwise. The reaction mixture was stirred for 20 min and a solution of A50 (900 mg, 2.41 mmol) in THF (10 ml) was added. The reaction mixture was stirred at room temperature over night. The reaction mixture was diluted with EtOAc (30 ml) and aq. NH₄Cl (50 ml), extracted with EtOAc (30 ml). The combined organic layers were washed with H₂O (50 ml) and dried over Na₂SO₄, then concentrated to get the crude product. The crude product was purified by Prep-HPLC and then chiral-HPLC to get the target products of 10 (115 mg, 12.0%) and 10a (102 mg, 10.7%).

### SFC Conditions:

Column: OJ 250 mm*30 mm, 5 um
Mobile phase: A: Supercritical CO₂, B: MeOH (0.1% NH₃.H₂O), A:B=75:25 at 60 ml/min

### Column Temp: 38° C.

### Nozzle Pressure: 100 Bar

### Wavelength: 220 nm

¹H NMR (10): (400 MHz, CDCl3) δ 5.09 (s, 1H), 5.03-4.98 (m, 1H), 3.94-3.89 (m, 1H), 3.46 (s, 2H), 2.66-2.52 (m, 2H), 2.43-2.31 (m, 1H), 1.90-1.16 (m, 19H), 1.08-0.76 (m, 9H).

¹H NMR (10a): (400 MHz, CDCl3) δ 5.00-4.95 (m, 2H), 3.94-3.88 (m, 1H), 3.45 (s, 2H), 2.77-2.66 (m, 1H), 2.62-2.51 (m, 1H), 1.89-1.72 (m, 5H), 1.69-1.05 (m, 17H), 1.04-0.93 (m, 4H), 0.86-0.75 (m, 4H).

### Synthesis of 12

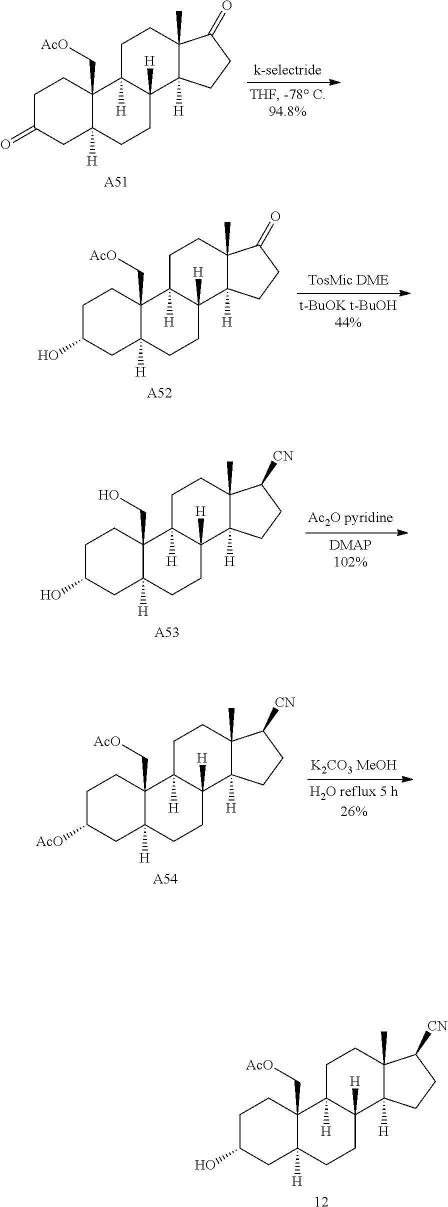

### 1. Preparation of A52

Compound A51 (1.15 g, 3.32 mmol) was dissolved with THF (10 mL). It was evaporated and filled with N₂. Then the mixture was cooled to −78° C. K-selectride (4.98 mL, c=1 mol/L) was added into the solution dropwise. The resulting mixture was stirred at −78° C. for 3 h. The reaction was quenched by the addition of H₂O₂ (5 mL). The resulting mixture was extracted with EtOAc (50 mL*3). The combined organic layer was washed with saturated Na₂SO₃ solution and brine. The combined organic layer was dried over anhydrous Na₂SO₄ and concentrated the solvent to give crude A52 (1.1 g, 94.8%) as a colorless oil.

¹HNMR: (400 MHz, CDCl₃) δ 4.37 (d, J=12.4 Hz, 1H), 4.25 (d, J=12.4 Hz, 1H), 4.15-4.08 (m, 1H), 2.48-2.41 (m, 1H), 2.05 (s, 3H), 1.98-1.88 (m, 2H), 1.86-1.72 (m, 4H), 1.72-1.61 (m, 3H), 1.52-1.46 (m, 2H), 1.38-1.13 (m, 8H), 1.11-1.03 (m, 1H), 0.98-0.88 (m, 1H), 0.85 (s, 3H).

### 2. Preparation of A53

Into a over-dried bottom was added t-BuOH (10 mL) and t-BuOK (3.54 g, 31.6 mmol). It was evaporated and filled with N₂. Compound A52 (1.1 g, 3.16 mmol) in 1,2-dimethoxyethane (5 mL) was added into the suspension. After 30 min, TosMic (1.23 g, 6.32 mmol) in 1,2-dimethoxyethane (5 mL) was added. The mixture became yellow. The resulting mixture was stirred at room temperature (10) for 16 h. TLC (petroleum ether:ethyl acetate=1:1) showed that the reaction was complete. Water was added and the mixture was stirred. Then it was extracted with ethyl acetate (60 mL*3). The combined organic layer was washed with brine. The combined organic layer was dried over anhydrous Na₂SO₄ and concentrated the solvent. The residue was purified by flash chromatography eluting with (petroleum ether:ethyl acetate=4:1) to give A53 (440 mg, 44%) as a pale yellow oil.

¹HNMR: (400 MHz, CDCl₃) δ 4.16-4.11 (m, 1H), 3.93 (d, J=11.6 Hz, 1H), 3.80 (d, J=11.6 Hz, 1H), 2.62-2.26 (m, 0.34H), 2.32-2.26 (m, 0.66H), 2.24-2.10 (m, 1H), 2.05-1.85 (m, 3H), 1.84-1.51 (m, 10H), 1.51-1.31 (m, 3H), 1.26-0.98 (m, 6H), 0.98 (s, 2.1H), 0.86 (s, 1.2H).

### 3. Preparation of A54

To a solution of compound A53 (440 mg, 1.38 mmol) in pyridine (10 mL) was added DMAP (338 mg, 2.76 mmol). Then Ac₂O (566 mg, 5.52 mmol) was added dropwise with stirring. The resulting solution was stirred at room temperature (20° C.) for 16 h. TLC (petroleum ether: ethyl acetate=1:1) showed that the reaction was complete. The mixture was quenched by the addition of water. The mixture was extracted with ethyl acetate (50 mL*3). The combined organic layer was washed with HCl (1 N), saturated NaHCO₃ solution and brine. The combined organic layer was dried over anhydrous Na₂SO₄ and concentrated the solvent to give crude A54 (570 mg, 102%) as a pale yellow oil.

¹HNMR: (400 MHz, CDCl₃) δ 5.08-5.03 (m, 1H), 4.33 (d, J=12.4 Hz, 1H), 4.15 (d, J=12.4 Hz, 1H), 2.28-2.20 (m, 1H), 2.20-2.11 (m, 1H), 2.09 (s, 3H), 2.08-2.02 (m, 4H), 2.01-1.88 (m, 3H), 1.81-1.69 (m, 4H), 1.53-1.48 (m, 1H), 1.46-1.30 (m, 3H), 1.28-1.21 (m, 3H), 1.20-0.96 (m, 5H), 0.93 (s, 3H), 0.92-0.77 (m, 1H).

### 4. Preparation of 12

To a solution of compound A54 (570 mg, 1.42 mmol) in MeOH (15 mL) was added K₂CO₃ (293 mg, 10%) solution. Then the solution was heated until refluxing and maintained the temperature for 5 h. After the mixture was recovered to ambient temperature. The mixture was extracted with EtOAc (50 mL*3). The combined organic layer was dried over anhydrous Na₂SO₄ and concentrated. The residue was purified by flash chromatography eluting with (petroleum ether:ethyl acetate=4:1) to give 12 (135 mg, 26%) as a white solid.

¹H NMR (12) (400 MHz, CDCl₃) δ 4.30 (d, J=12.4 Hz, 1H), 4.23 (d, J=12.4 Hz, 1H), 4.12-4.08 (m, 1H), 2.30-2.22 (m, 1H), 2.18-2.08 (m, 1H), 2.07 (s, 3H), 1.95-1.88 (m, 3H), 1.79-1.49 (m, 4H), 1.66-1.61 (m, 2H), 1.46-0.95 (m, 13H), 0.89 (s, 3H).

### Synthesis of 13

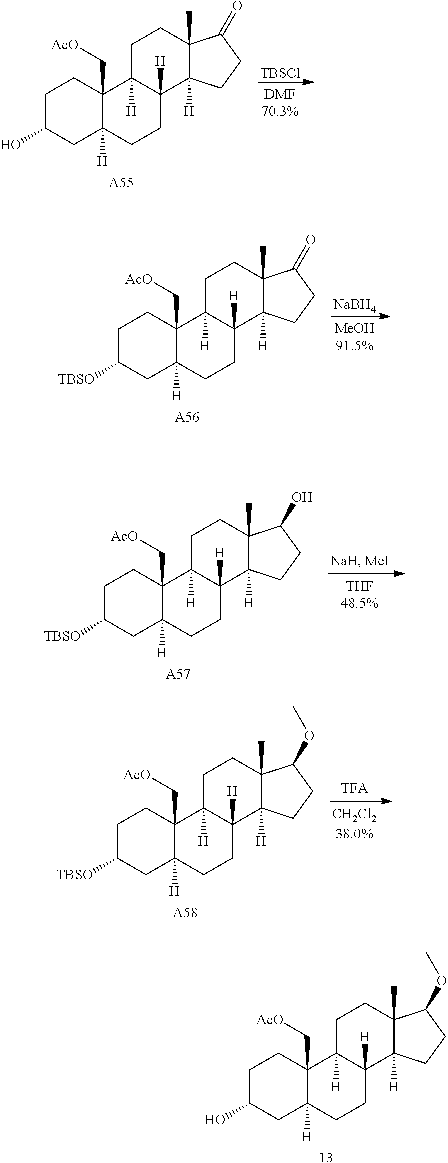

### 1. Preparation of A56

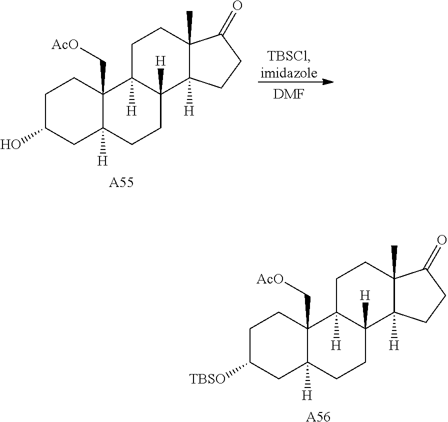

To a solution of A55 (1.5 g, 4.3 mmol) in DMF (10 mL) was added TBSCl (1.3 g, 8.6 mmol) and imidazole (0.44 g, 6.46 mmol). The reaction mixture was stirred at 40° C. for 18 h. TLC indicated the starting material was consumed completely. The mixture was poured into H₂O (50 mL), extracted with EtOAc (20 mL*3). The combined organic layer was dried over anhy. Na₂SO₄, filtered and concentrated. The residue was purified by flash column chromatograph on silica gel (eluent:petroleum ether:ethyl acetate=20:1) to give A56 (1.4 g, 70.5%) as white solid.

¹HNMR: (400 MHz, CDCl3) δ 4.36-4.30 (m, 1H), 4.25-4.18 (m, 1H), 4.05-3.98 (m, 1H), 2.50-2.38 (m, 1H), 2.10-2.05 (m, 4H), 2.00-1.90 (m, 1H), 1.90-1.60 (m, 7H), 1.48-1.16 (m, 9H), 1.16-1.04 (m, 1H), 0.95-0.82 (m, 14H), 0.08-0 (m, 6H).

### 2. Preparation of A57

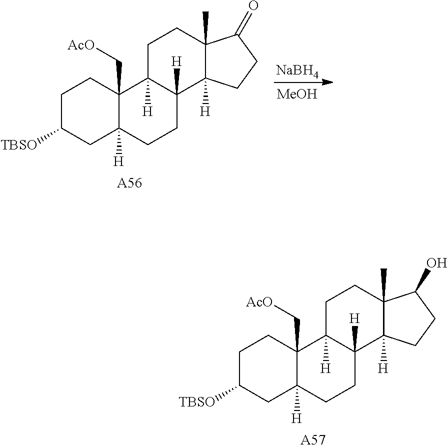

To a solution of A56 (1.2 g, 2.759 mmol) in THF (40 mL) and MeOH (20 mL) under ice bath was added CeCl₃.7H₂O (0.96 g, 3.89 mmol) and NaBH₄ (0.09 g, 2.59 mmol). The reaction mixture was stirred at 0° C. for 30 min. TLC indicated the starting material was consumed completely. The mixture was quenched with H₂O (100 mL), extracted with EtOAc (30 mL*3). The combined organic layer was washed with Sat. NH₄Cl and brine, dried over anhy. Na₂SO₄, filtered and concentrated to give the crude product A57 (1.1 g, 91.5%) as oil, which was used directly in the next step without further purification.

### 3. Preparation of A58

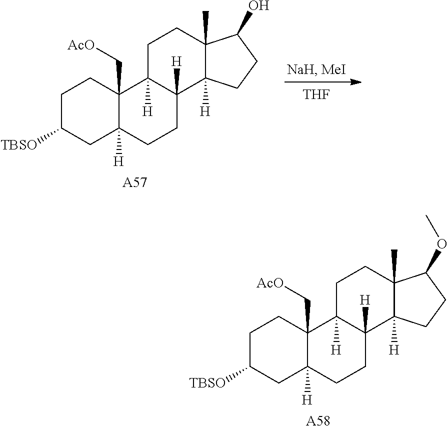

To a suspension of NaH (0.28 g, 11.85 mmol) in THF (3 mL) under ice bath was added a solution of A57 (1.1 g, 2.37 mmol) in THF (7 mL) dropwise. After the mixture stirred for 30 min under ice bath, MeI (3.36 g, 23.7 mmol) was added to it. The reaction mixture was allowed to warm to 20° C. and stirred for 2 h at this temperature. Then it was quenched with Sat. NH₄Cl (aq, 30 mL), extracted with EtOAc (30 mL*2). The combined organic layer was dried and concentrated. The residue was purified by flash column chromatograph on silica gel (eluent:petroleum ether:ethyl acetate=50:1) to give A58 (550 mg, 48.5%) as white solid.

¹HNMR: (400 MHz, CDCl3) δ4.35-4.20 (m, 2H), 4.05-3.95 (m, 1H), 3.34 (s, 3H), 3.24-3.16 (m, 1H), 2.05 (s, 3H), 2.00-1.60 (m, 7H), 1.50-0.95 (m, 15H), 0.90-0.85 (m, 9H), 0.73 (s, 3H), 0.06-0 (m, 6H).

### 4. Preparation of 13

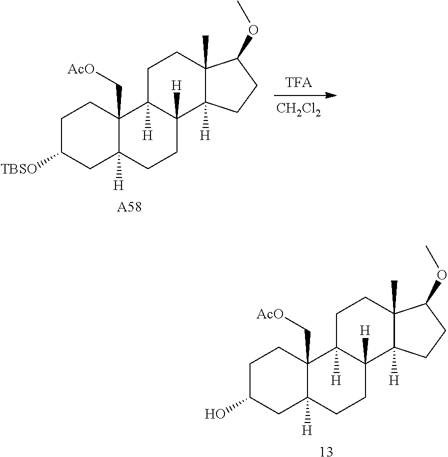

To a solution of A58 (620 mg, 1.29 mmol) in CH₂Cl₂ (20 mL) was added CF₃COOH (3 mL). The reaction mixture was stirred for 15 min at 20° C. Then it was quenched with aq. NaHCO₃ (10%), extracted with EtOAc (20 mL*3). The combined organic layer was dried over anhy. Na₂SO₄, filtered and concentrated. The residue was purified by flash column chromatograph on silica gel (eluent:petroleum ether:ethyl acetate=8:1 to 7:1) to give 13 (178.6 mg, 38.0%) as white powder.

¹HNMR (13): (400 MHz, CDCl3) δ 4.32-4.20 (m, 2H), 4.10-4.06 (m, 1H), 3.34 (s, 3H), 3.21 (t, J=8.4 Hz, 1H), 2.06 (s, 3H), 2.04-1.86 (m, 3H), 1.80-1.05 (m, 17H), 1.00-0.80 (m, 3H), 0.74 (s, 3H).

### Assay Methods

Compounds provided herein can be evaluated using various assays; examples of which are described below.

### Steroid Inhibition of TBPS Binding

TBPS binding assays using rat brain cortical membranes in the presence of 5 μM GABA has been described (Gee et al, J. Pharmacol. Exp. Ther. 1987, 241, 346-353; Hawkinson et al, Mol. Pharmacol. 1994, 46, 977-985).

Briefly, cortices are rapidly removed following decapitation of carbon dioxide-anesthetized Sprague-Dawley rats (200-250 g). The cortices are homogenized in 10 volumes of ice-cold 0.32 M sucrose using a glass/teflon homogenizer and centrifuged at 1500×g for 10 min at 4° C. The resultant supernatants are centrifuged at 10,000×g for 20 min at 4° C. to obtain the P2 pellets. The P2 pellets are resuspended in 200 mM NaCl/50 mM Na—K phosphate pH 7.4 buffer and centrifuged at 10,000×g for 10 min at 4 OC. This ishing procedure is repeated twice and the pellets are resuspended in 10 volumes of buffer. Aliquots (100 μL) of the membrane suspensions are incubated with 2 nM [³⁵S]-TBPS and 5 μL, aliquots of test drug dissolved in dimethyl sulfoxide (DMSO) (final 0.5%) in the presence of 5 μM GABA. The incubation is brought to a final volume of 1.0 mL with buffer. Nonspecific binding is determined in the presence of 2 μM unlabeled TBPS and ranged from 15 to 25%. Following a 90 min incubation at room temp, the assays are terminated by filtration through glass fiber filters (Schleicher and Schuell No. 32) using a cell harvester (Brandel) and rinsed three times with ice-cold buffer. Filter bound radioactivity is measured by liquid scintillation spectrometry. Non-linear curve fitting of the overall data for each drug averaged for each concentration is done using Prism (GraphPad). The data are fit to a partial instead of a full inhibition model if the sum of squares is significantly lower by F-test. Similarly, the data are fit to a two component instead of a one component inhibition model if the sum of squares is significantly lower by F-test. The concentration of test compound producing 50% inhibition (IC₅₀) of specific binding and the maximal extent of inhibition (Imax) are determined for the individual experiments with the same model used for the overall data and then the means+SEM.s of the individual experiments are calculated.

Various compounds are or can be screened to determine their potential as modulators of [³⁵S]-TBPS binding in vitro. These assays are or can be performed in accordance with the above discussed procedures.

### Drug Metabolism and Pharmacokinetics: Half-Life in Human Liver Microsomes (HLM)

Test compounds (1 μM) are incubated with 3.3 mM MgCl₂ and 0.78 mg/mL HLM (HLlOl) in 100 mM potassium phosphate buffer (pH 7.4) at 37° C. on the 96-deep well plate. The reaction mixture is split into two groups, a non-P450 and a P450 group. NADPH is only added to the reaction mixture of the P450 group. An aliquot of samples of P450 group is collected at 0, 10, 30, and 60 min time point, where 0 min time point indicated the time when NADPH was added into the reaction mixture of P450 group. An aliquot of samples of non-P450 group is collected at −10 and 65 min time point. Collected aliquots are extracted with acetonitrile solution containing an internal standard. The precipitated protein is spun down in centrifuge (2000 rpm, 15 min). The compound concentration in supernatant is measured by LC/MS/MS system. The half-life value is obtained by plotting the natural logarithm of the peak area ratio of compounds/internal standard versus time. The slope of the line of best fit through the points yields the rate of metabolism (k). This is converted to a half-life value using the equation: Half-life=In 2/k.

For Table 1, “A” indicates an IC₅₀<10 nM, “B” indicates an IC₅₀ of 10 nM to 50 nM, “C” indicates an IC₅₀>50 nM to 100 nM, “D” indicates an IC₅₀>100 nM to 500 nM, and “E” indicates IC₅₀>500 nM.

For Table 2, “F” indicates Clint <50 and “G” indicates Clint ≧50.

TABLE 1

| | | ³⁵S-TBPS Radioligand |
|---|---|---|
| | | Displacement (Control |
| | Structure | IC₅₀) |
| 1 | 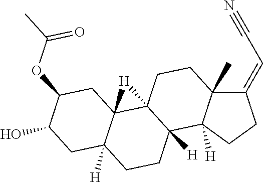 | D |
| 2 | 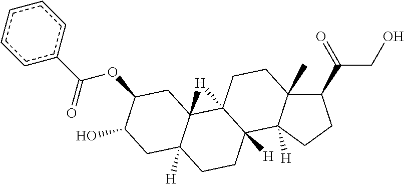 | D |
| 3 | 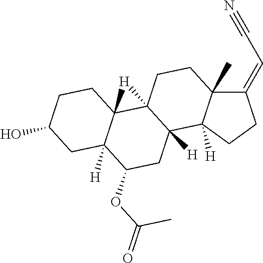 | D |
| 4 | 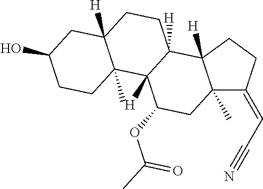 [SMILES: CC(=O)O[C@H]1C[C@]2(C)/C(=C\C#N)CC[C@H]2[C@@H]2CC[C@H]3C[C@H](O)CC[C@]3(C)[C@H]21] | D |
| 5 | 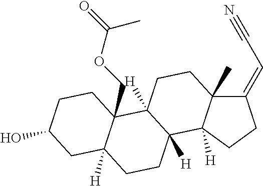 [SMILES: CC(=O)OC[C@]12CC[C@@H](O)C[C@@H]1CC[C@@H]1[C@@H]2CC[C@]2(C)/C(=C\C#N)CC[C@@H]12] | D |
| 6 | 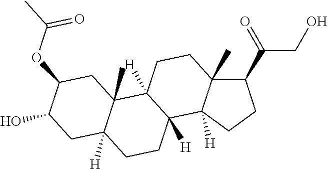 [SMILES: CC(=O)O[C@H]1C[C@@]2(C)[C@@H](CC[C@@H]3[C@@H]2CC[C@]2(C)[C@@H](C(=O)CO)CC[C@@H]32)C[C@@H]1O] | D |
| 7 | 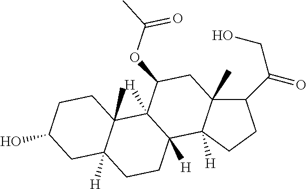 [SMILES: CC(=O)O[C@H]1C[C@]2(C)C(C(=O)CO)CC[C@H]2[C@@H]2CC[C@H]3C[C@H](O)CC[C@]3(C)[C@H]21] | D |
| 8 | 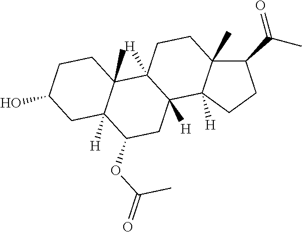 [SMILES: CC(=O)O[C@H]1C[C@@H]2[C@H](CC[C@]3(C)[C@@H](C(C)=O)CC[C@@H]23)[C@@]2(C)CC[C@@H](O)C[C@H]12] | D |
| 9 | 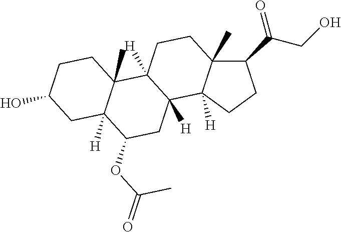 [SMILES: CC(=O)O[C@H]1C[C@@H]2[C@H](CC[C@]3(C)[C@@H](C(=O)CO)CC[C@@H]23)[C@@]2(C)CC[C@@H](O)C[C@H]12] | D |
| 10 | 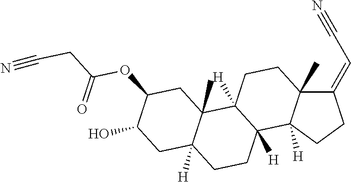 [SMILES: C[C@]12C[C@H](OC(=O)CC#N)[C@@H](O)C[C@@H]1CC[C@@H]1[C@@H]2CC[C@]2(C)/C(=C\C#N)CC[C@@H]12] | |
| 11 | 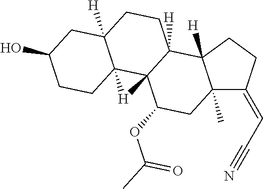 | D |
| 12 | 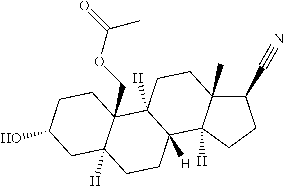 [SMILES: CC(=O)OC[C@]12CC[C@@H](O)C[C@@H]1CC[C@H]1[C@@H]3CC[C@H](C#N)[C@@]3(C)CC[C@@H]12] | |
| 13 | 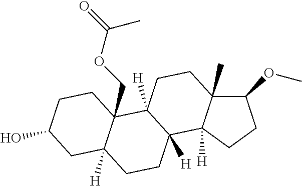 [SMILES: CO[C@H]1CC[C@H]2[C@@H]3CC[C@H]4C[C@H](O)CC[C@]4(COC(C)=O)[C@H]3CC[C@]12C] | D |

TABLE 2

| | Microsome Stability |
|---|---|
| Compound | Human - Clint (Clint) |
| 1 | F |
| 2 | G |
| 3 | F |
| 4 | G |
| 5 | G |
| 6 | G |

1. A compound of Formula (I): 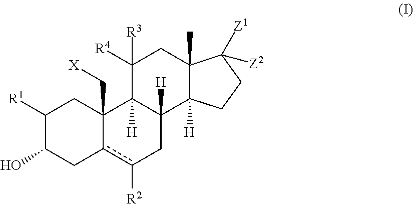
or a pharmaceutically acceptable salt, solvate, stereoisomer, tautomer, and/or isotopic variant thereof;
wherein:
X is hydrogen, halo, —CF₃, —CHF₂, —CH₂F, —NO₂, —CN, —SCN, —OR^{X}, —OC(═O)N(R^{X})₂, —SR^{X}, —SC(═O)N(R^{X})₂, —N(R^{X})₂, —NR^{X}C(═O)R^{X}, —NR^{X}C(═O)OR^{X}, —NR^{X}C(═O)N(R^{X})₂, —NR^{X}SO₂R^{X}, —C(═O)R^{X}, —C(═O)N(R^{X})₂, —S(═O)R^{X}, —S(═O)₂R^{X}, —SO₂N(R^{X})₂, or —OC(═O)R^{E1};
wherein R^{X} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{X} groups are joined to form a substituted or unsubstituted heterocyclic ring;
Z¹ is halo, —CN, —CH₂CN, —CH₂CF₃, —NO₂, —CH₂NO₂, —OR^{Z1b}, —CH₂OR^{Z1b}, —OC(═O)N(R^{Z1b})₂, —SR^{Z1b}, —N(R^{Z1b})₂, —N(OR^{Z1b})(R^{Z1b}), —NR^{Z1b}C(═O)R^{Z1b}, —NR^{Z1b}C(═O)OR^{Z1b}, —NR^{Z1b}C(═O)N(R^{Z1b})₂, —NR^{Z1b}SO₂R^{Z1b}, —C(═O)R^{Z1b}, —CH₂C(═O)R^{Z1b}, —C(═O)OR^{Z1b}, —CH₂C(═O)OR^{Z1b}, —C(═O)N(R^{Z1b})₂, —CH₂C(═O)N(R^{Z1b})₂, —S(═O)R^{Z1b}, —S(═O)₂R^{Z1b}, —SO₂N(R^{Z1b})₂, —P(═O)₂R^{Z1b}, —P(═O)₂OR^{Z1b}, —P(═O)(OR^{Z1b})₂, —P(═O)(R^{Z1b})₂, or —P(═O)(R^{Z1b})(OR^{Z1b}), —C(═O)CH₂OR^{Z1a}, or —C(═O)CH₂N(R^{Z1a})₂; and Z² is hydrogen or —OR^{Z2};
or Z¹ and Z² are joined to form a 3- to 6-membered substituted or unsubstituted heterocyclic ring; an oxo (═O); an oxime ═N(OR^{Z1b}); or an alkenyl group ═CH(Z³), wherein Z³ is —CF₃, —NO₂, —OR^{Z1b}, —C(═O)R^{Z1b}, —C(═O)OR^{Z1b}, or —C(═O)N(R^{Z1b})₂;
or Z¹ and Z² are joined to form an alkenyl group ═CH(CN), wherein CN is in the Z configuration;
wherein each instance of R^{Z1a} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a nitrogen protecting group when attached to a nitrogen atom, —C(═O)R^{Z1b}, —C(═O)OR^{Z1b}, —C(═O)N(R^{Z1b})₂, —C(═O)N(OR^{Z1b})(R^{Z1b}), —S(═O)₂R^{Z1b}, —S(═O)₂OR^{Z1b}, —P(═O)₂R^{Z1b}, —P(═O)₂OR^{Z1b}, —P(═O)(OR^{Z1b})₂, —P(═O)(R^{Z1b})₂, or —P(═O)(R^{Z1b})(OR^{Z1b}), or two R^{Z1a} groups are joined to form a substituted or unsubstituted heterocyclic ring or substituted or unsubstituted heteroaryl ring, and
wherein each instance of R^{Z1b} and R^{Z2} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{Z1b} groups are joined to form a substituted or unsubstituted heterocyclic or substituted or unsubstituted heteroaryl ring, or R^{Z1b} and R^{Z2} are joined to form a substituted or unsubstituted heterocyclic ring;
R¹, R², and R³ are independently selected from the group consisting of hydrogen or —OC(═O)R^{E1};
R⁴ is hydrogen, or R³ and R⁴ are joined to form an oxo (═O) group or an alkenyl group ═C(R^{A3})₂, wherein each instance of R^{A3} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or two R^{a3} groups are joined to form a substituted or unsubstituted carbocyclic ring or substituted or unsubstituted heterocyclic ring; and [symbol]
represents a single or double bond, wherein if [symbol] is a single bond, then the C5 hydrogen is in the alpha or beta configuration;
provided at least one of R¹, R², R³, and X is a group of the formula —OC(═O)R^{E1};
wherein R^{E1} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or —OR^{E2}, and wherein R^{E2} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group.

2. The compound of claim 1, wherein the compound comprises:

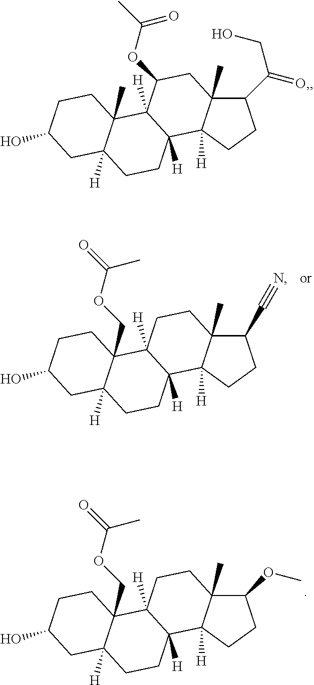 [SMILES: CC(=O)O[C@H]1C[C@]2(C)C(C(=O)CO)CC[C@H]2[C@@H]2CC[C@H]3C[C@H](O)CC[C@]3(C)[C@H]21 ; CC(=O)OC[C@]12CC[C@@H](O)C[C@@H]1CC[C@H]1[C@@H]3CC[C@H](C#N)[C@@]3(C)CC[C@@H]12 ; CO[C@H]1CC[C@H]2[C@@H]3CC[C@H]4C[C@H](O)CC[C@]4(COC(C)=O)[C@H]3CC[C@]12C]

3. The compound of claim 1 of the Formula: 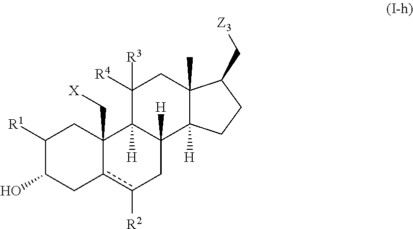
or a pharmaceutically acceptable salt, solvate, stereoisomer, tautomer, and/or isotopic variant thereof.

4. The compound of claim 1 of the Formula: 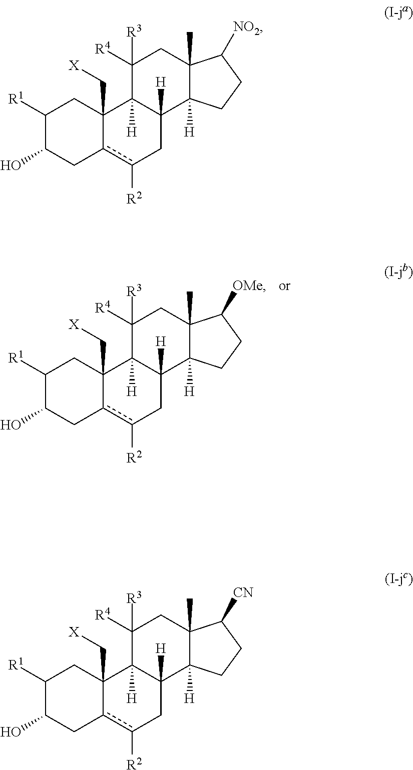
or a pharmaceutically acceptable salt, solvate, stereoisomer, tautomer, and/or isotopic variant thereof.

5. The compound of claim 1 of the Formula: 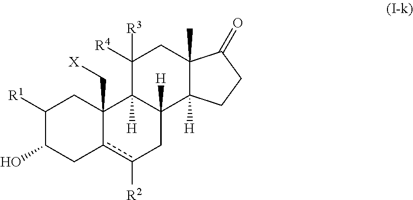
or a pharmaceutically acceptable salt, solvate, stereoisomer, tautomer, and/or isotopic variant thereof.

6. The compound of claim 1 of the Formula: 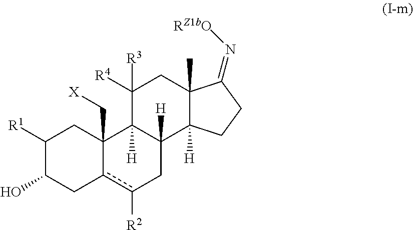
or a pharmaceutically acceptable salt, solvate, stereoisomer, tautomer, and/or isotopic variant thereof.

7. The compound of claim 1 of the Formula: 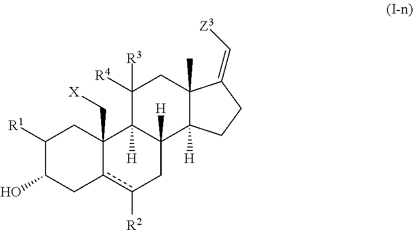
or a pharmaceutically acceptable salt, solvate, stereoisomer, tautomer, and/or isotopic variant thereof.

8. The compound of claim 7, wherein the compound is of the Formula II-n:

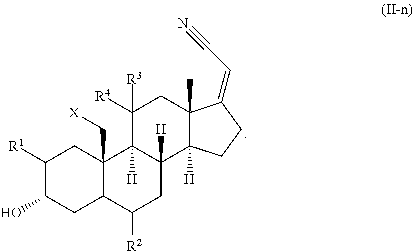

9. The compound of claim 8, wherein the compound comprises:

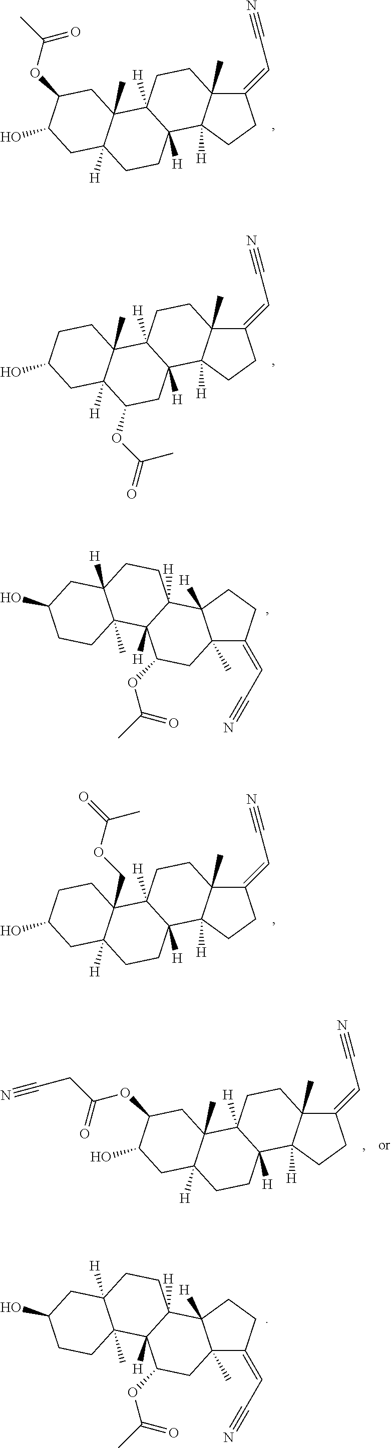

10. The compound of claim 1 of the Formula: 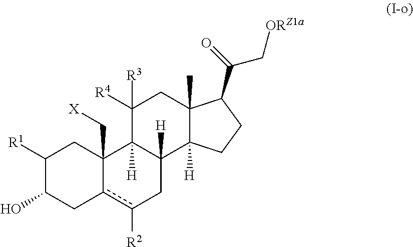
or a pharmaceutically acceptable salt, solvate, stereoisomer, tautomer, and/or isotopic variant thereof.

11. The compound of claim 10, wherein the compound is of the Formula II-o:

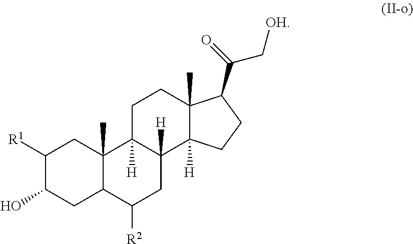

12. The compound of claim 11, wherein the compound comprises:

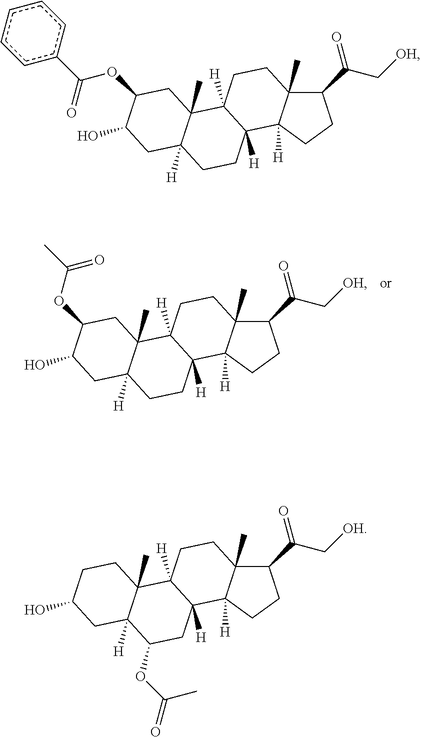

13. The compound of claim 1 of the Formula: 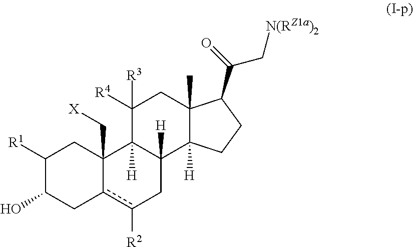
or a pharmaceutically acceptable salt, solvate, stereoisomer, tautomer, and/or isotopic variant thereof.

14. The compound of claim 1, wherein only one of R¹, R², R³, and X is a group of the formula —OC(═O)R^{E1}.

15. The compound of claim 14, wherein R¹ is a group of the formula —OC(═O)R^{E1}.

16. The compound of claim 14, wherein R² is a group of the formula —OC(═O)R^{E1}.

17. The compound of claim 14, wherein R³ is a group of the formula —OC(═O)R^{E1}.

18. The compound of claim 14, wherein X is a group of the formula —OC(═O)R^{E1}.

19. The compound of claim 1, wherein at least two of R¹, R², R³, and X is, independently, a group of the formula —OC(═O)R^{E1}.

20. The compound of claim 1, wherein R^{E1} is —CH₃, —CH₂CN, or phenyl.

21. The compound of claim 20, wherein R^{E1} is —CH₃.

22. The compound of claim 1, wherein the compound is selected from any one of the formulae: 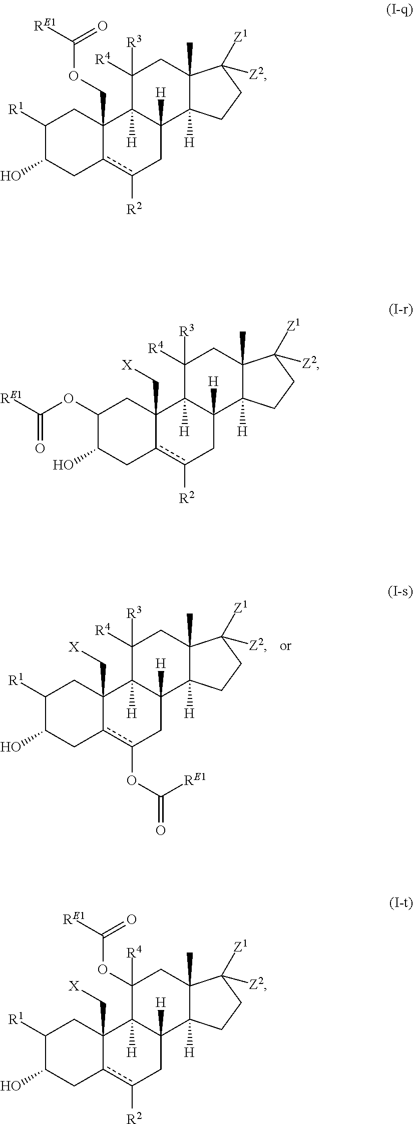
or a pharmaceutically acceptable salt, solvate, stereoisomer, tautomer, and/or isotopic variant thereof.

23. The compound of claim 22, wherein Z¹ and Z² are joined to form an alkenyl group ═CH(CN), wherein CN is in the Z configuration;

24. The compound of claim 22, wherein R¹, R², R³, and R⁴ are each H.

25. The compound of claim 22, wherein R^{E1} is —CH₃, —CH₂CN, or phenyl.

26. The compound of claim 25, wherein R^{E1} is —CH₃.

27. A pharmaceutical composition comprising a compound of claim 1 and a pharmaceutically acceptable excipient.

28. A method of inducing sedation and/or anesthesia in a subject, comprising administering to the subject an effective amount of a compound of claim 1.

29. The method of claim 28, wherein the compound is administered by intravenous administration.

30. The method of claim 28, wherein the compound is metabolized in vivo to a less active or inactive compound.
